SEQUENCE LISTING (115 sequences)

SEQ ID NO: 1-2 - skipped (2 entries)

SEQ ID NO: 3 - DNA, 10 bp, Artificial Sequence
  intronic splicing enhancer
gtagtgaggg

SEQ ID NO: 4 - DNA, 10 bp, Artificial Sequence
  intronic splicing enhancer
gttggtggtt

SEQ ID NO: 5 - DNA, 10 bp, Artificial Sequence
  intronic splicing enhancer
agttgtggtt

SEQ ID NO: 6 - DNA, 10 bp, Artificial Sequence
  intronic splicing enhancer
gtattgggtc

SEQ ID NO: 7 - DNA, 10 bp, Artificial Sequence
  intronic splicing enhancer
agtgtgaggg

SEQ ID NO: 8 - DNA, 10 bp, Artificial Sequence
  intronic splicing enhancer
gggtaatggg

SEQ ID NO: 9 - DNA, 10 bp, Artificial Sequence
  intronic splicing enhancer
tcattggggt

SEQ ID NO: 10 - DNA, 10 bp, Artificial Sequence
  intronic splicing enhancer
ggtgggggtc

SEQ ID NO: 11 - DNA, 10 bp, Artificial Sequence
  intronic splicing enhancer
ggttttgttg

SEQ ID NO: 12 - DNA, 10 bp, Artificial Sequence
  intronic splicing enhancer
tatactcccg

SEQ ID NO: 13 - DNA, 10 bp, Artificial Sequence
  intronic splicing enhancer
gtattcgatc

SEQ ID NO: 14 - skipped

SEQ ID NO: 15 - DNA, 10 bp, Artificial Sequence
  intronic splicing enhancer
gtagttccct

SEQ ID NO: 16 - DNA, 10 bp, Artificial Sequence
  intronic splicing enhancer
gttaatagta

SEQ ID NO: 17 - DNA, 10 bp, Artificial Sequence
  intronic splicing enhancer
tgctggttag

SEQ ID NO: 18 - DNA, 10 bp, Artificial Sequence
  intronic splicing enhancer
ataggtaacg

SEQ ID NO: 19 - DNA, 10 bp, Artificial Sequence
  intronic splicing enhancer
tctgaattgc

SEQ ID NO: 20 - DNA, 10 bp, Artificial Sequence
  intronic splicing enhancer
tctgggtttg

SEQ ID NO: 21 - DNA, 10 bp, Artificial Sequence
  intronic splicing enhancer
cattctcttt

SEQ ID NO: 22 - DNA, 10 bp, Artificial Sequence
  intronic splicing enhancer
gtattggtgt

SEQ ID NO: 23 - skipped

SEQ ID NO: 24 - DNA, 10 bp, Artificial Sequence
  intronic splicing enhancer
tttagatttg

SEQ ID NO: 25 - DNA, 10 bp, Artificial Sequence
  intronic splicing enhancer
ataagtactg

SEQ ID NO: 26 - DNA, 10 bp, Artificial Sequence
  intronic splicing enhancer
tagtctatta

SEQ ID NO: 27 - DNA, 52 bp, Artificial Sequence
  intronic sequence
cgaggaggcagctcctcaccctccctttctcttttgtcctgcgggtcctcag

SEQ ID NO: 28 - DNA, 52 bp, Artificial Sequence
  intronic sequence
cgaagggggcgggagtggcgggcaccgggctgacacgtgtccctcactgcag

SEQ ID NO: 29 - DNA, 52 bp, Artificial Sequence
  intronic sequence
tccgcccacatccacacctgccccacctctgactcccttctcttgactccag

SEQ ID NO: 30 - DNA, 52 bp, Artificial Sequence
  intronic sequence
ccacaggctggtccccccactgccccgccctcaccaccatctctgttcacag

SEQ ID NO: 31 - DNA, 52 bp, Artificial Sequence
  intronic sequence
tgggcccagctctgtcccacaccgcggtcacatggcaccacctctcttgcag

SEQ ID NO: 32 - DNA, 52 bp, Artificial Sequence
  intronic sequence
ggacaccttctctcctcccagattccagtaactcccaatcttctctctgcag

SEQ ID NO: 33 - DNA, 52 bp, Artificial Sequence
  intronic sequence
agggacaggccccagccgggtgctgacacgtccacctccatctcttcctcag

SEQ ID NO: 34 - DNA, 52 bp, Artificial Sequence
  intronic sequence
ggcccaccctctgccctgagagtgaccgctgtaccaacctctgtccctacag

SEQ ID NO: 35 - DNA, 52 bp, Artificial Sequence
  intronic sequence
tgggcccagctctgtcccacaccgcagtcacatggcgccatctctcttgcag

SEQ ID NO: 36 - DNA, 52 bp, Artificial Sequence
  intronic sequence
agataccttctctcttcccagatctgagtaactcccaatcttctctctgcag

SEQ ID NO: 37 - DNA, 52 bp, Artificial Sequence
  intronic sequence
acgcatccacctccatcccagatccccgtaactcccaatcttctctctgcag

SEQ ID NO: 38 - DNA, 52 bp, Artificial Sequence
  intronic sequence
acgcgtccacctccatcccagatccccgtaactcccaatcttctctctgcag

SEQ ID NO: 39 - DNA, 52 bp, Artificial Sequence; identical to SEQ ID NO: 37
  intronic sequence

SEQ ID NO: 40 - DNA, 52 bp, Artificial Sequence; identical to SEQ ID NO: 37
  intronic sequence

SEQ ID NO: 41 - DNA, 52 bp, Artificial Sequence
  intronic sequence
gacccaccctctgccctgggagtgaccgctgtgccaacctctgtccctacag

SEQ ID NO: 42 - DNA, 52 bp, Artificial Sequence; identical to SEQ ID NO: 31
  intronic sequence

SEQ ID NO: 43 - DNA, 52 bp, Artificial Sequence
  intronic sequence
agacaccttctctcctcccagatctgagtaactcccaatcttctctctgcag

SEQ ID NO: 44 - DNA, 52 bp, Artificial Sequence
  intronic sequence
agggacaggccccagccgggtgctgacgcatccacctccatctcttcctcag

SEQ ID NO: 45 - DNA, 52 bp, Artificial Sequence
  intronic sequence
ggcccaccctctgccctgggagtgaccgctgtgccaacctctgtccctacag

SEQ ID NO: 46 - DNA, 52 bp, Artificial Sequence
  intronic sequence
gtgagtctgctgtctggggatagcggggagccaggtgtactgggccaggcaa

SEQ ID NO: 47 - DNA, 52 bp, Artificial Sequence
  intronic sequence
gtgagtcccactgcagccccctcccagtcttctctgtccaggcaccaggcca

SEQ ID NO: 48 - DNA, 52 bp, Artificial Sequence
  intronic sequence
gtaagatggctttccttctgcctcctttctctgggcccagcgtcctctgtcc

SEQ ID NO: 49 - DNA, 52 bp, Artificial Sequence
  intronic sequence
gtgagtcctcacaacctctctcctgctttaactctgaagggttttgctgcat

SEQ ID NO: 50 - DNA, 52 bp, Artificial Sequence
  intronic sequence
gtgagtcctcaccaccccctctctgagtccacttagggagactcagcttgcc

SEQ ID NO: 51 - DNA, 52 bp, Artificial Sequence
  intronic sequence
gtaagaatggccactctagggcctttgttttctgctactgcctgtggggttt

SEQ ID NO: 52 - DNA, 52 bp, Artificial Sequence
  intronic sequence
catggtgacttcctacagtggacgctgagatcctgctctgcttccctcctag

SEQ ID NO: 53 - DNA, 52 bp, Artificial Sequence
  intronic sequence
gtgaggacgtcacctgggccctgccccagtctcagctcgaccctcgagcttg

SEQ ID NO: 54 - protein, 8 aa, Artificial Sequence
  cleavage tag
LEVLFQGP

SEQ ID NO: 55 - protein, 5 aa, Artificial Sequence
  cleavage tag
DDDDK

SEQ ID NO: 56 - protein, 4 aa, Artificial Sequence
  cleavage tag
IEGR

SEQ ID NO: 57 - protein, 7 aa, Artificial Sequence
  cleavage tag
ENLYFQG

SEQ ID NO: 58 - protein, 6 aa, Artificial Sequence
  cleavage tag
LVPRGS

SEQ ID NO: 59 - protein, 8 aa, Artificial Sequence
  self-processing site
  Xaa (2)..(2): wherein Xaa is Val or Ile
  Xaa (4)..(4): wherein Xaa may be any (naturally occurring) amino acid
DXEXNPGP

SEQ ID NO: 60 - protein, 18 aa, Artificial Sequence
  self-processing site
EGRGSLLTCGDVEENPGP

SEQ ID NO: 61 - protein, 22 aa, Artificial Sequence
  self-processing site
VKQTLNFDLLKLAGDVESNPGP

SEQ ID NO: 62 - protein, 19 aa, Artificial Sequence
  self-processing site
ATNFSLLKQAGDVEENPGP

SEQ ID NO: 63 - protein, 22 aa, Artificial Sequence
  self-processing site
GSGATNFSLLKQAGDVEENPGP

SEQ ID NO: 64 - protein, 26 aa, Artificial Sequence
  self-processing site
RKRRGSGATNFSLLKQAGDVEENPGP

SEQ ID NO: 65 - protein, 30 aa, Artificial Sequence
  twin StrepTag
SAWSHPQFEKGGGSGGGSGGSAWSHPQFEK

SEQ ID NO: 66 - protein, 15 aa, Artificial Sequence
  AviTag
GLNDIFEAQKIEWHE

SEQ ID NO: 67 - protein, 26 aa, Artificial Sequence
  Calmodulin-tag
KRRWKKNFIAVSAANRFKKISSSGAL

SEQ ID NO: 68 - protein, 6 aa, Artificial Sequence
  polyglutamate tag
EEEEEE

SEQ ID NO: 69 - protein, 13 aa, Artificial Sequence
  E-tag
GAPVPYPDPLEPR

SEQ ID NO: 70 - protein, 8 aa, Artificial Sequence
  FLAG-tag
DYKDDDDK

SEQ ID NO: 71 - protein, 9 aa, Artificial Sequence
  HA-tag
YPYDVPDYA

SEQ ID NO: 72 - protein, 6 aa, Artificial Sequence
  His-tag
HHHHHH

SEQ ID NO: 73 - protein, 10 aa, Artificial Sequence
  Myc-tag
EQKLISEEDL

SEQ ID NO: 74 - protein, 18 aa, Artificial Sequence
  NE-tag
TKENPRSNQEESYDDNES

SEQ ID NO: 75 - protein, 15 aa, Artificial Sequence
  S-tag
KETAAAKFERQHMDS

SEQ ID NO: 76 - protein, 38 aa, Artificial Sequence
  SBP-tag
MDEKTTGWRGGHVVEGLAGELEQLRARLEHHPQGQREP

SEQ ID NO: 77 - protein, 13 aa, Artificial Sequence
  Softag 1
SLAELLNAGLGGS

SEQ ID NO: 78 - protein, 8 aa, Artificial Sequence
  Softag 3
TQDPSRVG

SEQ ID NO: 79 - protein, 8 aa, Artificial Sequence
  Strep-tag
WSHPQFEK

SEQ ID NO: 80 - protein, 6 aa, Artificial Sequence
  TC tag
CCPGCC

SEQ ID NO: 81 - protein, 14 aa, Artificial Sequence
  V5 tag
GKPIPNPLLGLDST

SEQ ID NO: 82 - protein, 11 aa, Artificial Sequence
  VSV-tag
YTDIEMNRLGK

SEQ ID NO: 83 - protein, 8 aa, Artificial Sequence
  Xpress tag
DLYDDDDK

SEQ ID NO: 84 - protein, 16 aa, Artificial Sequence
  Isopeptag
TDKDMTITFTNKKDAE

SEQ ID NO: 85 - protein, 13 aa, Artificial Sequence
  SpyTag
AHIVMVDAYKPTK

SEQ ID NO: 86 - protein, 12 aa, Artificial Sequence
  SnoopTag
KLGDIEFIKVNK

SEQ ID NO: 87 - protein, 10 aa, Artificial Sequence
  Ty1 tag
EVHTNQDPLD

SEQ ID NO: 88 - protein, 98 aa, Artificial Sequence
  mutated LAIR1 fragment
EDLPRPSISAEPGTVIPLGSHVTFVCRGPVGVQTFRLERERNYLYSDTEDVSQTSPSESEARFRIDSVNAGNAGLFRCIYYKSRKWSEQSDYLELVVK

SEQ ID NO: 89 - protein, 120 aa, Artificial Sequence
  PD-1 fragment
DSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVT

SEQ ID NO: 90 - protein, 95 aa, Artificial Sequence
  SLAM fragment
EQVSTPEIKVLNKTQENGTCTLILGCTVEKGDHVAYSWSEKAGTHPLNPANSSHLLSLTLGPQHADNIYICTVSNPISNNSQTFSPWPGCRTDPS

SEQ ID NO: 91 - protein, 117 aa, Artificial Sequence
  T3-VHH
MAQVQLVESGGGLVQAGGSLTLSCAASGSTSRSYALGWFRQAPGKEREFVAHVGQTAEFAQGRFTISRDFAKNTVSLQMNDLKSDDTAIYYCVASNRGWSPSRVSYWGQGTQVTVSS

SEQ ID NO: 92 - protein, 248 aa, Artificial Sequence
  TT39.7-scFv
QITLKESGPTLVKPTQTLTLTCTFSGFSLSTSRVGVGWIRQPPGKALEWLSLIYWDDEKHYSPSLKNRVTISKDSSKNQVVLTLTDMDPVDTGTYYCAHRGVDTSGWGFDYWGQGALVTVSSGGGGSGGGGSGGGGSQSALTQPASVSGSPGQSITISCSGAGSDVGGHNFVSWYQQYPGKAPKLMIYDVKNRPSGVSYRFSGSKSGYTASLTISGLQAEDEATYFCSSYSSSSTLIIFGGGTRLTVL

SEQ ID NO: 93 - protein, 125 aa, Artificial Sequence
  F4-VHH
QVQLQESGGGLVQPGGSLRLSCAASGFTLDYYYIGWFRQAPGKEREAVSCISGSSGSTYYPDSVKGRFTISRDNAKNTVYLQMNSLKPEDTAVYYCATIRSSSWGGCVHYGMDYWGKGTQVTVSS

SEQ ID NO: 94 - protein, 249 aa, Artificial Sequence
  MPE8-scFv
EVQLVESGGGLVKPGGSLRLSCAASGFTFSSYSMNWVRQAPGKGLEWVSSISASSSYSDYADSAKGRFTISRDNAKTSLFLQMNSLRAEDTAIYFCARARATGYSSITPYFDIWGQGTLVTVSSGGGGSGGGGSGGGGSQSVVTQPPSVSGAPGQRVTISCTGSSSNIGAGYDVHWYQQLPGTAPKLLIYDNNNRPSGVPDRFSASKSGTSASLAITGLQAEDEADYYCQSYDRNLSGVFGTGTKVTVL

SEQ ID NO: 95 - DNA, 144 bp, Artificial Sequence
  SV40 nuclear localization signal
tggttgctgactaattgagatgcatgctttgcatacttctgcctgctggggagcctggggactttccacacctggttgctgactaattgagatgcatgctttgcatacttctgcctgctggggagcctggggactttccacacc

SEQ ID NO: 96 - DNA, 27 bp, Artificial Sequence
  primer
cacccttgaaagtagcccatgccttcc

SEQ ID NO: 97 - DNA, 30 bp, Artificial Sequence
  primer
cctgcctcccagtgtcctgcattacttctg

SEQ ID NO: 98 - DNA, 26 bp, Artificial Sequence
  primer
ggaacgcagtgtagactcagctgagg

SEQ ID NO: 99 - DNA, 590 bp, Artificial Sequence
  DNA substrate
ttgtgagcaagtctcagggtcctcactgtcaactgggaaaaaactctgcagtgatgagaatcacatgcacgtagaaggtgcaggaggcgtgggaatgttctaaggttgggctgtggtcatggctgcataactctataaaattgctaaaatccctgaattgtgatgctaaaatgacgtgtgtggcatggtgacttcctacagtggacgctgagatcctgctctgcttccctcctagaagatctgcccagaccctccatctcggctgagccaggcaccgtgatccccctggggagccatgtgactttcgtgtgccggggcccggttggggttcaaacattccgcctggagagggacagtagatccacatacaatgatactgaagatgtgtctcaagctagtccatctgagtcagaggccagattccgcattgactcagtaagagaaggaaatgccgggctttatcgctgcatctattataagccccctaaatggtctgagcagagtgactacctggagctgctggtgaaaggtgaggacgtcacctgggccctgccccagtctcagctcgaccctcgagcttgtccccaggt

SEQ ID NO: 100 - DNA, 23 bp, Artificial Sequence
  primer
ccacctccaaacggcaggcatcc

SEQ ID NO: 101 - DNA, 24 bp, Artificial Sequence
  primer
ccaaaggccgcatgaccatcacgc

SEQ ID NO: 102 - DNA, 26 bp, Artificial Sequence
  primer
cctcagctgagtctacactgcgttcc

SEQ ID NO: 103 - DNA, 30 bp, Artificial Sequence
  primer
ctgaggacccgcaggacaaaagagaaaggg

SEQ ID NO: 104 - DNA, 29 bp, Artificial Sequence
  primer
ggtcaccgtctcctcaggtaagaatggcc

SEQ ID NO: 105 - DNA, 26 bp, Artificial Sequence
  primer
gccttttcagtttcggtcagcctcgc

SEQ ID NO: 106 - DNA, 29 bp, Artificial Sequence
  primer
gcgggtcctcagaagatctgcccagaccc

SEQ ID NO: 107 - DNA, 32 bp, Artificial Sequence
  primer
ggccattcttacctttcaccagcagctccagg

SEQ ID NO: 108 - DNA, 32 bp, Artificial Sequence
  primer
gcgggtcctcaggggaagatctgcccagaccc

SEQ ID NO: 109 - DNA, 44 bp, Artificial Sequence
  primer
ggccattcttacctgaggagacggctttcaccagcagctccagg

SEQ ID NO: 110 - DNA, 1965 bp, Artificial Sequence
  DNA substrate
cctcagctgagtctacactgcgttccccatcacactcaccctccctatactcactcccaggcctgggttgtctgcctggggagacttcagggtagctggagtgtgactgagctgggggcagcagaagctgggctggagggactctattggctgcctgcggggtgtgtggctccaggcttcacattcaggtatgcaacctgggccctccagctgcatgtgctgggagctgagtgtgtgcagcacctacgtgctgatgcctcgggggaaagcaggcctggtccacccaaacctgagccctcagccattctgagcagggagccaggggcagtcaggcctcagagtgcagcagggcagccagctgaatggtggcagggatggctcagcctgctccaggagaccccaggtctgtccaggtgttcagtgctgggccctgcagcaggatgggctgaggcctgcagccccagcagccttggacaaagacctgaggcctcaccacggccccgccacccctgatagccatgacagtctgggctttggaggcctgcaggtgggctcggccttggtggggcagccacagcgggacgcaagtagtgagggcactcagaacgccactcagccccgacaggcagggcacgaggaggcagctcctcaccctccctttctcttttgtcctgcgggtcctcagaagatctgcccagaccctccatctcggctgagccaggcaccgtgatccccctggggagccatgtgactttcgtgtgccggggcccggttggggttcaaacattccgcctggagagggacagtagatccacatacaatgatactgaagatgtgtctcaagctagtccatctgagtcagaggccagattccgcattgactcagtaagagaaggaaatgccgggctttatcgctgcatctattataagccccctaaatggtctgagcagagtgactacctggagctgctggtgaaaggtaagaatggccactctagggcctttgttttctgctactgcctgtggggtttcctgagcattgcaggttggtcctcggggcatgttccgaggggacctgggcggactggccaggaggggatgggcactggggtgccttgaggatctgggagcctctgtggattttccgatgcctttggaaaatgggactcaggttgggtgcgtctgatggagtaactgagcctgggggcttggggagccacatttggacgagatgcctgaacaaaccaggggtcttagtgatggctgaggaatgtgtctcaggagcggtgtctgtaggactgcaagatcgctgcacagcagcgaatcgtgaaatattttctttagaattatgaggtgcgctgtgtgtcaacctgcatcttaaattctttattggctggaaagagaactgtcggagtgggtgaatccagccaggagggacgcgtagccccggtcttgatgagagcagggttgggggcaggggtagcccagaaacggtggctgccgtcctgacaggggcttagggaggctccaggacctcagtgccttgaagctggtttccatgagaaaaggattgtttatcttaggaggcatgcttactgttaaaagacaggatatgtttgaagtggcttctgagaaaaatggttaagaaaattatgacttaaaaatgtgagagattttcaagtatattaatttttttaactgtccaagtatttgaaattcttatcatttgattaacacccatgagtgatatgtgtctggaattgaggccaaagcaagctcagctaagaaatactagcacagtgctgtcggccccgatgcgggactgcgttttgaccatcataaatcaagtttatttttttaattaattgagcgaagctggaagcagatgatgaattagagtcaagatggctgcatgggggtctccggcacccacagcaggtggcaggaagcaggtcaccgcgagag

SEQ ID NO: 111 - DNA, 294 bp, Artificial Sequence
  Nucleotide sequence encoding (poly)peptide of interest
aagatctgcccagaccctccatctcggctgagccaggcaccgtgatccccctggggagccatgtgactttcgtgtgccggggcccggttggggttcaaacattccgcctggagagggacagtagatccacatacaatgatactgaagatgtgtctcaagctagtccatctgagtcagaggccagattccgcattgactcagtaagagaaggaaatgccgggctttatcgctgcatctattataagccccctaaatggtctgagcagagtgactacctggagctgctggtgaaag

SEQ ID NO: 112 - DNA, 235 bp, Artificial Sequence
  intronic sequence
ttgtgagcaagtctcagggtcctcactgtcaactgggaaaaaactctgcagtgatgagaatcacatgcacgtagaaggtgcaggaggcgtgggaatgttctaaggttgggctgtggtcatggctgcataactctataaaattgctaaaatccctgaattgtgatgctaaaatgacgtgtgtggcatggtgacttcctacagtggacgctgagatcctgctctgcttccctcctag

SEQ ID NO: 113 - DNA, 61 bp, Artificial Sequence
  intronic sequence
gtgaggacgtcacctgggccctgccccagtctcagctcgaccctcgagcttgtccccaggt

SEQ ID NO: 114 - DNA, 685 bp, Artificial Sequence
  intronic sequence
cctcagctgagtctacactgcgttccccatcacactcaccctccctatactcactcccaggcctgggttgtctgcctggggagacttcagggtagctggagtgtgactgagctgggggcagcagaagctgggctggagggactctattggctgcctgcggggtgtgtggctccaggcttcacattcaggtatgcaacctgggccctccagctgcatgtgctgggagctgagtgtgtgcagcacctacgtgctgatgcctcgggggaaagcaggcctggtccacccaaacctgagccctcagccattctgagcagggagccaggggcagtcaggcctcagagtgcagcagggcagccagctgaatggtggcagggatggctcagcctgctccaggagaccccaggtctgtccaggtgttcagtgctgggccctgcagcaggatgggctgaggcctgcagccccagcagccttggacaaagacctgaggcctcaccacggccccgccacccctgatagccatgacagtctgggctttggaggcctgcaggtgggctcggccttggtggggcagccacagcgggacgcaagtagtgagggcactcagaacgccactcagccccgacaggcagggcacgaggaggcagctcctcaccctccctttctcttttgtcctgcgggtcctcag

SEQ ID NO: 115 - DNA, 986 bp, Artificial Sequence
  intronic sequence
gtaagaatggccactctagggcctttgttttctgctactgcctgtggggtttcctgagcattgcaggttggtcctcggggcatgttccgaggggacctgggcggactggccaggaggggatgggcactggggtgccttgaggatctgggagcctctgtggattttccgatgcctttggaaaatgggactcaggttgggtgcgtctgatggagtaactgagcctgggggcttggggagccacatttggacgagatgcctgaacaaaccaggggtcttagtgatggctgaggaatgtgtctcaggagcggtgtctgtaggactgcaagatcgctgcacagcagcgaatcgtgaaatattttctttagaattatgaggtgcgctgtgtgtcaacctgcatcttaaattctttattggctggaaagagaactgtcggagtgggtgaatccagccaggagggacgcgtagccccggtcttgatgagagcagggttgggggcaggggtagcccagaaacggtggctgccgtcctgacaggggcttagggaggctccaggacctcagtgccttgaagctggtttccatgagaaaaggattgtttatcttaggaggcatgcttactgttaaaagacaggatatgtttgaagtggcttctgagaaaaatggttaagaaaattatgacttaaaaatgtgagagattttcaagtatattaatttttttaactgtccaagtatttgaaattcttatcatttgattaacacccatgagtgatatgtgtctggaattgaggccaaagcaagctcagctaagaaatactagcacagtgctgtcggccccgatgcgggactgcgttttgaccatcataaatcaagtttatttttttaattaattgagcgaagctggaagcagatgatgaattagagtcaagatggctgcatgggggtctccggcacccacagcaggtggcaggaagcaggtcaccgcgagag

(19) United States
(12) Patent Application Publication
(10) Pub. No.: US 2021/0254106 A1
(43) Pub. Date: Aug. 19, 2021

(54) ENGINEERING B LYMPHOCYTES BY UTILIZING ENDOGENOUS ACTIVATION-INDUCED CYTIDINE DEAMINASE

(71) Applicant: INSTITUTE FOR RESEARCH IN BIOMEDICINE, Bellinzona (CH)

(72) Inventors: Antonio LANZAVECCHIA, Porza (CH); Kathrin DE LA ROSA, Beverungen (DE); Philipp PAPARODITIS, Nicosia (CY)

(21) Appl. No.: 17/059,112

(22) PCT Filed: May 29, 2019
(86) PCT No.: PCT/EP2019/064109

§ 371 Date: Nov. 25, 2020

(30) Foreign Application Priority Data
May 30, 2018 (EP) PCT/EP2018/064299

(51) Int. Cl. C12N 15/90; C12N 9/78; C12N 5/0781; A61K 35/17; C12P 21/02; C07K 16/00; A61P 37/02

(52) U.S. Cl. CPC C12N 15/907; C12N 9/78; C12Y 305/04005; C12N 5/0635; A61K 35/17; C07K 2317/14; C07K 16/00; A61P 37/02; C12N 2510/02; C12N 2501/2304; C12N 2501/52; C12P 21/02

(57) ABSTRACT

The present invention provides a method for engineering B lymphocytes by utilizing activation-induced cytidine deaminase of the B lymphocyte. Thereby, use of engineered nucleases, such as Cas nuclease, can be avoided. Engineered B cells are useful to produce customized antibodies and for B cell therapy. Accordingly, the present invention also provides engineered B cells and customized antibodies produced by engineered B cells.

The present invention relates to the field of engineered B lymphocytes, in particular for production of antibodies. In particular the present invention relates to editing of immunoglobulin genes in B cells, such that the engineered B cells are able to produce customized antibodies. Accordingly, the present invention provides a method for engineering B cells and B cells engineered according to the method of the present invention. Such engineered B cells and the customized antibodies produced by the B cells are useful in a variety of medical applications, including prevention and therapy of diseases targeted by the engineered antibody as well as diagnostic approaches, e.g. for detection of an antigen in a (isolated) sample.

The use of therapeutic monoclonal antibodies has emerged as ground-breaking approach to specifically target a wide variety of diseases including immune disorders, cancers, and infections. Currently, 65 monoclonal antibodies (mAbs) are approved by the FDA for clinical use and more than 350 mAbs are in clinical trials, which demonstrates the power of this therapeutic approach and of recombinant antibody engineering.

The potential of therapeutic antibodies as powerful tools for treatment of numerous diseases emerged in particular since 1975, when Köhler and Milstein developed a procedure for producing mAbs (Köhler G, Milstein C: Continuous cultures of fused cells secreting antibody of predefined specificity. Nature. 1975 Aug. 7; 256(5517):495-7). The first mAbs were produced in mice and, when administered to patients, those murine antibodies faced serious problems as they were recognized as foreign molecules. This resulted in elimination by the human immune system and in allergic responses ranging from a mild rash to renal failure. Moreover, these murine antibodies were not able to interact properly with components of the human immune system and their biological efficacy was severely restricted (for review see Chames P, Van Regenmortel M, Weiss E, Baty D. Therapeutic antibodies: successes, limitations and hopes for the future. British Journal of Pharmacology. 2009; 157(2):220-233. doi:10.1111/j.1476-5381.2009.00190.x.).

To avoid those problems, strategies were developed to make the murine antibodies more “human”. One approach was the development of chimeric antibodies, in which murine variable domains were fused to human constant domains, resulting in an antibody, which is approx. 70% human and which has a fully human Fc portion (Neuberger M S, Williams G T, Mitchell E B, Jouhal S S, Flanagan J G, Rabbitts T H: A hapten-specific chimaeric IgE antibody with human physiological effector function. Nature. 1985 Mar. 21-27; 314(6008):268-70). To further decrease murine part of mAbs, “humanized” antibodies were developed, in which the hypervariable loops of a fully human antibody were replaced with the hypervariable loops of the murine antibody of interest by “complementarity-determining region (CDR) grafting”. Humanized antibodies contain 85-90% human sequences and are even less immunogenic than chimeric antibodies. Most of the approved mAbs are chimeric or humanized (for review see Chames P, Van Regenmortel M, Weiss E, Baty D. Therapeutic antibodies: successes, limitations and hopes for the future. British Journal of Pharmacology. 2009; 157(2):220-233. doi:10.1111/j.476-5381.2009.00190.x.). However, humanizing is technically demanding and can result in a loss of antibody activity (i.e., in a loss of function).

Another approach for developing therapeutic antibodies relates to in vitro display technologies, such as phage display (McCafferty), Griffiths A D, Winter G, Chiswell D J: Phage antibodies: filamentous phage displaying antibody variable domains. Nature. 1990 Dec. 6; 348(6301):552-4). Thereby, human antibodies or antibody fragments are displayed on the surface of a simple organism, such as phage, bacteria or yeast for screening. However, such library systems do not contain full-length antibodies and the antibodies are expressed by bacteria or yeast rather than by human cells. Such expression systems cannot reflect human post-translational modifications. In particular, antibodies produced in vitro do often not resemble natural, human antibody glycosylation patterns, which are however crucial for antibody effectiveness as they influence effector functions and downstream activation of the immune system.

Furthermore, transgenic “humanized” mice may be used to produce antibodies from human genes (Lonberg N. Human monoclonal antibodies from transgenic mice. In: Chernajovsky Y, Nissim A, editors. Therapeutic Antibodies. Handbook of Experimental Pharmacology, Volume 181. Berlin Heidelberg: Springer-Verlag; 2008. pp. 69-97. Eds.). However, as this technology relies on immunization of mice with an antigen, it is limited to the production of antibodies for antigens, which can be recognized by the immune system of a mouse.

Accordingly, the “gold standard” for producing therapeutic antibodies is the use of isolated human B lymphocytes, which utilizes the “natural” way of human antibody production (Traggiai E, Becker S, Subbarao K, Kolesnikova L, Uematsu Y, Gismondo M R, Murphy B R, Rappuoli R, Lanzavecchia A. An efficient method to make human monoclonal antibodies from memory B cells: potent neutralization of SARS coronavirus. Nat Med. 2004 August; 10(8):871-5. Epub 2004 Jul. 11; Lanzavecchia A, Bernasconi N, Traggiai E, Ruprecht C R, Corti D, Sallusto F. Understanding and making use of human memory B cells. Immunol Rev. 2006 June; 211:303-9). Accordingly, B cells were traditionally used to obtain natural human antibodies and human antibody libraries based on the natural B cell genome (Duvall M R, Fiorini R N. Different approaches for obtaining antibodies from human B cells. Curr Drug Discov Technol. 2014 March; 11(1):41-7). Only recently, with the development of genome editing tools like CRISPR/Cas9, Zinc finger nuclease and TALENs (Transcription Activator-Like Effector Nucleases), human B cells also emerged as target for immunoglobulin gene editing, such that also customized recombinant antibodies could be produced by isolated human B cells.

For example, Cheong and colleagues reported the application of the CRISPR/Cas9 technology to edit immunoglobulin genes by delivering Cas9 and guide-RNA with retro- or lentivirus to B cells, thereby inducing immunoglobulin class-switch recombination (Cheong T C, Compagno M, Chiarle R. Editing of mouse and human immunoglobulin genes by CRISPR-Cas9 system. Nat Commun. 2016 Mar. 9; 7:10934. doi: 10.1038/ncomms10934).

In addition, WO 2016/161446 also describes the use of the CRISPR/Cas9 technology to engineer human B cells. In addition, WO 2016/161446 also suggests the use of other engineered nucleases, such as zinc finger nucleases and TALENS for genetically modifying B cells.

In general, genome editing approaches using engineered nucleases, such as CRISPR/Cas9, Zinc finger nuclease and TALENs, recently emerged as powerful tool in biotechnology with promising therapeutic potential. However, due to the working mechanism of the engineered nucleases and their delivery requirements, also major safety concerns arise for the use of genome editing by engineered nucleases in clinical applications.

Thereby, the major concern relates to undesired off-target cleavage and mutations. Unintended interactions of engineered nucleases and consequent cleavage of non-target sites were reported despite specific targeting by the engineered nucleases (Zhang X H, Tee L Y, Wang X G, Huang Q S, Yang S H. Off-target Effects in CRISPR/Cas9-mediated Genome Engineering. Mol Ther Nucleic Acids. 2015 Nov. 17; 4:e264. doi: 10.1038/mtna.2015.37; Shim G, Kim D, Park G T, Jin H, Suh S K, Oh Y K. Therapeutic gene editing: delivery and regulatory perspectives. Acta Pharmacol Sin. 2017 June; 38(6):738-753. doi: 10.1038/aps.2017.2). For example, in the CRISPR/Cas9 system, sgRNA can bind to a mismatched sequence with partial homology.

In addition, tumorigenicity of exogenous gene editing tools represent another important safety issue, whereby in particular, off-target mutations (for example near proto-oncogenes) may result in the development of cancer. In other words, the generation of off-target mutations bears the risk of functional abnormalities and initiation of cancer.

Another important safety concern relates to the immunogenicity of the engineered nucleases, since CRISPR/Cas9, Zinc finger nuclease and TALENs are all exogenous and foreign to the human body. Accordingly, engineered nucleases can elicit an immune response (Dai W J, Zhu L Y, Yan Z Y, Xu Y, Wang Q L, Lu X J. CRISPR-Cas9 for in vivo Gene Therapy: Promise and Hurdles. Mol Ther Nucleic Acids. 2016; 5:e349. doi: 10.1038/mtna.2016.58). Moreover, also viral vectors used for the delivery of engineered nucleases may be immunogenic and result in production of antibodies and T-cell immune responses limiting the repeated use of the same viral vectors (Zaiss A K, Muruve D A. Immune responses to adeno-associated virus vectors. Curr Gene Ther. 2005 June; 5(3):323-31). Moreover, viral vectors bear the risk of chromosomal integration and germline transmission.

Accordingly, there is a need for the development of safer B cell genome editing tools.

In view thereof, it is the object of the present invention to provide a novel method for engineering B cells, which overcomes the drawbacks of the prior art described above. In particular, it is an object of the present invention to provide a safer method for engineering B cells. For example, it is an object of the present invention to provide a method for engineering B cells, which lowers the risk for undesired off-target mutations. These objects are achieved by means of the subject-matter set out below and in the appended claims.

Although the present invention is described in detail below, it is to be understood that this invention is not limited to the particular methodologies, protocols and reagents described herein as these may vary. It is also to be understood that the terminology used herein is not intended to limit the scope of the present invention which will be limited only by the appended claims. Unless defined otherwise, all technical and scientific terms used herein have the same meanings as commonly understood by one of ordinary skill in the art.

In the following, the elements of the present invention will be described. These elements are listed with specific embodiments, however, it should be understood that they may be combined in any manner and in any number to create additional embodiments. The variously described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

Throughout this specification and the claims which follow, unless the context requires otherwise, the term “comprise”, and variations such as “comprises” and “comprising”, will be understood to imply the inclusion of a stated member, integer or step but not the exclusion of any other non-stated member, integer or step. The term “consist of” is a particular embodiment of the term “comprise”, wherein any other non-stated member, integer or step is excluded. In the context of the present invention, the term “comprise” encompasses the term “consist of”. The term “comprising” thus encompasses “including” as well as “consisting” e.g., a composition “comprising” X may consist exclusively of X or may include something additional e.g., X+Y.

The terms “a” and “an” and “the” and similar reference used in the context of describing the invention (especially in the context of the claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. No language in the specification should be construed as indicating any non-claimed element essential to the practice of the invention.

The word “substantially” does not exclude “completely” e.g., a composition which is “substantially free” from Y may be completely free from Y. Where necessary, the word “substantially” may be omitted from the definition of the invention.

The term “about” in relation to a numerical value x means x±10%.

As used herein, the terms “peptide”, “polypeptide”, “protein” refer to peptides, oligopeptides, or proteins including fusion proteins, respectively, comprising at least two amino acids joined to each other, preferably by a normal peptide bond, or, alternatively, by a modified peptide bond, such as for example in the cases of isosteric peptides. The term “(poly)peptide” refers to a peptide and/or a polypeptide. In particular, the terms “peptide”, “polypeptide”, “protein” also include “peptidomimetics” which are defined as peptide analogs containing non-peptidic structural elements, which peptides are capable of mimicking or antagonizing the biological action(s) of a natural parent peptide. A peptidomimetic lacks classical peptide characteristics such as enzymatically scissile peptide bonds. A peptide, polypeptide or protein may be composed of any of the 20 amino acids defined by the genetic code. Moreover, a peptide, polypeptide or protein may also comprise amino acids other than the 20 amino acids defined by the genetic code in addition to these amino acids, or it can be composed of amino acids other than the 20 amino acids defined by the genetic code. In particular, a peptide, polypeptide or protein in the context of the present invention can equally be composed of amino acids modified by natural processes, such as post-translational maturation processes or by chemical processes, which are well known to a person skilled in the art. Such modifications are fully detailed in the literature. These modifications can appear anywhere in the polypeptide: in the peptide skeleton, in the amino acid chain or even at the carboxy- or amino-terminal ends. In particular, a peptide or polypeptide can be branched following an ubiquitination or be cyclic with or without branching. This type of modification can be the result of natural or synthetic post-translational processes that are well known to a person skilled in the art. The terms “peptide”, “polypeptide”, “protein” in the context of the present invention in particular also include modified peptides, polypeptides and proteins. For example, peptide, polypeptide or protein modifications can include acetylation, acylation, ADP-ribosylation, amidation, covalent fixation of a nucleotide or of a nucleotide derivative, covalent fixation of a lipid or of a lipidic derivative, the covalent fixation of a phosphatidylinositol, covalent or non-covalent cross-linking, cyclization, disulfide bond formation, demethylation, glycosylation including pegylation, hydroxylation, iodization, methylation, myristoylation, oxidation, proteolytic processes, phosphorylation, prenylation, racemization, seneloylation, sulfatation, amino acid addition such as arginylation or ubiquitination. Such modifications are fully detailed in the literature (Proteins Structure and Molecular Properties (1993) 2nd Ed., T. E. Creighton, New York; Post-translational Covalent Modifications of Proteins (1983) B. C. Johnson, Ed., Academic Press, New York; Seifter et al. (1990) Analysis for protein modifications and nonprotein cofactors, Meth. Enzymol. 182: 626-646 and Rattan et al., (1992) Protein Synthesis: Post-translational Modifications and Aging, Ann NY Acad Sci, 663: 48-62). Accordingly, the terms “peptide”, “polypeptide”, “protein” preferably include for example lipopeptides, lipoproteins, glycopeptides, glycoproteins and the like.

Preferably, however, a protein, polypeptide or peptide is a “classical” peptide, polypeptide or protein, whereby a “classical” peptide, polypeptide or protein is typically composed of amino acids selected from the 20 amino acids defined by the genetic code, linked to each other by a normal peptide bond.

The term “heavy chain” (of an antibody or antibody fragment) as used herein refers to a polypeptide which is to be associated with another polypeptide (the “light chain”). In particular, the heavy chain and the light chain are associated through a disulfide bond. The heavy chain may comprise one, two, three or four antibody heavy constant domains. In a preferred embodiment, it comprises three antibody heavy constant domains: CH1, CH2 and CH3, and a hinge region between CH1 and CH2. Said heavy chain constant domains may be derived from an antibody which is murine, chimeric, synthetic, humanized or human, and monoclonal or polyclonal. The heavy chain may comprise one or more variable domains, preferably variable domains of an antibody heavy chain (VH).

The term “light chain” (of an antibody or antibody fragment) as used herein refers to a polypeptide which is to be associated with another polypeptide (the “heavy chain”). In particular, the heavy chain and the light chain are associated through a disulfide bond. The light chain may comprise an antibody light chain constant region CL. Said light chain constant region may be derived from an antibody which is murine, chimeric, synthetic, humanized or human, and monoclonal or polyclonal. The second polypeptide chain may comprise one or more variable domains, preferably variable domains of an antibody light chain (VL).

In general, an “antibody” is a protein that binds specifically to an antigen. Typically, an antibody comprises a unique structure that enables it to bind specifically to its corresponding antigen, but—in general—antibodies have a similar structure and are, in particular, also known as immunoglobulins (Ig). As used herein, the term “antibody” encompasses various forms of antibodies including, without being limited to, whole antibodies, antibody fragments, in particular antigen binding fragments, human antibodies, chimeric antibodies, humanized antibodies, recombinant antibodies and genetically engineered antibodies (variant or mutant antibodies) as long as the characteristic properties according to the invention are retained. Although the specification, including the claims, may, in some places, refer explicitly to antigen binding fragment(s), antibody fragment(s), variant(s) and/or derivative(s) of antibodies, it is understood that the term “antibody” includes all categories of antibodies, namely, antigen binding fragment(s), antibody fragment(s), variant(s) and derivative(s) of antibodies.

As used herein, the terms “antigen binding fragment,” “fragment,” and “antibody fragment” are used interchangeably and refer to any fragment of an antibody. In particular, the terms “antigen binding fragment,” “fragment,” and “antibody fragment” refer herein to any fragment of an antibody that retains (i) the antigen-binding activity of the antibody and/or (ii) an additional functionality provided by a (additional) functional domain of the antibody as described herein, for example a binding activity provided by an (independent) binding site. In antibody fragments according to the present invention the characteristic properties according to the invention are retained. In general, examples of antibody fragments include, but are not limited to, a single chain antibody, Fab, Fab′ or F(ab′)₂. Fragments of the antibodies can be obtained from antibodies by methods that include digestion with enzymes, such as pepsin or papain, and/or by cleavage of disulfide bonds by chemical reduction. Alternatively, fragments of the antibodies can be obtained by cloning and expression of part of the sequences of the heavy or light chains. Further, the term “antibody” as used herein includes both antibodies and antibody fragments.

The term “human antibody”, as used herein, is intended to include antibodies having variable and constant regions derived from human immunoglobulin sequences. Human antibodies are well-known in the state of the art (van Dijk, M. A., and van de Winkel, J. G., Curr. Opin. Chem. Biol. 5 (2001) 368-374). Human antibodies can also be produced in transgenic animals (e.g., mice) that are capable, upon immunization, of producing a full repertoire or a selection of human antibodies in the absence of endogenous immunoglobulin production. Transfer of the human germ-line immunoglobulin gene array in such germ-line mutant mice will result in the production of human antibodies upon antigen challenge (see, e.g., jakobovits, A., et al., Proc. Natl. Acad. Sci. USA 90 (1993) 2551-2555; jakobovits, A., et al., Nature 362 (1993) 255-258; Bruggemann, M., et al., Year Immunol. 7 (1993) 3340). Human antibodies can also be produced in phage display libraries (Hoogenboom, H. R., and Winter, G., J. Mol. Biol. 227 (1992) 381-388; Marks, J. D., et al., J. Mol. Biol. 222 (1991) 581-597).

The techniques of Cole et al. and Boerner et al. are also available for the preparation of human monoclonal antibodies (Cole et al., Monoclonal Antibodies and Cancer Therapy, Alan R. Liss, p. 77 (1985); and Boerner, P., et al., J. Immunol. 147 (1991) 86-95). Most preferably, however, human monoclonal antibodies are prepared by the method according to the present invention as described herein, which may be combined with improved EBV-B cell immortalization as described in Traggiai E, Becker S, Subbarao K, Kolesnikova L, Uematsu Y, Gismondo M R, Murphy B R, Rappuoli R, Lanzavecchia A. (2004): An efficient method to make human monoclonal antibodies from memory B cells: potent neutralization of SARS coronavirus. Nat Med. 10(8):871-5. The term “human antibody” as used herein also comprises such antibodies which are modified to generate the properties according to the invention as described herein.

Antibodies according to the present invention may be provided in purified form. Accordingly, the antibody according to the present invention, or the antibody fragment, may be a purified antibody or antibody fragment. Typically, the antibody will be present in a composition that is substantially free of other polypeptides e.g., where less than 90% (by weight), usually less than 60% and more usually less than 50% of the composition is made up of other polypeptides.

As used herein, the term “variable domain” (also referred to as “variable region”; variable domain of a light chain (VL), variable domain of a heavy chain (VH)) refers to the domain of an antibody, or antibody fragment, which is the N-terminal domain in classical naturally occurring antibodies, typically the domain providing the highest variability in classical naturally occurring antibodies, and which is involved directly in the binding of the antibody to the antigen. Typically, the domains of variable human light and heavy chains have the same general structure and each domain comprises framework (FR) regions whose sequences are widely conserved (in particular four framework (FR) regions) and three “hypervariable regions” or complementarity determining regions, CDRs (in particular three “hypervariable regions”/CDRs). The framework regions typically adopt a β-sheet conformation and the CDRs may form loops connecting the β-sheet structure. The CDRs in each chain are usually held in their three-dimensional structure by the framework regions and form together with the CDRs from the other chain the antigen binding site.

As used herein, the term “hypervariable region” refers to the amino acid residues of an antibody which are responsible for antigen-binding. The hypervariable region comprises the “complementarity determining regions” or “CDRs”. “Framework” or “FR” regions are those variable domain regions other than the hypervariable region residues as herein defined. CDR and FR regions may be determined according to the standard definition of Kabat et al., Sequences of Proteins of Immunological Interest, 5th ed., Public Health Service, National Institutes of Health, Bethesda, Md. (1991). Typically, in particular in native monospecific IgG antibodies, the three CDRs (CDR1, CDR2, and CDR3) are arranged non-consecutively in the variable domain. In other words, the CDRs on the heavy and/or light chain may be separated for example by framework regions, whereby a framework region (FR) is a region in the variable domain which is less “variable” than the CDR. For example, in an antibody a variable domain (or each variable domain, respectively) may preferably comprise four framework regions, separated by three CDRs. In particular, a variable domain of an antibody (light or heavy chain variable domain VH or VL) comprises from N- to C-terminus the domains FR1, CDR1, FR2, CDR2, FR3, CDR3, and FR4. CDRs on each chain are separated by such framework amino acids. Usually, the three CDRs of a heavy chain and the three CDRs of the connected light chain form together the antigen binding site (paratope). In other words, since in particular in native monospecific IgG antibodies antigen binding sites are typically composed of two variable domains, there are six CDRs for each antigen binding site (heavy chain: CDRH1, CDRH2, and CDRH3; light chain: CDRL1, CDRL2, and CDRL3). A single antibody, in particular a single native monospecific IgG antibody, usually has two (identical) antigen binding sites and therefore contains twelve CDRs (i.e. 2×six CDRs).

Due to their “multispecificity”, i.e. the different antigen binding sites, the heavy chain and/or the light chain of multispecific antibodies, or antigen binding fragments thereof, may (each) comprise more than three CDRs, in particular more than three different CDRs. For example, a multispecific antibody, or antigen binding fragments thereof, may comprise at least two different variable domains, wherein each of said at least two different variable domains is derived from a different monospecific antibody, e.g. of the IgG-type. Since such a monospecific antibody typically comprises three CDRs in the heavy chain and three CDRs in the light chain forming the antigen binding site, a multispecific antibody may in particular comprise three CDRs of a heavy chain of a first antibody and three CDRs of a light chain of a first antibody, three CDRs of a heavy chain of a second antibody and three CDRs of a light chain of a second antibody, optionally three CDRs of a heavy chain of a third antibody and three CDRs of a light chain of a third antibody etc. Thus, the number of CDRs comprised by a heavy chain and/or a light chain of a multispecific antibody is preferably a multiple of three, for example three, six, nine, twelve, etc. It is thereby also preferred that the sum of the CDRs comprised by both, heavy chain and light chain of a multispecific antibody is a multiple of six, for example six, twelve, eighteen etc. Since an “antigen binding site” is typically characterized by the CDRs, i.e. CDRH1, CDRH2, and CDRH3 as well as CDRL1, CDRL2, and CDRL3, it is preferred in multispecific antibodies that the CDRs are arranged such, that the order (e.g. CDRH1, CDRH2, and CDRH3 and/or CDRL1, CDRL2, and CDRL3 derived from the same monospecific antibody) is maintained to preserve the antigen binding site, i.e. to preserve to ability to specifically bind to a certain site in the antigen. This means that for example the order of CDRH1, CDRH2, and CDRH3 derived from a first monospecific antibody in an amino acid stretch is preferably not interrupted by any CDR derived from a second monospecific antibody. Importantly, if the multipecific antibody comprises antigen binding sites derived from at least two different monospecific antibodies, the CDRs or variable domains of these monospecific antibodies are arranged in the multipecific antibody such that the “antigen receptor” of each monospecific antibody from which the CDRs (or variable regions) are derived, is preserved, i.e. its ability to specifically bind to a certain site in the antigen, is preserved.

In the context of the present invention, a variable domain may be any variable domain (in particular, VH and/or VL) of a naturally occurring antibody or a variable domain may be a modified/engineered variable domain. Modified/engineered variable domains are known in the art. Typically, variable domains are modified/engineered to delete or add one or more functions, e.g., by “germlining” somatic mutations (“removing” somatic mutations) or by humanizing.

As used herein, the term “constant domains” refers to domains of an antibody which are not involved directly in binding an antibody to an antigen, but exhibit various effector functions. Typically, a heavy chain comprises three or four constant domains, depending on the immunoglobulin class: CH1, CH2, CH3, and, optionally, CH4 (in N—C-terminal direction). Accordingly, the constant region of a heavy chain is typically formed (in N- to C-terminal direction) by: CH1-hinge (flexible polypeptide comprising the amino acids between the first and second constant domains of the heavy chain)-CH2-CH3 (-CH4). A light chain typically comprises only one single constant domain, referred to as CL, which typically also forms the constant region of the light chain. In the context of the present invention, a constant domain may be any constant domain (in particular, CL, CH1, CH2, CH3 and/or CH4) of a naturally occurring antibody or a constant domain may be a modified/engineered constant domain. Modified/engineered constant domains are known in the art. Typically, constant domains are modified/engineered to delete or add one or more functions, e.g., in the context of the functionality of the Fc region. Depending on the amino acid sequence of the constant region of their heavy chains, antibodies or immunoglobulins are divided in the classes: IgA, IgD, IgE, IgG and IgM, and several of these may be further divided into subclasses, e.g. IgG1, IgG2, IgG3, and IgG4, IgA1 and IgA2. The heavy chain constant regions that correspond to the different classes of immunoglobulins are called α, ε, γ, and μ, respectively. The antibodies according to the invention are preferably of IgM type or IgG type. Unlike IgG, IgM does not contain a hinge region but does contain an additional constant domain and an 18 amino acid tailpiece at the carboxy terminus, which contains a cysteine and is involved in multimerisation of the molecule.

In general, antibodies can be of any isotype (e.g., IgA, IgG, IgM i.e. an α, γ or μ heavy chain), but will preferably be IgM or IgG. Within the IgG isotype, antibodies may be IgG1, IgG2, IgG3 or IgG4 subclass, whereby IgG1 is preferred. Antibodies may have a κ or a λ light chain.

As used herein, the term “recombinant antibody” is intended to include all antibodies, which do not occur in nature, for example antibodies produced by B cells engineered according to the method of the present invention.

As used herein, the term “multispecific” in the context of an antibody, or an antibody fragment, refers to the ability of the antibody or the antibody fragment to bind to at least two different epitopes, e.g. on different antigens or on the same antigen. Thus, terms like “bispecific”, trispecific”, “tetraspecific” etc. refer to the number of different epitopes to which the antibody can bind to. For example, conventional monospecific IgG-type antibodies have two identical antigen binding sites (paratopes) and can, thus, only bind to identical epitopes (but not to different epitopes). A multispecific antibody, in contrast, has at least two different types of paratopes/binding sites and can, thus, bind to at least two different epitopes. As used herein, “paratope” refers to an antigen binding site of the antibody. Moreover, a single “specificity” may refer to one, two, three or more identical paratopes in a single antibody (the actual number of paratopes/binding sites in one single antibody molecule is referred to as “valency”). For example, a single native IgG antibody is monospecific and bivalent, since it has two identical paratopes. Accordingly, a multispecific antibody comprises at least two (distinct) paratopes/binding sites. Thus, the term “multispecific antibodies” refers to antibodies having more than one paratope and the ability to bind to two or more different epitopes. The term “multispecific antibodies” comprises in particular bispecific antibodies as defined above, but typically also protein, e.g. antibody, scaffolds, which bind in particular to three or more distinct epitopes, i.e. antibodies with three or more paratopes/binding sites.

In particular, the multispecific antibody, or the antibody fragment, may comprise two or more paratopes/binding sites, wherein some paratopes/binding sites may be identical so that all paratopes/binding sites of the antibody belong to at least two different types of paratopes/binding sites and, hence, the antibody has at least two specificities. For example, the multispecific antibody or antibody fragment may comprise four paratopes/binding sites, wherein each two paratopes/binding sites are identical (i.e. have the same specificity) and, thus, the antibody or fragment thereof is bispecific and tetravalent (two identical paratopes/binding sites for each of the two specificities). Thus, “one specificity” refers in particular to one or more paratopes/binding sites exhibiting the same specificity (which typically means that such one or more paratopes/binding sites are identical) and, thus, “two specificities” may be realized by two, three, four five, six or more paratopes/binding sites as long as they refer to only two specificities. Alternatively, a multispecific antibody may comprise one single paratope/binding site for each (of the at least two) specificity, i.e. the multispecific antibody comprises in total at least two paratopes/binding sites. For example, a bispecific antibody comprises one single paratope/binding site for each of the two specificities, i.e. the antibody comprises in total two paratopes/binding sites. It is also preferred that the antibody comprises two (identical) paratopes/binding sites for each of the two specificities, i.e. the antibody comprises in total four paratopes/binding sites. Preferably the antibody comprises three (identical) paratopes/binding sites for each of the two specificities, i.e. the antibody comprises in total six paratopes/binding sites.

As used herein, the term “antigen” refers to any structural substance which serves as a target for the receptors of an adaptive immune response, in particular as a target for antibodies, T cell receptors, and/or B cell receptors. An “epitope”, also known as “antigenic determinant”, is the part (or fragment) of an antigen that is recognized by the immune system, in particular by antibodies, T cell receptors, and/or B cell receptors. Thus, one antigen has at least one epitope, i.e. a single antigen has one or more epitopes. An antigen may be (i) a peptide, a polypeptide, or a protein, (ii) a polysaccharide, (iii) a lipid, (iv) a lipoprotein or a lipopeptide, (v) a glycolipid, (vi) a nucleic acid, or (vii) a small molecule drug or a toxin. Thus, an antigen may be a peptide, a protein, a polysaccharide, a lipid, a combination thereof including lipoproteins and glycolipids, a nucleic acid (e.g. DNA, siRNA, shRNA, antisense oligonucleotides, decoy DNA, plasmid), or a small molecule drug (e.g. cyclosporine A, paclitaxel, doxorubicin, methotrexate, 5-aminolevulinic acid), or any combination thereof. Preferably, the antigen is selected from (i) a peptide, a polypeptide, or a protein, (ii) a polysaccharide, (iii) a lipid, (iv) a lipoprotein or a lipopeptide and (v) a glycolipid; more preferably, the antigen is a peptide, a polypeptide, or a protein.

The term “antigen binding site” as used herein refers to the part of the antibody which comprises the area which specifically binds to and is complementary to part or all of an antigen. Where an antigen is large, an antibody may only bind to a particular part of the antigen, which part is termed “epitope”. Typically, two variable domains, in particular a heavy chain variable domain VH and a light chain variable domain VL, associate to form one an antigen binding site. In particular, the antigen binding site is formed by the three CDRs of the heavy chain variable domain and by the three CDRs of the light chain variable domain together, i.e. by six CDRs, as described above.

The term “specifically binding” and similar reference does not encompass non-specific sticking.

The term “linker” (also referred to as “spacer”), as used herein, refers to a peptide adapted to connect distinct domains of a polypeptide or protein, such as an antibody or an antibody fragment. Linkers are known in the art and described in detail, e.g. in Reddy Chichili V P, Kumar V, Sivaraman J. Linkers in the structural biology of protein-protein interactions. Protein Science: A Publication of the Protein Society. 2013; 22(2):153-167). Typically, linkers are designed such that they do not affect functionality. In particular, a linker does not specifically bind to a target. A linker may contain any amino acids, the amino acids glycine (G) and serine (S) may be preferred. Preferably, the linker is composed of the amino acids glycine (G) and serine (S) (“GS-linker”). If two or more linkers occur in one polypeptide or protein, the linkers may be equal or differ from each other. Furthermore, the linker may have a length of 1, 2, 3, 4, 5, 6, 7, 8, 9, 10, 11, 12, 13, 14, 15, 16, 17, 18, 19 or 20 amino acids.

As used herein, the term “nucleic acid or nucleic acid molecule” is intended to include DNA molecules and RNA molecules. A nucleic acid molecule may be single-stranded (ss) or double-stranded (ds).

As used herein, the terms “cell,” “cell line,” and “cell culture” are used interchangeably and all such designations include progeny. Thus, the words “transformants” and “transformed cells” include the primary subject cell and cultures derived therefrom without regard for the number of transfers. It is also understood that all progeny may not be precisely identical in DNA content, due to deliberate or inadvertent mutations. Variant progeny that have the same function or biological activity as screened for in the originally transformed cell are included. Where distinct designations are intended, it will be clear from the context.

As used herein, “sequence variant” (also referred to as “variant”) refers to any alteration in a reference sequence, whereby a reference sequence is any of the sequences listed in the “Tables of Sequences and SEQ ID Numbers” (sequence listing), i.e. SEQ ID NO: 1 to SEQ ID NO: 115. Thus, the term “sequence variant” includes nucleotide sequence variants and amino acid sequence variants. Of note, the sequence variants referred to herein are in particular functional sequence variants, i.e. sequence variants maintaining the biological function of the reference sequence. For example, the functionality of the (poly)peptide of interest (having, for example, a binding functionality) the intronic sequence (e.g. having a splice site functionality and/or a splicing enhancer functionality) may be maintained.

Preferred sequence variants are thus (functional) sequence variants having at least 70%, at least 75%, at least 80%, at least 85%, at least 88%, at least 90%, at least 92%, at least 95%, at least 96%, at least 97%, at least 98% or at least 99% sequence identity to a reference sequence. The phrase “sequence variant thereof having at least 70%, at least 75%, at least 80%, at least 85%, at least 88%, at least 90%, at least 92%, at least 95%, at least 96%, at least 97%, at least 98% or at least 99% sequence identity”, as used herein, means the higher the % sequence identity, the more preferred the sequence variant. In other words, the phrase “sequence variant thereof having at least 70%, at least 75%, at least 80%, at least 85%, at least 88%, at least 90%, at least 92%, at least 95%, at least 96%, at least 97%, at least 98% or at least 99% sequence identity”, means in particular that the sequence variant has at least 70% sequence identity, preferably at least 75% sequence identity, preferably at least 80% sequence identity, more preferably at least 85% sequence identity, more preferably at least 88% sequence identity, even more preferably at least 90% sequence identity, even more preferably at least 92% sequence identity, still more preferably at least 95% sequence identity, still more preferably at least 96% sequence identity, particularly preferably at least 97% sequence identity, particularly preferably at least 98% sequence identity and most preferably at least 99% sequence identity to the respective reference sequence.

Sequence identity is usually calculated with regard to the full length of the reference sequence (i.e. the sequence recited in the application). Percentage identity, as referred to herein, can be determined, for example, using BLAST using the default parameters specified by the NCBI (the National Center for Biotechnology Information; http://www.ncbi.nlm.nih.gov/) [Blosum 62 matrix; gap open penalty=11 and gap extension penalty=1].

As used herein, a “nucleotide sequence variant” has an altered sequence in which one or more of the nucleotides in the reference sequence is deleted, or substituted, or one or more nucleotides are inserted into the sequence of the reference nucleotide sequence. Nucleotides are referred to herein by the standard one-letter designation (A, C, G, or T). Due to the degeneracy of the genetic code, a “nucleotide sequence variant” can either result in a change in the respective reference amino acid sequence, i.e. in an “amino acid sequence variant” or not. Preferred sequence variants are such nucleotide sequence variants, which do not result in amino acid sequence variants (silent mutations), but other non-silent mutations are within the scope as well, in particular mutant nucleotide sequences, which result in an amino acid sequence, which may be at least 80%, preferably at least 90%, more preferably at least 95% sequence identical to the reference sequence.

An “amino acid sequence variant” has an altered sequence in which one or more of the amino acids in the reference sequence is deleted or substituted, or one or more amino acids are inserted into the sequence of the reference amino acid sequence. As a result of the alterations, the amino acid sequence variant has an amino acid sequence which is at least 80% identical to the reference sequence, preferably, at least 90% identical, more preferably at least 95% identical, most preferably at least 99% identical to the reference sequence. Variant sequences which are at least 90% identical have no more than 10 alterations, i.e. any combination of deletions, insertions or substitutions, per 100 amino acids of the reference sequence.

While it is possible to have non-conservative amino acid substitutions, it is preferred that the substitutions be conservative amino acid substitutions, in which the substituted amino acid has similar structural or chemical properties with the corresponding amino acid in the reference sequence. By way of example, conservative amino acid substitutions involve substitution of one aliphatic or hydrophobic amino acids, e.g. alanine, valine, leucine and isoleucine, with another; substitution of one hydoxyl-containing amino acid, e.g. serine and threonine, with another; substitution of one acidic residue, e.g. glutamic acid or aspartic acid, with another; replacement of one amide-containing residue, e.g. asparagine and glutamine, with another; replacement of one aromatic residue, e.g. phenylalanine and tyrosine, with another; replacement of one basic residue, e.g. lysine, arginine and histidine, with another; and replacement of one small amino acid, e.g., alanine, serine, threonine, methionine, and glycine, with another.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include the fusion to the N- or C-terminus of an amino acid sequence to a reporter molecule or an enzyme.

Importantly, the alterations in the sequence variants do not abolish the functionality of the respective reference sequence, in the present case, e.g., the functionality of a sequence of an antibody, or antibody fragment, to bind to its antigens and/or the additional functionality provided by the functional domain, for example to bind to a target of an (independent) binding site. Guidance in determining which nucleotides and amino acid residues, respectively, may be substituted, inserted or deleted without abolishing such functionality are found by using computer programs well known in the art.

As used herein, a nucleic acid sequence or an amino acid sequence “derived from” a designated nucleic acid, peptide, polypeptide or protein refers to the origin of the nucleic acid, peptide, polypeptide or protein. Preferably, the nucleic acid sequence or amino acid sequence which is derived from a particular sequence has an amino acid sequence that is essentially identical to that sequence or a portion thereof, from which it is derived, whereby “essentially identical” includes sequence variants as defined above. Preferably, the nucleic acid sequence or amino acid sequence which is derived from a particular peptide or protein, is derived from the corresponding domain in the particular peptide or protein. Thereby, “corresponding” refers in particular to the same functionality. For example, an “extracellular domain” corresponds to another “extracellular domain” (of another protein), or a “transmembrane domain” corresponds to another “transmembrane domain” (of another protein). “Corresponding” parts of peptides, proteins and nucleic acids are thus easily identifiable to one of ordinary skill in the art. Likewise, sequences “derived from” other sequence are usually easily identifiable to one of ordinary skill in the art as having its origin in the sequence.

Preferably, a nucleic acid sequence or an amino acid sequence derived from another nucleic acid, peptide, polypeptide or protein may be identical to the starting nucleic acid, peptide, polypeptide or protein (from which it is derived). However, a nucleic acid sequence or an amino acid sequence derived from another nucleic acid, peptide, polypeptide or protein may also have one or more mutations relative to the starting nucleic acid, peptide, polypeptide or protein (from which it is derived), in particular a nucleic acid sequence or an amino acid sequence derived from another nucleic acid, peptide, polypeptide or protein may be a functional sequence variant as described above of the starting nucleic acid, peptide, polypeptide or protein (from which it is derived). For example, in a peptide/protein one or more amino acid residues may be substituted with other amino acid residues or one or more amino acid residues may be inserted or deleted.

As used herein, the term “mutation” relates to a change in the nucleic acid sequence and/or in the amino acid sequence in comparison to a reference sequence, e.g. a corresponding genomic sequence. A mutation (e.g. in comparison to a genomic sequence) may be, for example, a (naturally occurring) somatic mutation, a spontaneous mutation, an induced mutation, e.g. induced by enzymes, chemicals or radiation, or a mutation obtained by site-directed mutagenesis (molecular biology methods for making specific and intentional changes in the nucleic acid sequence and/or in the amino acid sequence). Thus, the terms “mutation” or “mutating” shall be understood to also include physically making a mutation, e.g. in a nucleic acid sequence or in an amino acid sequence. A mutation includes substitution, deletion and insertion of one or more nucleotides or amino acids as well as inversion of several (two or more) successive nucleotides or amino acids. To achieve a mutation in an amino acid sequence, preferably a mutation may be introduced into the nucleotide sequence encoding said amino acid sequence in order to express a (recombinant) mutated polypeptide. A mutation may be achieved e.g., by altering, e.g., by site-directed mutagenesis, a codon of a nucleic acid molecule encoding one amino acid to result in a codon encoding a different amino acid, or by synthesizing a sequence variant, e.g., by knowing the nucleotide sequence of a nucleic acid molecule encoding a polypeptide and by designing the synthesis of a nucleic acid molecule comprising a nucleotide sequence encoding a variant of the polypeptide without the need for mutating one or more nucleotides of a nucleic acid molecule.

As used herein, the terms “upstream” and “downstream” both refer to relative positions in DNA or RNA. Each strand of DNA or RNA has a 5′ end and a 3′ end (named for the carbon position on the deoxyribose or ribose ring). By convention, upstream and downstream relate to the 5′ to 3′ direction in which RNA transcription takes place. Upstream is toward the 5′ end of the RNA molecule and downstream is toward the 3′ end. In double-stranded DNA, upstream is toward the 5′ end of the coding strand for the exon of interest and downstream is toward the 3′ end. Due to the anti-parallel nature of DNA, this means the 3′ end of the template strand is upstream of the gene and the 5′ end is downstream.

The term “disease” as used in the context of the present invention is intended to be generally synonymous, and is used interchangeably with, the terms “disorder” and “condition” (as in medical condition), in that all reflect an abnormal/pathologic condition of the human or animal body or of one of its parts that typically impairs normal functioning, is typically manifested by distinguishing signs and symptoms, and usually causes the human or animal to have a reduced duration or quality of life.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention.

It is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

Method for Editing the Genome of a B Lymphocyte

In a first aspect the present invention provides a method for editing the genome of an isolated B lymphocyte comprising the following steps:
(i) activating endogenous activation-induced cytidine deaminase of the B lymphocyte; and
(ii) introducing a DNA molecule comprising a nucleotide sequence encoding a (poly)peptide of interest into the B lymphocyte.

“Editing the genome” means to insert, delete, or alter a (naturally occurring) gene or gene locus of interest, in particular to produce a B cell with altered specificity and/or function. Accordingly, by the method of the present invention an engineered B lymphocyte can be obtained, wherein the genome of the B lymphocyte comprises the nucleotide sequence encoding the (poly)peptide of interest. Preferably, the gene locus of interest, which is edited according to the present invention, is an immunoglobulin gene locus, i.e. a gene locus encoding an immunoglobulin (antibody) polypeptide chain. Preferably, the method of the invention provides engineered B cells (in which an immunoglobulin gene locus was edited) to produce (and secrete) recombinant (customized) antibodies. Accordingly, the present invention preferably provides a method for editing an immunoglobulin gene locus in the genome of an isolated B cell comprising the steps as described herein.

The terms “B lymphocyte” and “B cell” are used interchangeably. In general, a “B lymphocyte” is a type of white blood cells of the lymphocyte subtype. A major function of a B lymphocyte is to secrete antibodies. Accordingly, B lymphocytes belong to the humoral component of the adaptive immune system. In addition, B lymphocytes can present antigens and secrete cytokines. In contrast to the other two classes of lymphocytes, T cells and natural killer cells, B lymphocytes express B cell receptors (BCRs) on their cell membrane. BCRs allow the B cell to bind to a specific antigen, against which it will initiate an antibody response.

In general, the B lymphocyte may be of any species. In some embodiments, the B lymphocyte is a mammalian B lymphocyte. Preferably, the B lymphocyte is a human B lymphocyte. Accordingly, in some embodiments the B lymphocyte is not a chicken B lymphocyte or a murine B lymphocyte. In particular, the IgL locus of the B lymphocyte is preferably not deleted.

In general, the term “isolated” B lymphocyte refers to a B lymphocyte, which is not part of a human or animal body. In particular, the isolated B lymphocyte may be a primary B lymphocyte or (a B lymphocyte of) a B cell line.

A cell line is typically continuous (i.e., it can proliferate indefinitely), in particular due to tumor or artificial immortalization, e.g. Epstein-Barr virus (EBV)-immortalization. B cell lines are commercially available, for example Ramos (ATCC®-CRL-1596) or SKW 6.4 (ATCC® TIB-125). In particular, a B lymphocyte of the B cell line has the capacity that its endogenous activation-induced cytidine deaminase (AID) can be activated. Alternatively, a B lymphocyte of the B cell line may have constitutive activity of its endogenous activation-induced cytidine deaminase (AID), such as the RAMOS B cell line (e.g., Ramos RA1 (ATCC®-CRL-1596)).

Most preferably, the isolated B lymphocyte is a primary B lymphocyte. “Primary” B lymphocytes are isolated from living tissue and established for in vitro culture. In contrast to continuous (tumor or artificially immortalized) cell lines, “primary” cells are “freshly” isolated, i.e. they have undergone only very few cell divisions in vitro. Typically, primary cells have a finite life span, i.e. they are not “immortalized” like cell lines. In particular, primary cells have not been modified in any way (except for enzymatic and/or physical dissociation required to extract the cells from their tissue of origin).

Primary B cells can be isolated from blood or from lymphoid tissue, such as bone marrow, thymus, spleen and/or lymph nodes. Typically, B cells are isolated from a (isolated) sample of a subject. The sample is for example blood or lymphoid tissue, such as bone marrow, thymus, spleen and/or lymph nodes. For example, the B cell is isolated from peripheral blood mononuclear cells (PBMCs), bone marrow, or the spleen.

Methods for isolating primary B lymphocytes are known in the art. For example, B cells may be isolated by flow cytometry, magnetic cell isolation and cell separation (MACS), RosetteSep, or antibody panning. One or more isolation techniques may be utilized in order to provide isolated B lymphocytes with sufficient purity, viability, and yield. Preferably, primary B cells are isolated by MACS or RosetteSep. For example, B cells may be isolated, in particular from peripheral blood mononuclear cells (PBMCs), by magnetic cell sorting. To this end, for example anti-CD19 microbeads may be used, e.g. as available from Miltenyi Biotec.

Preferably, the purity of the isolated (primary) B lymphocytes is at least about 80%, 85%, 90%, 91%, 92%, 93%, 94%, 95%, or more. Moreover, it is preferred that the isolated (primary) B cells are at least about 70%, 75%, 80%, 85%, 90%, 95% or more viable. Optionally, after isolation, primary B lymphocytes may be expanded in culture.

In a preferred embodiment, the primary B lymphocytes are isolated from a (isolated sample of a) patient. After engineering, the B cells may be administered to the same patient (from which they or their progenitor cells were isolated). Such B cells may be referred to as “autologous” B cells. In this way, a patient can produce customized antibodies in vivo (“by himself”). Alternatively, the engineered B cells may be used to establish a (immortalized) B cell line, e.g. for in vitro production of customized antibodies.

In a first step of genome editing of the isolated B lymphocyte, an endogenous activation-induced cytidine deaminase of the B lymphocyte is activated. Various ways to activate activation-induced cytidine deaminase of a B lymphocyte are known in the art and described herein below in more detail. Thereby, double strand breaks (DSBs) in the genomic DNA are induced, in particular in the switch region of an immunoglobulin gene locus. In general, activation-induced cytidine deaminase (also known as “AID” or “AICDA”) is an enzyme, which creates mutations in DNA by deamination of a cytosine base, thereby turning cytosine into uracil. AID is recognized as “master regulator” of secondary antibody diversification, as it mediates somatic hypermutation (SHM), class switch recombination (CSR) and gene conversion (GC). In particular, AID mediates DNA cleavage of the switch region (also referred to as “S region”) in an immunoglobulin gene locus in CSR. A switch region is a region of repetitive DNA sequences in an immunoglobulin gene locus, in particular located upstream of a CH gene portion.

CSR occurs between two switch regions, located upstream of each CH gene portion, excising the intervening DNA (“switch circle”) and juxtaposing the variable region with the downstream CH gene portion. AID effects deamination of cytosine, resulting in dU, which is then removed by the combined action of uracil-N-glycosylase (UNG) and the apyrimidinic endonuclease (APE1), causing a single-strand DNA break (SSB). When SSBs are in close proximity on opposite DNA strands, double-strand breaks (DSBs) are formed. DSBs may also be formed through the action of the mismatch repair (MMR) pathway by “end processing”. After the formation of DSBs in two switch regions, the remaining “ends” of the DNA are joined and recombination occurs through the classical non-homologous end-joining (C-NHEJ) or alternative end-joining (A-EJ) pathways.

The term “endogenous” means that the activation-induced cytidine deaminase originates from within the B lymphocyte. In particular, the activation-induced cytidine deaminase is expressed on the basis of the endogenous gene encoding the activation-induced cytidine deaminase (i.e., not by means of a construct introduced into the B cell). Accordingly, the activation-induced cytidine deaminase is expressed by the B lymphocyte. Accordingly, there is no need to introduce an (exogenous) nuclease (or a nucleic acid, vector or virus encoding or expressing such a nuclease) into the B cell. Rather, the breaks in the genomic DNA are performed by the B cell's own machinery, which is only activated according to the present invention. In particular, the method of the invention does typically not involve the introduction of a nuclease (or a nucleic acid encoding a nuclease or other exogenous means for encoding and/or expression of a nuclease) into the B cell.

After activation of the B lymphocyte's activation-induced cytidine deaminase, a DNA molecule comprising a nucleotide sequence encoding a (poly)peptide of interest is introduced into the B cell in a further step (step (ii)) of the method according to the present invention. Accordingly, step (ii) of the method according to the present invention is typically performed after step (i). In particular, the endogenous activation-induced cytidine deaminase of the B cell is activated before a DNA molecule comprising a nucleotide sequence encoding a (poly)peptide of interest is introduced into the B cell.

Accordingly, the B lymphocyte is transfected with a DNA molecule comprising a nucleotide sequence encoding a (poly)peptide of interest. In general, the term “transfection” refers to the introduction of nucleic acid molecules, such as DNA or RNA molecules, into cells, preferably into eukaryotic cells. In the context of the present invention, the term “transfection” encompasses any method known to the skilled person for introducing a DNA molecule into a B lymphocyte. Such methods encompass, for example, viral and non-viral methods of transfection. Viruses which may be used for gene transfer include retrovirus (including lentivirus), herpes simplex virus, adenovirus and adeno-associated virus (AAV). However, in some embodiments the B lymphocyte is not transduced with a retrovirus. Moreover, nanoparticles may also be used for transfection. Further non-viral transfection methods include many chemical and physical methods. Chemical transfection methods include lipofection, e.g. based on cationic lipids and/or liposomes, calcium phosphate precipitation, or transfection based on cationic polymers, such as DEAE-dextran or polyethylenimine (PEI) etc. Physical transfection methods include electroporation, ballistic gene transfer (introduces particles coated with DNA into cells), microinjection (DNA transfer through microcapillaries into cells), and nucleofection. Preferably, the introduction of the DNA molecule comprising a nucleotide sequence encoding a (poly)peptide of interest into the B lymphocyte is non-viral. Most preferably, the DNA is introduced by nucleofection.

A (poly)peptide of interest may be any (poly)peptide, which is envisaged to be expressed as a (part of) a customized antibody. Preferred (poly)peptides of interest are described herein below in more detail.

The method for editing the genome of an isolated B lymphocyte according to the present invention is shown schematically in FIG. 1. Without being bound to any theory, the present inventors believe that integration of the DNA molecule comprising a nucleotide sequence encoding the (poly)peptide of interest in the B cell genome, in particular in the switch region of an immunoglobulin gene locus of the B lymphocyte, occurs by natural mechanisms, such as homologous recombination (HR), nonhomologous end-joining (c-NHEJ) or alternative end-joining (A-EJ) pathways. In other words, after introduction (transfection) of the DNA molecule comprising a nucleotide sequence encoding a (poly)peptide of interest into the B lymphocyte, the B lymphocyte's endogenous repair mechanisms subsequently repairs the induced break(s) by natural processes, such as homologous recombination (HR), nonhomologous end-joining (NHEJ) and/or or alternative end-joining (A-EJ), thereby integrating the DNA molecule comprising a nucleotide sequence encoding a (poly)peptide of interest into the genome of the B lymphocyte. In c-NHEJ and A-EJ pathways, DNA breaks are detected by the Mre11/Rad50/Nbs1(MRN) and ataxia telangiectasia mutated (ATM) complexes. In c-NHEJ Ku protein Ku70 and Ku80 heterodimers and DNA-dependent protein kinase (DNAPKs) form a scaffold holding the non-homologous DNA ends together, modifying the DNA ends and recruiting DNA ligase IV to rejoin the DNA ends. A-EJ, in contrast, occurs independently of Ku and DNA ligase IV and possibly utilizes PARP and/or CtIP as scaffolds for additional factors required for DNA end processing and ligation via DNA ligase 1 or III.

In summary, compared to methods of the prior art for engineering B cells, which depend on administration of an exogenous (engineered) nuclease, the method for editing the genome of a B lymphocyte according to the present invention lowers the risk for undesired off-target mutations. In particular, in the method according to the present invention genome editing of a B lymphocyte i) can be performed as early as 1 day after isolation and B cell stimulation, and ii) works without addition of an engineered nuclease, such as Cas9.

In particular, the method according to the present invention does not involve an exogenous nuclease. In other words, in the method according to the present invention in particular presence of an exogenous nuclease is not required. Accordingly, it is preferred in the context of the present invention that neither an exogenous nuclease itself nor a nucleic acid encoding an exogenous nuclease is introduced into the B lymphocyte. In general, a nuclease is an enzyme capable of cleaving the phosphodiester bonds between monomers of nucleic acids. An exogenous nuclease is a nuclease, which does not originate within the B lymphocyte, in particular a nuclease, which is not expressed by the B cell. More preferably, the method according to the present invention does not involve/utilize a CRISPR nuclease, a zinc finger nuclease, a transcription activator-like nuclease or a meganuclease.

Accordingly, it is preferred that the method according to the present invention does not involve an artificially engineered nuclease. Such engineered nucleases are often referred to as “molecular scissors” and include engineered meganucleases (e.g., engineered meganuclease re-engineered homing endoncucleases), transcription activator-like nucleases (TALENs), zinc finger nucleases (ZFNs) and RNA-guided nucleases, such as CRISPR (clustered regularly interspaced short palindromic repeats) nucleases, such as a Cas nuclease (e.g., Cas9), a Cpf1 nuclease, a Cmr nuclease, a Csf nuclease, a Csm nuclease, a Csn nuclease, a Csy nuclease, a C2cl nuclease, a C2c3 nuclease, or a C2c3 nuclease. More preferably, the method according to the present invention does not involve/utilize an engineered nuclease, such as a CRISPR nuclease, a zinc finger nuclease, a transcription activator-like nuclease or a meganuclease.

Meganucleases are endonucleases characterized by a large recognition site of 12 to 40 base pairs. Engineered meganucleases are often derived from homing endonucleases. Zinc-finger nucleases (ZFNs) are artificial restriction enzymes generated by fusing a zinc finger DNA-binding domain to a DNA-cleavage domain, wherein the zinc finger domains may be engineered to target specific desired DNA sequences which enables zinc-finger nucleases to target unique sequences within complex genomes. Transcription activator-like effector nucleases (TALEN) are restriction enzymes made by fusing a TAL effector DNA-binding domain to a DNA cleavage domain, which can be engineered to cut specific sequences of DNA. CRISPR is a family of DNA sequences in bacteria and archaea containing snippets of DNA from viruses that have attacked the prokaryote, which are used by the prokaryote to detect and destroy DNA from similar viruses during subsequent attacks. RNA harboring the spacer sequence in the CRISPR sequence helps CRISPR nucleases recognize and cut exogenous DNA. For gene editing, usually a CRISPR nuclease, such as a Cas nuclease (e.g., Cas9), a Cpf1 nuclease, a Cmr nuclease, a Csf nuclease, a Csm nuclease, a Csn nuclease, a Csy nuclease, a C2c1 nuclease, a C2c3 nuclease, or a C2c3 nuclease, is introduced into a cell together with guide RNA to cur the cell's genome at a desired location.

Moreover, the present method does preferably not involve the introduction of any guide nucleic acid (guide RNA or guide DNA) into the B lymphocyte. In addition to the above mentioned CRISPR/Cas system, which uses guide RNA also other genome editing methods using guide nucleic acids are known in the art, for example a method based on the argonaute (Ago) nuclease, which uses 5′ phosphorylated short single strand nucleic acids (RNA or DNA) as guide to cleave a target. However, the method according to the present invention utilizes activation-induced cytidine deaminase (AID), which naturally targets the switch region and, thus, no guide nucleic acids are required and, in particular, no guide nucleic acids are involved.

Optionally, the method according to the present invention may further comprise a step:
(iii) confirming integration of the nucleotide sequence encoding the (poly)peptide of interest into the genome of the B lymphocyte.

In particular, this step (iii) is performed after steps (i) and (ii) as described above. Step (iii) may be performed, for example, by sequencing (nucleic acids, such as genomic DNA, from the B lymphocyte) and/or by checking whether the B cell receptor or antibodies produced by the engineered B lymphocyte contain the (poly)peptide of interest. For example, if the (poly)peptide of interest is a specific binding site, binding of antibodies produced by the engineered B lymphocyte to the specific binding partner may be assessed. Successful integration of the (poly)peptide of interest into the antibody may also be assessed by cell-surface staining with fluorescently labeled antibodies and flow cytometry analysis, for example if the integrated (poly)peptide of interest is not part of a surface molecule, which is endogenously expressed by B cells. Furthermore, integration of the nucleotide sequence encoding the (poly)peptide of interest into the genome of the B lymphocyte may be validated by PCR amplification and/or (subsequent) sequencing of the immunoglobulin switch region including switch-μ, and, optionally, the corresponding regions of all alpha and gamma isotypes.

DNA Molecule Comprising a Nucleotide Sequence Encoding a (Poly)Peptide of Interest

The DNA molecule introduced into the B lymphocyte in step (ii) may be of any form, e.g. a circular (such as a plasmid) or a linear DNA molecule. For example, if a circular DNA molecule (plasmid) is introduced in step (ii), it may contain at least one restriction site for endogenous DNase, such that the plasmid can be cleaved for integration into the genome. Preferably, the DNA molecule introduced into the B lymphocyte in step (ii) of the method according to the invention is a linear or linearized DNA molecule. For example, the DNA molecule may be prepared as plasmid (or as component of a plasmid), which is cleaved (such that it comprises a nucleotide sequence encoding the (poly)peptide of interest) before it is introduced into the B lymphocyte (linearized DNA molecule).

Moreover, the DNA molecule introduced into the B lymphocyte in step (ii) may be a single strand DNA molecule (ssDNA) or a double strand DNA molecule (dsDNA). As shown in Example 1, both, ssDNA and dsDNA can be used in the method according to the present invention. Preferably, the DNA molecule is a dsDNA molecule.

Moreover, a double strand DNA molecule introduced into the B lymphocyte in step (ii) may have a blunt end, a 5′ and/or 3′ overhang, or a frayed end. A “blunt end” is the simplest end of a double stranded DNA molecule, in which both strands of the DNA molecule terminate in a base pair of complementary bases (adenine: thymidine (A: T) and cytosine: guanine (C: G), respectively). In other words, in a blunt end, each nucleotide of the first strand is paired with a complementary nucleotide of the other strand (forming a base pair). In contrast, an “overhang” is a stretch of unpaired nucleotides in the end of a dsDNA molecule. These unpaired nucleotides can be in either strand, creating either 3′ or 5′ overhangs. Overhangs of at least two unpaired nucleotides are also referred to as “sticky ends” or “cohesive ends”. Accordingly, a sticky or cohesive end has a protruding single strand with unpaired nucleotides (called overhang)—whereas blunt ends do not have protruding strands. Finally, a “frayed end” refers to a region of a dsDNA molecule near the end with a significant proportion of non-complementary sequences (in non-complementary “base pairs”). However, the incorrectly matched nucleotides tend to avoid bonding, thus appearing similar to the strands in a fraying piece of rope.

Preferably, the DNA molecule has sticky ends or blunt ends. Most preferably, the DNA molecule has blunt ends.

It is also preferred that the DNA molecule, or at least the nucleotide sequence of the DNA molecule, which encodes the (poly)peptide of interest, is codon-optimized. In particular, nucleotide sequences encoding a (poly)peptide of interest, wherein the (poly)peptide of interest is not of human origin, are preferably codon-optimized. In general, codon-optimization can improve translation and expression of a recombinant protein in human cells. Various methods for codon-optimization are known in the art. For example, computational tools, such as JCat (Grote A, Hiller K, Scheer M, Munch R, Nortemann B, Hempel D C, Jahn D. JCat: a novel tool to adapt codon usage of a target gene to its potential expression host. Nucleic Acids Res. 2005 Jul. 1; 33(Web Server issue):W526-31), Synthetic Gene Designer (Wu G, Bashir-Bello N, Freeland S J. The Synthetic Gene Designer: a flexible web platform to explore sequence manipulation for heterologous expression. Protein Expr Purif. 2006 June; 47(2):441-5) and OPTIMIZER (Puigbò P, Guzmán E, Romeu A, Garcia-Vallvé S. OPTIMIZER: a web server for optimizing the codon usage of DNA sequences. Nucleic Acids Res. 2007 July; 35(Web Server issue):W126-31) may be used, which were developed to quantify and optimize the codon usage frequency of the coding sequence in terms of the host's codon adaptation index (CAI) or individual codon usage (ICU). Moreover, optimization of codon pairing, also known as codon context (CC), may be used, e.g. for improving heterologous gene expression (for example as described in: Chung B K, Yusufi F N, Mariati, Yang Y, Lee D Y. Enhanced expression of codon optimized interferon gamma in CHO cells. J Biotechnol. 2013 Sep. 10; 167(3):326-33; Hatfield G W, Roth D A. Optimizing scaleup yield for protein production: Computationally Optimized DNA Assembly (CODA) and Translation Engineering. Biotechnol Annu Rev. 2007; 13:27-42; and/or Moura G R, Pinheiro M, Freitas A, Oliveira J L, Frommlet J C, Carreto L, Soares A R, Bezerra A R, Santos M A. Species-specific codon context rules unveil non-neutrality effects of synonymous mutations. PLoS One. 2011; 6(10):e26817). Moreover, hidden stop codon (HSC) count can be maximized to increase gene expression, since the presence of hidden stop codons (HSC) also prevents off-frame reading. Particularly preferred tools for codon-optimization include COOL (URL: http://cool.syncti.org/; Ju Xin Chin, Bevan Kai-Sheng Chung, Dong-Yup Lee; Codon Optimization OnLine (COOL): a web-based multi-objective optimization platform for synthetic gene design, Bioinformatics, Volume 30, Issue 15, 1 Aug. 2014, Pages 2210-2212); “OptimumGene™-Codon Optimization” (GenScript; as described in US 2011/0081708 A1) and the “Codon Optimization Tool” (IDT® Integrated DNA Technologies; URL: http://eu.idtdna.com/CodonOpt).

Alternatively, it is also preferred that the DNA molecule, or at least the nucleotide sequence of the DNA molecule, which encodes the (poly)peptide of interest, is not codon-optimized. In particular, it is preferred that the DNA molecule or said nucleotide sequence is not codon-optimized, if the (poly)peptide of interest is a human (poly)peptide or is derived from a human (poly)peptide.

Preferably, the DNA molecule, which comprises the nucleotide sequence encoding the (poly)peptide of interest, further comprises (in addition to the nucleotide sequence encoding the (poly)peptide of interest) an intronic sequence upstream and/or downstream of the nucleotide sequence encoding the (poly)peptide of interest. More preferably, the DNA molecule comprising the nucleotide sequence encoding the (poly)peptide of interest further comprises (in addition to the nucleotide sequence encoding the (poly)peptide of interest) (i) an intronic sequence upstream of the nucleotide sequence encoding the (poly)peptide of interest and (ii) an intronic sequence downstream of the nucleotide sequence encoding the (poly)peptide of interest. In particular, the term “intronic sequence” refers to a non-coding nucleotide sequence.

Preferably, the intronic sequence has a length of at least 5 nucleotides (in dsDNA: base pairs; bp), preferably at least 10 nucleotides (in dsDNA: base pairs; bp), more preferably at least 15 nucleotides (in dsDNA: base pairs; bp), even more preferably at least 20 nucleotides (in dsDNA: base pairs; bp) and most preferably at least 25 nucleotides (in dsDNA: base pairs; bp). It is also preferred that the intronic sequence has a length of at least 50 nucleotides (in dsDNA: base pairs; bp), preferably at least 75 or 100 nucleotides (in dsDNA: base pairs; bp), more preferably at least 150 or 200 nucleotides (in dsDNA: base pairs; bp), even more preferably at least 300 or 400 nucleotides (in dsDNA: base pairs; bp), still more preferably at least 500 nucleotides (in dsDNA: base pairs; bp) and most preferably at least 600 nucleotides (in dsDNA: base pairs; bp).

It is also preferred that the intronic sequence has a length of no more than 3000 nucleotides, preferably no more than 2500 nucleotides (in dsDNA: base pairs; bp), more preferably no more than 2000 nucleotides (in dsDNA: base pairs; bp), even more preferably (in particular if the DNA molecule comprises two intronic sequences) no more than 1500 nucleotides (in dsDNA: base pairs; bp), still more preferably (in particular if the DNA molecule comprises two intronic sequences) no more than 1250 nucleotides (in dsDNA: base pairs; bp), and most preferably (in particular if the DNA molecule comprises two intronic sequences) no more than 1000 nucleotides (in dsDNA: base pairs; bp).

Preferably, the intronic sequence comprises a splice recognition site. A “splice recognition site” (also referred to as “splice site”) is a nucleotide sequence, which is specifically recognized by the spliceosome and spliced. Accordingly, the splice (recognition) site is an intronic nucleotide sequence at the “border” between intron and exon.

More preferably, the DNA molecule comprising the nucleotide sequence encoding the (poly)peptide of interest comprises:
(i) an intronic sequence comprising a (single) splice recognition site upstream of the nucleotide sequence encoding a (poly)peptide of interest (e.g., a 5′ splice site); and
(ii) an intronic sequence comprising a (single) splice recognition site downstream of the nucleotide sequence encoding a (poly)peptide of interest (e.g., a 3′ splice site). Most preferably, the splice recognition site is located directly adjacent (directly upstream or downstream) of the nucleotide sequence encoding the (poly)peptide of interest.

As used herein, the term “5′ splice site” refers to a splice site, which is located at the downstream end of an intron and, therefore, directly upstream of an exon (at the 5′ end of the exon). For example, a “5′ splice site” typically comprises the nucleotides “AG” (in this order) at its 3′ end. In other words, a “5′ splice site” may end with the nucleotides “AG” as last two nucleotides (thereafter the exon/coding sequence starts).

As used herein, the term “3′ splice site” refers to a splice site, which is located at the upstream end of an intron and, therefore, directly downstream of an exon (at the 3′ end of the exon). For example, a “3′ splice site” typically comprises the nucleotides “GT” (in this order) at its 5′ end. In other words, a “3′ splice site” may start with the nucleotides “GT” as first two nucleotides (just (directly) after the end of the exon/coding sequence).

Splice sites can be predicted in silico by various bioinformatics tools, including, for example:
Berkeley Drosophila Genome Project (Drosophily and human prediction; URL: http://www.fruitfly.org/seq_tools/splice.html; Reese M G, Eeckman, F H, Kulp, D, Haussler, D, 1997. “Improved Splice Site Detection in Genie”. J Comp Biol 4(3), 311-23);
Human Splicing Finder (URL: http://www.umd.be/HSF/; FO Desmet, Hamroun D, Lalande M, Collod-Beroud G, Claustres M, Beroud C. Human Splicing Finder: an online bioinformatics tool to predict splicing signals. Nucleic Acid Research, 2009, April);
GeneSplicer (URL: http://ccb.jhu.edu/software/genesplicer/; M. Pertea, X. Lin, S. L. Salzberg. GeneSplicer: a new computational method for splice site prediction. Nucleic Acids Res. 2001 Mar. 1; 29(5):1185-90);
NetGene2 Server (URL: http://www.cbs.dtu.dk/services/NetGene2/; S. M. Hebsgaard, P. G. Korning, N. Tolstrup, J. Engelbrecht, P. Rouze, S. Brunak: Splice site prediction in Arabidopsis thaliana DNA by combining local and global sequence information, Nucleic Acids Research, 1996, Vol. 24, No. 17, 3439-3452. Brunak, S., Engelbrecht, J., and Knudsen, S.: Prediction of Human mRNA Donor and Acceptor Sites from the DNA Sequence, Journal of Molecular Biology, 1991, 220, 49-65);
SplicePort: An Interactive Splice Site Analysis Tool (URL: http://spliceport.cbcb.umd.edu/; Dogan R I, Getoor L, Wilbur W J, Mount S M. SplicePort—An interactive splice-site analysis tool. Nucleic Acids Research. 2007; 35(Web Server issue):W285-W291. doi:10.1093/nar/gkm407); and
MaxEntScan (URL: http://genes.mitedu/burgelab/maxent/Xmaxentscan_scoreseq.html; Yeo G, Burge C B. Maximum entropy modeling of short sequence motifs with applications to RNA splicing signals. J Comput Biol. 2004; 11(2-3):377-94.

Preferably, the 3′ splice site comprises a nucleotide sequence according to SEQ ID NO: 1 or a sequence variant thereof:

AGGTAAGT [SEQ ID NO: 1].

It is also preferred that the 5′ splice site comprises a polypyrimidine tract (10 U or C, followed by any base and C) and a terminal AG.

In a particularly preferred embodiment, the DNA molecule comprising the nucleotide sequence encoding the (poly)peptide of interest further comprises:
(i) upstream of the nucleotide sequence encoding a (poly)peptide of interest (at the 5′ end of the nucleotide sequence encoding a (poly)peptide of interest): an intronic sequence, in particular a 3′ end of a (naturally occurring) intron, comprising a (single) splice recognition site, in particular a 5′ splice site; and
(ii) downstream of the nucleotide sequence encoding a (poly)peptide of interest (at the 3′ end of the nucleotide sequence encoding a (poly)peptide of interest): an intronic sequence, in particular a 5′ end of an intron, comprising a (single) splice recognition site, in particular a 3′ splice site.

Most preferably, the DNA molecule comprising a nucleotide sequence encoding a (poly)peptide of interest comprises (in the following order):
1. a first intronic sequence comprising a first splice recognition site (for example, a 3′ end of a (naturally occurring) intron comprising the 5′ splice site);
2. the nucleotide sequence encoding the (poly)peptide of interest; and
3. a second intronic sequence comprising a second splice recognition site (for example, a 5′ end of a (naturally occurring) intron comprising the 3′ splice site).

A schematic example of such a DNA molecule (for example integrated in the switch region of chromosome 14 of the B lymphocyte's genome) is shown in FIG. 11, in particular in the upper part for the general concept (the middle and lower part of FIG. 11 show examples thereof including further features as described below).

A particularly preferred example of an intronic sequence comprises or consists of a nucleotide sequence according to any one of SEQ ID NOs 112-115 or a sequence variant thereof as defined herein. For example, an intronic sequence located (directly) upstream of the nucleotide sequence encoding the (poly)peptide of interest (at the 5′ end of the nucleotide sequence encoding the (poly)peptide of interest) preferably comprises or consists of a nucleotide sequence according to SEQ ID NO: 112 or SEQ ID NO: 114, or a sequence variant thereof as defined herein. For example, an intronic sequence located (directly) downstream of the nucleotide sequence encoding the (poly)peptide of interest (at the 3′ end of the nucleotide sequence encoding the (poly)peptide of interest) preferably comprises or consists of a nucleotide sequence according to SEQ ID NO: 113 or SEQ ID NO: 115, or a sequence variant thereof as defined herein.

Preferably, the intronic sequence contains an Ig-locus intronic sequence. The term “Ig-locus intronic sequence” refers to an intronic sequence of an immunoglobulin (Ig) locus (in particular an intronic sequence which naturally occurs in an Ig locus, such as a 5′ end and/or a 3′ end of an intron naturally occurring in an Ig locus). Accordingly, it is preferred that the intronic sequence is a fragment of a (naturally occurring) intron of an Ig locus or a complete intron of an Ig locus.

Most preferably, the intronic sequence comprises an intronic sequence of a J-segment downstream intron and/or an intronic sequence of a CH-upstream intron, for example a CH1-upstream intron. The term “J-segment downstream intron” refers to the intron directly downstream of the exon encoding the J segment. The term “CH upstream intron” refers to the intron directly upstream of an exon encoding a heavy chain constant domain (CH). Accordingly, an intronic sequence of a J-segment downstream intron and/or an intronic sequence of a CH-upstream intron may be a complete J-segment downstream intron/complete CH-upstream intron or a fragment thereof as described above. Preferably, the CH-upstream intron includes a branchpoint sequence (also known as “branch sequence” or “branch site”).

Particularly preferably, the DNA molecule comprises an intronic sequence of a CH-upstream intron (e.g., a CH1-upstream intron) upstream of the nucleotide sequence encoding the (poly)peptide of interest (i.e. the intronic sequence of CH-upstream intron is located (directly) “before”/upstream of the nucleotide sequence encoding the (poly)peptide of interest) and/or an intronic sequence of a J-segment downstream intron downstream of the nucleotide sequence encoding the (poly)peptide of interest (i.e. intronic sequence of the J-segment downstream intron is located (directly) “before”/downstream of the nucleotide sequence encoding the (poly)peptide of interest).

Most preferably, the DNA molecule comprising a nucleotide sequence encoding a (poly)peptide of interest comprises (in the following order):
1. an intronic sequence of a CH-upstream intron (for example a 3′ end fragment of a CH-upstream intron) comprising at its 3′ end a first splice recognition site (e.g., a 5′ slice site), the intronic sequence of the CH-upstream intron preferably further comprising a branchpoint sequence and/or an intronic splicing enhancer);
2. the nucleotide sequence encoding a (poly)peptide of interest;
3. an intronic sequence of a J-segment downstream intron (for example a 5′ end fragment of a J-segment downstream intron) comprising at its 5′ end a second splice recognition site (e.g., a 3′ slice site), the intronic sequence of the J-segment downstream intron preferably further comprising an intronic splicing enhancer).

Such a preferred embodiment is schematically shown in FIG. 11 (middle).

Accordingly, it is preferred that an intronic sequence upstream of the nucleotide sequence encoding a (poly)peptide of interest comprises a branchpoint sequence (also known as “branch sequence” or “branch site”). A “branch site” is a weakly conserved sequence element, such as YNCTGAC (wherein Y may be C or T and N may be any nucleotide selected from A, G, C and T; SEQ ID NO: 2), located at a conserved distance of about 18-50 nucleotides from the 5′ splice site. Accordingly, it is preferred that the branch site is located in the intronic sequence about 18-50 nucleotides (preferably 20-40 nucleotides) upstream of a 5′ splice site comprised in the intronic sequence.

The intronic sequence may also comprise a splicing regulatory element (SRE), which is a cis-acting sequence, which either enhances or silences (suppresses) splicing. Accordingly, “splicing enhancers” and “splicing silencers” can be distinguished. SREs recruit trans-acting splicing factors to activate or suppress the splice site recognition or spliceosome assembly.

Preferably, the intronic sequence comprises a (intronic) splicing enhancer. It is also preferred that the intronic sequence does not comprise a (intronic) splicing silencer. In general, splicing enhancers/silencers present in an intronic sequence (e.g. in (a fragment of) an intron) are referred to as “intronic” splicing enhancers/silencers, while splicing enhancers/silencers present in an exonic/coding sequence (e.g. in the nucleotide sequence encoding the (poly)peptide of interest) are referred to as “exonic” splicing enhancers/silencers. Presence of natural or designed splicing enhancers and/or absence of splicing silencers in the DNA molecule is preferred and may improve splicing and integration of the nucleotide sequence encoding the (poly)peptide of interest.

It is also preferred that the DNA molecule comprises an intronic sequence comprising an intronic splicing enhancer upstream and/or downstream of the nucleotide sequence encoding a (poly)peptide of interest. Preferred intronic splicing enhancers are described in Wang Y, Ma M, Xiao X, Wang Z. Intronic Splicing Enhancers, Cognate Splicing Factors and Context Dependent Regulation Rules. Nature structural &molecular biology. 2012; 19(10):1044-1052. doi:10.1038/nsmb.2377.

Most preferably, the intronic splicing enhancer has a nucleotide sequence according to any one of SEQ ID NOs 3-26, or a sequence variant thereof:

(SEQ ID NO: 3)
GTAGTGAGGG
(SEQ ID NO: 4)
GTTGGTGGTT
(SEQ ID NO: 5)
AGTTGTGGTT
(SEQ ID NO: 6)
GTATTGGGTC
(SEQ ID NO: 7)
AGTGTGAGGG
(SEQ ID NO: 8)
GGGTAATGGG
(SEQ ID NO: 9)
TCATTGGGGT
(SEQ ID NO: 10)
GGTGGGGGTC
(SEQ ID NO: 11)
GGTTTTGTTG
(SEQ ID NO: 12)
TATACTCCCG
(SEQ ID NO: 13)
GTATTCGATC
(SEQ ID NO: 14)
GGGGGTAGG
(SEQ ID NO: 15)
GTAGTTCCCT
(SEQ ID NO: 16)
GTTAATAGTA
(SEQ ID NO: 17)
TGCTGGTTAG
(SEQ ID NO: 18)
ATAGGTAACG
(SEQ ID NO: 19)
TCTGAATTGC
(SEQ ID NO: 20)
TCTGGGTTTG
(SEQ ID NO: 21)
CATTCTCTTT
(SEQ ID NO: 22)
GTATTGGTGT
(SEQ ID NO: 23)
GGAGGGTTT
(SEQ ID NO: 24)
TTTAGATTTG
(SEQ ID NO: 25)
ATAAGTACTG
(SEQ ID NO: 26)
TAGTCTATTA

Most preferably, the intronic sequence has a nucleotide sequence according to any one of SEQ ID NOs 27-53, or a sequence variant thereof. SEQ ID NOs 27-45 show preferred examples of (intronic sequences/fragements of) CH upstream introns and SEQ ID NOs 46-51 show preferred examples of (intronic sequences/fragments of) 1-segment downstream introns.

5′IgM-CH1
[SEQ ID NO: 27]
CGAGGAGGCAGCTCCTCACCCTCCCTTTCTCTTTGTCCTGCGGGTCCTCA
G
5′IgM-CH2
[SEQ ID NO: 28]
CGAAGGGGGCGGGAGTGGCGGGCACCGGGCTGACACGTGTCCCTCACTGC
AG
5′IgM-CH3
[SEQ ID NO: 29]
TCCGCCCACATCCACACCTGCCCCACCTCTGACTCCCTTCTCTTGACTCC
AG
5′IgM-CH4
[SEQ ID NO: 30]
CCACAGGCTGGTCCCCCCACTGCCCCGCCCTCACCACCATCTCTGTTCAC
AG
5′IgG1-CH1
[SEQ ID NO: 31]
TGGGCCCAGCTCTGTCCCACACCGCGGTCACATGGCACCACCTCTCTTGC
AG
5′ÌgG1-hinge
[SEQ ID NO: 32]
GGACACCTTCTCTCCTCCCAGATTCCAGTAACTCCCAATCTTCTCTCTGC
AG
5′IgG1-CH2
[SEQ ID NO: 33]
AGGGACAGGCCCCAGCCGGGTGCTGACACGTCCACCTCCATCTCTTCCTC
AG
5′IgG1-CH3
[SEQ ID NO: 34]
GGCCCACCCTCTGCCCTGAGAGTGACCGCTGTACCAACCTCTGTCCCTAC
AG
5′IgG3-CH1
[SEQ ID NO: 35]
TGGGCCCAGCTCTGTCCCACACCGCAGTCACATGGCGCCATCTCTCTTGC
AG
5′IgG3-hinge
[SEQ ID NO: 36]
AGATACCTICTCTCTTCCCAGATCTGAGTAACTCCCAATCTTCTCTCTGC
AG
5′IgG3-hinge2
[SEQ ID NO: 37]
ACGCATCCACCTCCATCCCAGATCCCCGTAACTCCCAATCTTCTCTCTGC
AG
5′IgG3-hinge3
[SEQ ID NO: 38]
ACGCGTCCACCTCCATCCCAGATCCCCGTAACTCCCAATCTTCTCTCTGC
AG
5′IgG3-hinge4
[SEQ ID NO: 39]
ACGCATCCACCTCCATCCCAGATCCCCGTAACTCCCAATCTTCTCTCTGC
AG
5′IgG3-CH2
[SEQ ID NO: 40]
ACGCATCCACCTCCATCCCAGATCCCCGTAACTCCCAATCTTCTCTCTGC
AG
5′IgG3-CH3
[SEQ ID NO: 41]
GACCCACCCTCTGCCCTGGGAGTGACCGCTGTGCCAACCTCTGTCCCTAC
AG
5′IgG4-CH1
[SEQ ID NO: 42]
TGGGCCCAGCTCTGTCCCACACCGCGGTCACATGGCACCACCTCTCTTGC
AG
5′IgG4-hinge
[SEQ ID NO: 43]
AGACACCTTCTCTCCTCCCAGATCTGAGTAACTCCCAATCTTCTCTCTGC
AG
5′IgG4-CH2
[SEQ ID NO: 44]
AGGGACAGGCCCCAGCCGGGTGCTGACGCATCCACCTCCATCTCTTCCTC
AG
5′IgG4-CH3
[SEQ ID NO: 45]
GGCCCACCCTCTGCCCTGGGAGTGACCGCTGTGCCAACCTCTGTCCCTAC
AG

The above examples show fragments (3′ ends) of naturally occurring introns in the respective Ig locus (e.g. IgM-CH1) of IgM and various IgG subclasses. It is also preferred that corresponding (naturally occurring) intronic sequences of other immunoglobulin isotypes such as IgA1, IgA2, and IgE, may be used.

3′J1
[SEQ ID NO: 46]
GTGAGTCTGCTGTCTGGGGATAGCGGGGAGCCAGGTGTACTGGGCCAGGC
AA
3′J2
[SEQ ID NO: 47]
GTGAGTCCCACTGCAGCCCCCTCCCAGTCTTCTCTGTCCAGGCACCAGGC
CA
3′J3
[SEQ ID NO: 48]
GTAAGATGGCTTTCCTTCTGCCTCCTTTCTCTGGGCCCAGCGTCCTCTGT
CC
3′J4
[SEQ ID NO: 49]
GTGAGTCCTCACAACCTCTCTCCTGCTTTAACTCTGAAGGGTTTTGCTGC
AT
3′J5
[SEQ ID NO: 50]
GTGAGTCCTCACCACCCCCTCTCTGAGTCCACTTAGGGAGACTCAGCTTG
CC
3′J6
[SEQ ID NO: 51]
GTAAGAATGGCCACTCTAGGGCCTTTGTTTTCTGCTACTGCCTGTGGGGT
TT
Further preferred examples include:
5′LAIR1
[SEQ ID NO: 52]
CATGGTGACTTCCTACAGTGGACGCTGAGATCCTGCTCTGCTTCCCTCCT
AG
3′LAIR1
[SEQ ID NO: 53]
GTGAGGACGTCACCTGGGCCCTGCCCCAGTCTCAGCTCGACCCTCGAGCT
TG

In general, it is preferred that the DNA molecule comprises a splicing enhancer. The splicing enhancer may be intronic (i.e., located in an intronic sequence of the DNA molecule) or exonic (i.e., located in a coding sequence of the DNA molecule, e.g. in the nucleotide sequence encoding the (poly)peptide of interest). As the nucleotide sequence encoding the (poly)peptide of interest is in general much more predetermined (due to its functionality of encoding the (poly)peptide of interest) than an intronic sequence, an intronic splicing enhancer is usually preferred. In other words, it is usually preferred that the splicing enhancer is located in an intronic sequence comprised in the DNA molecule. More preferably, the DNA molecule comprises an intronic sequence upstream of the nucleotide sequence encoding the (poly)peptide of interest and an intronic sequence downstream of the nucleotide sequence encoding the (poly)peptide of interest, and each of the intronic sequences comprises a splicing enhancer. Such a preferred embodiment is schematically shown in FIG. 11 (lower part).

However, it is also preferred that the DNA molecule comprises an exonic splicing enhancer, for example in the nucleotide sequence encoding the (poly)peptide of interest. For example, the (poly)peptide of interest may be selected such that the nucleotide sequence encoding it “naturally” comprises an exonic splicing enhancer. Moreover, the degeneracy of the genetic code may be used to introduce an exonic splicing enhancer. That is, silent mutations (which do not change the encoded amino acid) may be used to introduce an exonic splicing enhancer into the nucleotide sequence encoding the (poly)peptide of interest.

Preferably, the DNA molecule does not comprise an exonic splicing silencer.

Exonic splicing enhancers (ESEs) are discrete sequences within exons, that promote both constitutive and regulated splicing. Exonic splicing enhancer (ESE) sequences are bound by serine & arginine-rich (SR) proteins, which in turn enhance the recruitment of splicing factors. Preferably, an exonic splicing enhancer is a sequence motif consisting of six bases.

Exonic splicing enhancers are known in the art (Liu H-X, Zhang M, Krainer A R. Identification of functional exonic splicing enhancer motifs recognized by individual SR proteins. Genes & Development. 1998; 12(13)1998-2012; Blencowe B J. Exonic splicing enhancers: mechanism of action, diversity and role in human genetic diseases. Trends Biochem Sci. 2000 March; 25(3):106-10). Splicing enhancers can be predicted in silico by various bioinformatics tools, including, for example by RESCUE-ESE Web Server (URL: http://genes.mit.edu/burgelab/rescue-ese/; Fairbrother W G, Yeh R F, Sharp P A, Burge C B. Predictive identification of exonic splicing enhancers in human genes. Science. 2002 Aug. 9; 297(5583):1007-13) and/or by ESEfinder (URL: http://rulai.cshLedu/cgi-bin/tools/ESE3/esefinder.cgi?process=home; Smith, P. J., Zhang, C., Wang, J. Chew, S. L., Zhang, M. Q. and Krainer, A. R. 2006. An increased specificity score matrix for the prediction of SF2/ASF-specific exonic splicing enhancers. Hum. Mol. Genet. 15(16): 2490-2508; Cartegni L., Wang J., Zhu Z., Zhang M. Q., Krainer A. R.; 2003. ESEfinder: a web resource to identify exonic splicing enhancers. Nucleic Acid Research, 2003, 31(13): 3568-3571).

Most preferably, the splicing enhancer (intronic or exonic) is selected to show efficiency in isolated human B cells, in particular in primary human B cells. Accordingly, the splicing enhancer is preferably derived from an isolated human B cells, in particular from a primary human B cell (e.g., by analyzing B cell sequences with appropriate bioinformatics tools for prediction of splicing enhancers as described herein.

Preferably, in the method according to the present invention the genome of the B lymphocyte is edited to express a modified immunoglobulin chain comprising in N- to C-terminal direction: a variable domain, the (poly)peptide of interest (encoded by the DNA molecule introduced in step (ii) of the method according to the present invention) and a constant domain. In other words, the genome of the B lymphocyte is preferably edited to express a modified immunoglobulin chain comprising the (poly)peptide of interest arranged between a variable domain and a constant domain of the immunoglobulin chain. Accordingly, it is preferred that the genome of the B lymphocyte is edited to express a modified antibody comprising the (poly)peptide of interest in the elbow region of the antibody. Moreover, it is preferred that the genome of the B lymphocyte is edited to express a modified B-cell receptor comprising the (poly)peptide of interest in the elbow region of the antibody.

The elbow region is the junction between the variable domains and the constant domains in the heavy and light chains of the antibody. Typically, the C-terminus of the variable domain (VH or VL) is directly linked to the N-terminus of the most N-terminal constant domain (typically CH1 or CL), and the junction between the C-terminus of the variable domain (VH or VL) and the N-terminus of the most N-terminal constant domain (typically CH1 or CL) is referred to as “elbow” or “elbow region”. The elbow region allows for bending and rotation of the variable domains relative to the constant domains. Accordingly, together with the hinge region, the elbow region provides flexibility to the antibody for antigen binding. The elbow region is also referred to as “molecular ball-and-socket joint” based on the range of motion provided by the elbow region (Lesk A M, Chothia C. Elbow motion in the immunoglobulins involves a molecular ball-and-socket joint. Nature. 1988 Sep. 8; 335(6186)188-90).

In the genome of B lymphocytes the switch region is located between the variable domain and the (most N-terminal) constant domain of an antibody. As described above, by action of AID, the DNA molecule comprising a nucleotide sequence encoding the (poly)peptide of interest is integrated in the switch region of the B cell genome. Accordingly, the DNA molecule comprising a nucleotide sequence encoding the (poly)peptide of interest is integrated between the variable domains and the constant domains of an antibody. In particular, intronic sequences are removed during splicing, such that an immunoglobulin chain expressed by a B cell genome edited according to the present invention as described herein comprises in N- to C-terminal direction: a variable domain, the (poly)peptide of interest (encoded by the DNA molecule introduced in step (ii) of the method according to the present invention) and a constant domain. Accordingly, in the expressed immunoglobulin chain the (poly)peptide of interest is located in the elbow region of the antibody, i.e. between variable and the most N-terminal constant domain.

Preferred examples of antibodies comprising a (poly)peptide of interest in the elbow region and the corresponding genomic arrangements are shown in FIG. 2A. The upper part of FIG. 2A shows an antibody (of classical IgG-type) with a single receptor domain as (poly)peptide of interest in the elbow region. The middle part of FIG. 2A shows an antibody (of classical IgG-type) with two receptor domains as (poly)peptide of interest in the elbow region. The lower part of FIG. 2A shows an antibody (of classical IgG-type) with a V_{H} domain and a V_{L} domain as (poly)peptide of interest in the elbow region.

In general, antibodies with “In-Elbow-Inserts” (IEI), which can be obtained with the method according to the present invention as described herein, are described in detail in WO 2019/025391 A1 and in WO 2019/024979 A1 (PCT/EP2017/069357), which are incorporated herein by reference. In particular, the genome of a B lymphocyte may be edited with the method according to the present invention to express an antibody, or an antigen binding fragment thereof, comprising a heavy chain, wherein said heavy chain comprises in N- to C-terminal direction
(i) a variable domains, in particular a heavy chain variable domain (VH);
(ii) the (poly)peptide of interest; and
(iii) one or more constant domains, in particular heavy chain constant domains (CH), preferably comprising at least a CH1 constant domain.

Preferably, the (poly)peptide of interest (ii) of the heavy chain does preferably not comprise a fragment of the light chain.

Such antibodies engineered in the elbow region to contain a (poly)peptide of interest can, for example, simultaneously bind (1) to the antigen targeted by their variable domains and (2) to an additional target targeted by a binding site introduced into the antibody's elbow region—as described in detail in WO 2019/025391 A1 and in WO 2019/024979 A1 (PCT/EP2017/069357).

In particular, a variety of “In-Elbow-Inserts” (IEI) antibodies may be obtained with the method according to the present invention as described herein. “In-Elbow-Inserts” (IEI) antibodies are described in detail in WO 2019/025391 A1 and in WO 2019/024979 A1 (PCT/EP2017/069357). Preferred “In-Elbow-Inserts” (IEI) antibodies, which are obtainable with the method according to the present invention correspond to preferred embodiments of the “In-Elbow-Inserts” (IEI) antibodies described in WO 2019/025391 A1 and in WO 2019/024979 A1 (PCT/EP2017/069357).

However, with the method according to the present invention also other recombinant antibodies can be obtained. In particular, it is also preferred that the genome of the B lymphocyte is edited to express a modified immunoglobulin chain, wherein an endogenous variable domain is replaced by the (poly)peptide of interest. Accordingly, it is also preferred that the genome of the B lymphocyte is edited to express a modified B-cell receptor, wherein an endogenous variable domain is replaced by the (poly)peptide of interest. Accordingly, it is preferred that the genome of the B lymphocyte is edited to express a modified antibody comprising the (poly)peptide of interest “instead” of an endogenous variable domain.

Preferred examples of antibodies comprising immunoglobulin chains comprising a (poly)peptide of interest instead of the endogenous variable region and the corresponding genomic arrangements are shown in FIG. 2B. The upper part of FIG. 2B shows an antibody (of classical igG-type), wherein the endogenous variable region (V_{H}) was replaced by another (heterologous) variable region (V_{H}). In the next construct, the endogenous variable region (V_{H}) was replaced by a receptor domain and another (heterologous) variable region (V_{H}). In the next construct, the endogenous variable region (Vᵤ) was replaced by three (heterologous) variable regions (V_{H}-V_{L}-V_{H}). In the lowest part of FIG. 2B the endogenous variable region (V_{H}) was replaced by two (heterologous) variable regions and a (heterologous) constant region (V_{L}-C_{L}-V_{H}). The term “heterologous” refers to a sequence, which is distinct from the endogenous sequence, i.e. the sequence, which was originally at this genomic location.

It is also preferred that the genome of the B lymphocyte is edited to express a modified immunoglobulin chain, wherein the endogenous constant domains are replaced by the (poly)peptide of interest. Accordingly, it is also preferred that the genome of the B lymphocyte is edited to express a modified B-cell receptor, wherein the endogenous constant domains are replaced by the (poly)peptide of interest. Accordingly, it is preferred that the genome of the B lymphocyte is edited to express a modified antibody comprising the (poly)peptide of interest “instead” of the endogenous constant domains. Accordingly, such a modified immunoglobulin chain comprises an (endogenous) variable domain, the (poly)peptide of interest, but no (endogenous) constant domain.

Modifications, wherein the (poly)peptide of interest replaces the endogenous variable domain may be achieved by introduction of a nucleotide sequence encoding a cleavage site, such as a T2A cleavage site, between the endogenous VDJ exon and the nucleotide sequence encoding the (poly)peptide of interest. Modifications, wherein the (poly)peptide of interest replaces the endogenous constant domains may be achieved by introduction of a nucleotide sequence encoding a cleavage site, such as a T2A cleavage site, between the nucleotide sequence encoding the (poly)peptide of interest and the nucleotide sequence encoding the constant regions.

Accordingly, it is preferred that the DNA molecule comprises a nucleotide sequence encoding a cleavage site upstream and/or downstream of the nucleotide sequence encoding a (poly)peptide of interest. Preferably, the cleavage site is a T2A cleavage site.

As used herein the term “cleavage site” includes enzymatic cleavage (e.g. by proteases) as well as to “self-cleavage”, e.g. by ribosomal skipping. Sites for enzymatic cleavage are known in the art. Preferred examples include the 3C (“PreScission”) cleavage tag for human rhinovirus (HRV) 3C protease (Sequence: LEVLFQGP; SEQ ID NO: 54); EKT (Enterokinase) cleavage tag for Enterokinase (Sequence: DDDDK; SEQ ID NO: 55); FXa (Factor Xa) cleavage tag for Factor Xa (Sequence: IEGR; SEQ ID NO: 56); TEV (tobacco etch virus) cleavage tag for Tobacco etch virus protease (Sequence: ENLYFQG; SEQ ID NO: 57); and Thrombin cleavage tag for thrombin (Sequence: LVPRGS; SEQ ID NO: 58). In general, sites for cleavage by proteases or peptidases allow to—posttranslationally—cleave the protein translated from the modified immunoglobulin gene. By such a protein cleavage, e.g. by a peptidase or proteinase, the covalently linked immunoglobulin components comprised in the translated gene product (one single chain) are processed into fragments, thereby achieving the modified antibodies or antibody fragments as described above.

Moreover, cleavage sites can be predicted in silico by various bioinformatics tools, including, for example:
PeptideCutter (URL: https://web.expasy.org/peptide_cutter/; Gasteiger E., Hoogland C., Gattiker A., Duvaud S., Wilkins M. R., Appel R. D., Bairoch A.; Protein Identification and Analysis Tools on the ExPASy Server; (In) John M. Walker (ed): The Proteomics Protocols Handbook, Humana Press (2005)); PROSPER (URL: https://prospererc.monash.edu.au/webserver.html; Song J, Tan H,
Perry A J, Akutsu T, Webb G I, Whisstock J C and Pike R N. PROSPER: an integrated feature-based tool for predicting protease substrate cleavage sites. PLoS ONE, 2012, 7(11): e50300);
MEROPS (URL: https://www.ebi.ac.uk/merops/; Rawlings, N. D., Barrett, A. J., Thomas, P. D., Huang, X., Bateman, A. & Finn, R. D. (2018) The MEROPS database of proteolytic enzymes, their substrates and inhibitors in 2017 and a comparison with peptidases in the PANTHER database. Nucleic Acids Res 46, D624-D632);
TopFIND (URL: http://clipserve.clip.ubc.ca/topfind; Nikolaus Fortelny, Sharon Yang, Paul Pavlidis, Philipp F. Lange*, Christopher M. Overall*, Nucleic Acids Research 43 (D1), D290-D297 (2014)); and
CutDB (URL: http://cutdb.burnham.org/; Igarashi Y, Eroshkin A, Gramatikova S, Gramatikoff K, Zhang Y, Smith J W, Osterman A L, Godzik A. CutDB: a proteolytic event database. Nucleic Acids Res. 2007 January; 35(Database issue):D546-9).

Preferably, the cleavage site is a “self-cleavage” site (also referred to as “self-processing” site), such as a ribosomal skipping site. As used herein, the term “self-cleavage” (“self-processing”) relates to “cleavage” without proteases, for example by ribosomal skipping. Preferably, a nucleotide sequence encoding a self-processing site is a nucleotide sequence encoding the amino acid sequence Asp-Val/Ile-Glu-X-Asn-Pro-Gly-Pro, wherein X may be any amino acid (DX₁EX₂NPGP, wherein X₁ is Val or Ile and X₂ may be any (naturally occurring) amino acid; SEQ ID NO: 59). Ribosomal skipping leads to the provision of separate entities in the course of mRNA translation. The underlying mechanism is based on non-formation of a covalent linkage between two amino acids, i.e. G (Gly) and P (Pro) during mRNA translation. Accordingly, the mRNA translation is not interrupted by the non-formation of a covalent bond between the Gly/Pro, but rather proceeds without stopping the ribosomal activity on the mRNA. In particular, the ribosomes do not form a peptide bond between these amino acids, if a sequence pattern Asp-Val/Ile-Glu-X-Asn-Pro-Gly≠Pro occurs in a peptide sequence. Non-formation of a covalent bond occurs between the C-terminal Gly-Pro position of the above amino acid stretch. Preferred self-processing sites are 2A-sites, such as T2A (Sequence: EGRGSLLTCGDVEENPGP; SEQ ID NO: 60); F2A (Sequence: VKQTLNFDLLKLAGDVESNPGP; SEQ ID NO: 61); or P2A Sequence: ATNFSLLKQAGDVEENPGP; SEQ ID NO: 62); or sequence variants thereof as described herein, in particular the sequence variants according to SEQ ID NO: 63 (GSGATNFSLLKQAGDVEENPGP) or SEQ ID NO: 64 (RKRRGSGATNFSLLKQAGDVEENPGP).

Most preferably, the DNA molecule comprises a nucleotide sequence encoding a T2A site (for example SEQ ID NO: 60) or a sequence variant thereof as described herein.

In some embodiments, the DNA molecule does not comprise a full length-DNA strand of a chromosome.

In some embodiments, the DNA molecule comprises a promoter. More specifically, in some embodiments, the DNA molecule may comprise a transcription unit. The term “transcription unit” refers to a sequence of nucleotides in DNA that codes for a single RNA molecule, along with the sequences necessary for its transcription. Typically, a transcription unit comprises a promoter, an RNA-coding sequence, and a terminator. Examples of promoters and terminators are well-known in the art. For example, the promoter and the terminator may be the same as in the naturally occurring gene regarding the encoded (poly)peptide. In some embodiments, the promoter (and/or the terminator) is heterologous (with regard to the encoded (poly)peptide; i.e. it does not occur in the gene of the encoded (poly)peptide in nature). In particular, the RNA-coding sequence (and the corresponding DNA sequence in the DNA molecule) typically encodes a (poly)peptide of interest, for example as described herein.

(Poly)Peptide of Interest

The (poly)peptide of interest may be heterologous (i.e. not expressed by the B lymphocyte in nature) and/or it may be included in a polypeptide (or protein), which is heterologous (i.e. not expressed by the B lymphocyte in nature; such as a modified antibody).

As described above, a (poly)peptide of interest may be any (poly)peptide, in particular which is envisaged to be expressed as a (part of) a customized antibody or antibody fragment. In particular, the (poly)peptide of interest comprises or consists of one (single) or more functional domains. In general, the term “functional domain” refers to a functional unit, e.g. of the antibody or the antibody fragment. Typically, a functional domain provides the protein, e.g. the antibody or the antibody fragment, with an (additional) functionality. Accordingly, the (additional) functional domain usually contains all amino acids/sequences required to provide the (additional) function.

Preferably, the functional domain (comprised in the (poly)peptide of interest) has a length of up to 1000 amino acids, more preferably of up to 750 amino acids, even more preferably of up to 500 amino acids, still more preferably of up to 400 amino acids, particularly preferably of up to 300 amino acids and most preferably of up to 275 or 250 amino acids. Moreover, it is preferred that the functional domain has a length of 5 to 1000 amino acids, more preferably of 10 to 750 amino acids, even more preferably of 20 to 500 amino acids, still more preferably of 50 to 400 amino acids, particularly preferably of 70 to 300 amino acids and most preferably of 75 to 275 or of 100 to 250 amino acids.

It is also preferred that the functional domain (comprised in the (poly)peptide of interest) has a size of up to 150 kDa, more preferably of up to 100 kDa, even more preferably of up to 80 kDa, still more preferably of up to 70 kDa, particularly preferably of up to 50 kDa and most preferably of up to 30 or 25 kDa. Moreover, it is preferred that the functional domain has a size of 0.5 kDa to 150 kDa, more preferably of 1 kDa to 100 kDa, even more preferably of 2.5 kDa to 80 kDa, still more preferably of 5 kDa to 70 kDa, particularly preferably of 7.5 kDa to 50 kDa and most preferably of 10 kDa to 30 or 25 kDa.

The (poly)peptide of interest may comprise a monomeric domain or multimeric domains. A monomeric domain is a domain, which mediates its functionality without the involvement of any further (additional) domain. Multimeric domains, for example two domains forming a dimer or three domains forming a trimer, mediate their functionality together, in particular as multimer, e.g., as dimer or trimer. In case of multimeric domains, the (poly)peptide of interest may comprise linkers as described herein to provide sufficient flexibility to form the multimer, in particular a linker may be located (directly) adjacent to one or more multimeric domain(s), e.g. in between two multimeric domains or on each side of all multimeric domains. Preferably, the (poly)peptide of interest comprises or consists of one or more monomeric domain(s).

In general, the (poly)peptide of interest may comprise or consist of one single protein domain or of more than one protein domain. “More than one protein domain” may be multimeric domains as described above and/or one or more monomeric domains as described above. For example, the (poly)peptide of interest may comprise or consist of two or three monomeric domains, which may mediate the same or distinct functionality and/or which may optionally be connected by a linker. For example, the(poly)peptide of interest may comprise 1, 2, 3, 4, 5, 6, 7, 8, 9, or 10 (distinct) protein domains.

Preferably, the functional domain comprised in the (poly)peptide of interest (more preferably the complete (poly)peptide of interest), is a human protein, peptide or polypeptide or a fragment (in particular a domain) or derivative thereof.

The (poly)peptide of interest may also comprise a linker, for example a GS-linker.

A preferred functional domain (comprised in the (poly)peptide of interest) comprises or consists of an Ig-like domain; for example an Ig-like domain of a protein or (poly)peptide, e.g., as exemplified below. The basic structure of immunoglobulin (Ig) molecules is a tetramer of two light chains and two heavy chains linked by disulphide bonds. There are two types of light chains: kappa and lambda, each composed of a constant domain (CL) and a variable domain (VL). There are five types of heavy chains: alpha, delta, epsilon, gamma and mu, all consisting of a variable domain (VH) and three (in alpha, delta and gamma) or four (in epsilon and mu) constant domains (CH1 to CH4). Ig molecules are highly modular proteins, in which the variable and constant domains have clear, conserved sequence patterns. The domains in Ig and Ig-like molecules are grouped into four types: V-set (variable), C1-set (constant-1), C2-set (constant-2) and I-set (intermediate). Structural studies have shown that these domains share a common core Greek-key beta-sandwich structure, with the types differing in the number of strands in the beta-sheets as well as in their sequence patterns. Immunoglobulin-like domains that are related in both sequence and structure can be found in several diverse protein families. Ig-like domains are involved in a variety of functions, including cell-cell recognition, cell-surface receptors, muscle structure and the immune system.

Preferred examples of Ig-like domains include the Ig-like domains of any one or the following proteins or (poly)peptides: A1BG (alpha-1-B glycoprotein), ACAM, ADAMTSL1 (ADAMTS like 1), ADAMTSL3 (ADAMTS like 3), AGER (advanced glycosylation end-product specific receptor), ALCAM (activated leukocyte cell adhesion molecule), ALPK3 (alpha kinase 3), AMIGO1 (adhesion molecule with Ig like domain 1), AMIGO2 (adhesion molecule with Ig like domain 2), AMIGO3 (adhesion molecule with Ig like domain 3), AXL (AXL receptor tyrosine kinase), BCAM (basal cell adhesion molecule (Lutheran blood group)), BOC (BOC cell adhesion associated, oncogene regulated), BSG (basigin (Ok blood group)), BTLA (B and T lymphocyte associated), C10orf72, C20orf102, CADM1 (cell adhesion molecule 1), CADM3 (cell adhesion molecule 3), CADM4 (cell adhesion molecule 4), CCDC141 (coiled-coil domain containing 141), CD2, CD3, CD4, CD8, CD19, CD22, CD33, CD47, CD48, CD80, CD84, CD86, CD96, CD101, CD160, CD200, CD244, CD276, CDON (cell adhesion associated, oncogene regulated), CEACAM1 (carcinoembryonic antigen related cell adhesion molecule 1), CEACAM5 (carcinoembryonic antigen related cell adhesion molecule 5), CEACAM6 (carcinoembryonic antigen related cell adhesion molecule 6), CEACAM7 (carcinoembryonic antigen related cell adhesion molecule 7), CEACAM8 (carcinoembryonic antigen related cell adhesion molecule 8), CEACAM16 (carcinoembryonic antigen related cell adhesion molecule 16), CEACAM18 (carcinoembryonic antigen related cell adhesion molecule 18), CEACAM20 (carcinoembryonic antigen related cell adhesion molecule 20), CEACAM21 (carcinoembryonic antigen related cell adhesion molecule 21), CRL1 (cell adhesion molecule L1 like), CILP (cartilage intermediate layer protein), CILP2 (cartilage intermediate layer protein 2), CLMP (CXADR like membrane protein), CNTFR (ciliary neurotrophic factor receptor), CNTN1 (contactin 1), CNTN2 (contactin 2), CNTN3 (contactin 3, CNTN4 (contactin 4), CNTN5 (contactin 5), CNTN6 (contactin 6), CSF1R (colony stimulating factor 1 receptor), CXADR (CXADR, Ig-like cell adhesion molecule), DSCAM (DS cell adhesion molecule), DSCAML1 (DS cell adhesion molecule like 1), EMB (embigin), ESAM (endothelial cell adhesion molecule), F11R (F11 receptor), FAIM3, FCMR (Fc fragment of IgM receptor), HMCN1 (hemicentin 1), HMCN2 (hemicentin 2), FCAR (Fc fragment of IgA receptor), FCER1A (Fc fragment of IgE receptor Ia), FCGR1A (Fc fragment of IgG receptor Ia), FCGR1B (Fc fragment of IgG receptor Ib), FCGR1CP (Fc fragment of IgG receptor Ic, pseudogene), FCGR2A (Fc fragment of IgG receptor IIa), FCGR2B (Fc fragment of IgG receptor IIb), FCGR2C (Fc fragment of IgG receptor IIc), FCGR3A (Fc fragment of IgG receptor IIIa), FCGR3B (Fc fragment of IgG receptor IIIb), FCRH1, FCRH3, FCRH4, FCRL1 (Fc receptor like 1), FCRL2 (Fc receptor like 2), FCRL3 (Fc receptor like 3), FCRL4 (Fc receptor like 4), FCRL5 (Fc receptor like 5), FCRL6 (Fc receptor like 6), FCRLA (Fc receptor like A), FCRLB (Fc receptor like B), FGFR1, FGFR2, FGFR3, FGFR4, FGFRL1, FLT1 (fms related tyrosine kinase 1), FLT3 (fms related tyrosine kinase 3), FLT4 (fms related tyrosine kinase 4), FSTL4 (follistatin like 4), FSTL5 (follistatin like 5), GP6 (glycoprotein VI platelet), GPA33 (glycoprotein A33, GPR116, GPR125, ADGRF5 (adhesion G protein-coupled receptor F5), ADGRA2 (adhesion G protein-coupled receptor A2), hEMMPRIN, HEPACAM (hepatic and glial cell adhesion molecule), HEPACAM2 (HEPACAM family member 2), HLA-DMA, HLA-DMB, HLA-DQB, HLA-DQB1, HNT, HSPG2 (heparan sulfate proteoglycan 2), HYST2477, ICAM1 (intercellular adhesion molecule 1), ICAM2 (intercellular adhesion molecule 2), ICAM3 (intercellular adhesion molecule 3), ICAM4 (intercellular adhesion molecule 4 (Landsteiner-Wiener blood group)), ICAM5 (intercellular adhesion molecule 5), DCC (DCC netrin 1 receptor), NEO1 (neogenin 1), IGHA1, IGHD, IGHE, IGDCC4 (immunoglobulin superfamily DCC subclass member 4), IGLON5 (IgLON family member 5), IGSF1 (immunoglobulin superfamily member 1), IGSF2 (immunoglobulin superfamily member 2), IGSF3 (immunoglobulin superfamily member 3), IGSF5 (immunoglobulin superfamily member 5), IGSF9 (immunoglobulin superfamily member 9), IGSF9B (immunoglobulin superfamily member 9B), IGSF10 (immunoglobulin superfamily member 10), IGSF11 (immunoglobulin superfamily member 11), IGSF21 (immunoglobin superfamily member 21), IGSF23 (immunoglobulin superfamily member 23), IL1R1 (interleukin 1 receptor type 1), IL1R2 (interleukin 1 receptor type 2), IL1RAP (interleukin 1 receptor accessory protein), IL1RAPL1 (interleukin 1 receptor accessory protein like 1), IL1RAPL2 (interleukin 1 receptor accessory protein like 2), IL1RL1 (interleukin 1 receptor like 1), IL1RL2 (interleukin 1 receptor like 2), IL6R (interleukin 6 receptor), IL11RA (interleukin 11 receptor subunit alpha), IL12B (interleukin 12B), IL18BP (interleukin 18 binding protein), IL18R1 (interleukin 18 receptor 1), IL18RAP (interleukin 18 receptor accessory protein), ISLR2 (immunoglobulin superfamily containing leucine rich repeat 2), JAM2 (junctional adhesion molecule 2), JAM3 (junctional adhesion molecule 3), KDR (kinase insert domain receptor), KIR-123FM, KIR2DL1 (killer cell immunoglobulin like receptor, two Ig domains and long cytoplasmic tail 1), KIR2DL2 (killer cell immunoglobulin like receptor, two Ig domains and long cytoplasmic tail 2), KIR2DL3 (killer cell immunoglobulin like receptor, two Ig domains and long cytoplasmic tail 3), KIR2DL4 (killer cell immunoglobulin like receptor, two Ig domains and long cytoplasmic tail 4), KIR2DL5A (killer cell immunoglobulin like receptor, two Ig domains and long cytoplasmic tail 5A), KIR2DL5B (killer cell immunoglobulin like receptor, two Ig domains and long cytoplasmic tail 5B), KIR2DLX, KIR2DS1 (killer cell immunoglobulin like receptor, two Ig domains and short cytoplasmic tail 1), KIR2DS2 (killer cell immunoglobulin like receptor, two Ig domains and short cytoplasmic tail 2), KIR2DS3 (killer cell immunoglobulin like receptor, two Ig domains and short cytoplasmic tail 3), KIR2DS4 (killer cell immunoglobulin like receptor, two Ig domains and short cytoplasmic tail 4), KIR2DS5 (killer cell immunoglobulin like receptor, two Ig domains and short cytoplasmic tail 5), kir3d, K1R3DL1 (killer cell immunoglobulin like receptor, three Ig domains and long cytoplasmic tail 1), KIR3DL2 (killer cell immunoglobulin like receptor, three Ig domains and long cytoplasmic tail 2), KIR3DL3 (killer cell immunoglobulin like receptor, three Ig domains and long cytoplasmic tail 3), KIR3DP1 (killer cell immunoglobulin like receptor, three Ig domains pseudogene 1, KIR3DS1 (killer cell immunoglobulin like receptor, three Ig domains and short cytoplasmic tail 1), KIR3DX1 (killer cell immunoglobulin like receptor, three Ig domains X1), KIRREL1 (kirre like nephrin family adhesion molecule 1), KIRREL2 (kirre like nephrin family adhesion molecule 2), KIRREL3 (kirre like nephrin family adhesion molecule 3), KIT (KIT proto-oncogene receptor tyrosine kinase), L1 CAM, LAG3 (lymphocyte activating 3), LAIR1 (leukocyte associated immunoglobulin like receptor 1), LAIR2 (leukocyte associated immunoglobulin like receptor 2), LEPR (leptin receptor), LILRA1 (leukocyte immunoglobulin like receptor A1), LILRA2 (leukocyte immunoglobulin like receptor A2), LILRA3 (leukocyte immunoglobulin like receptor A3, LILRA4 (leukocyte immunoglobulin like receptor A4), LILRA5 (leukocyte immunoglobulin like receptor A5), LILRA6 (leukocyte immunoglobulin like receptor A6), LILRB1 (leukocyte immunoglobulin like receptor B1), LILRB2 (leukocyte immunoglobulin like receptor B2), LILRB3 (leukocyte immunoglobulin like receptor B3), LILRB4 (leukocyte immunoglobulin like receptor B4), LILRB5 (leukocyte immunoglobulin like receptor B5), LILRP2, LRIG1, LRIG2, LRIG3, LRIT1, LRRC4, LSAMP, LSR (lipolysis stimulated lipoprotein receptor), LY9 (lymphocyte antigen 9), MADCAM1 (mucosal vascular addressin cell adhesion molecule 1), MAG (myelin associated glycoprotein), MALT1 (MALT1 paracaspase), MCAM (melanoma cell adhesion molecule), MDGA1 (MAM domain containing glycosylphosphatidylinositol anchor 1), MDGA2 (MAM domain containing glycosylphosphatidylinositol anchor 2), MERTK (MER proto-oncogene, tyrosine kinase), MFAP3, MIR, MILR1 (mast cell immunoglobulin like receptor 1), MMP23A (matrix metallopeptidase 23A (pseudogene)), MMP23B (matrix metallopeptidase 23B), MUSK (muscle associated receptor tyrosine kinase), MXRAS (matrix remodeling associated 5), MYBPC3, MYOM1 (myomesin 1), MYOM2 (myomesin 2), MYOM3 (myomesin 3), NCA, NCAM1, NCAM2, NCR1 (natural cytotoxicity triggering receptor 1), NEGR1, NEO1, NFASC, NOPE, NPHS1 (NPHS1, nephrin), NPTN (neuroplastin), NRCAM (neuronal cell adhesion molecule), NTRK1 (neurotrophic receptor tyrosine kinase 1), NRG1, NT, NTRK3, OBSCN, OBSL1 (obscurin like 1), OPCML, OSCAR (osteoclast associated, immunoglobulin-like receptor), PAPLN, PDCD1 LG2 (programmed cell death 1 ligand 2), PDGFRA (platelet derived growth factor receptor alpha), PDGFRB (platelet derived growth factor receptor beta), PDGFRL (platelet derived growth factor receptor like), PECAM1 (platelet and endothelial cell adhesion molecule 1), PRODH2, PSG1 (pregnancy specific beta-1-glycoprotein 1), PSG2 (pregnancy specific beta-1-glycoprotein 2), PSG3 (pregnancy specific beta-1-glycoprotein 3), PSG4 (pregnancy specific beta-1-glycoprotein 4), PSG5 (pregnancy specific beta-1-glycoprotein 5), PSG6 (pregnancy specific beta-1-glycoprotein 6), PSG7 (pregnancy specific beta-1-glycoprotein 7 (gene/pseudogene)), PSG8 (pregnancy specific beta-1-glycoprotein 8), PSG9 (pregnancy specific beta-1-glycoprotein 9), PSG10 (pregnancy specific beta-1-glycoprotein 10), PSG11 (pregnancy specific beta-1-glycoprotein 11), PSG11s' (pregnancy specific beta-1-glycoprotein 11s'), PTGFRN (prostaglandin F2 receptor inhibitor), PTK7 (protein tyrosine kinase 7 (inactive)), PTPRD (protein tyrosine phosphatase, receptor type D), PTPRK (protein tyrosine phosphatase, receptor type K), PTPRM (protein tyrosine phosphatase, receptor type M), PTPRS protein tyrosine phosphatase, receptor type S), PTPRT (protein tyrosine phosphatase, receptor type T), PTPsigma, PUNC, PVR (poliovirus receptor), PVRL1, PVRL2, PVRL4, NECTIN1 (nectin cell adhesion molecule 1), NECTIN2 (nectin cell adhesion molecule 2), NECTIN3 (nectin cell adhesion molecule 3), RAGE, ROBO3 (roundabout guidance receptor 3), SCN1B (sodium voltage-gated channel beta subunit 1), SDK1 (sidekick cell adhesion molecule 1), SDK2 (sidekick cell adhesion molecule 2), SEMA3A (semaphorin 3A), SEMA3B (semaphorin 3B), SEMA3E (semaphorin 3E), SEMA3F (semaphorin 3F), SEMA3G (semaphorin 3G), SEMA4C (semaphorin 4C), SEMA4D (semaphorin 4D), SEMA4G (semaphorin 4G), SEMA7A 8semaphorin 7A (John Milton Hagen blood group)), SIGIRR (single Ig and TIR domain containing), SIGLEC1 (sialic acid binding Ig like lectin 1), SIGLECS (sialic acid binding Ig like lectin 5), SIGLEC6 (sialic acid binding Ig like lectin 6), SIGLEC7 (sialic acid binding Ig like lectin 7), SIGLEC8 (sialic acid binding Ig like lectin 8), SIGLEC9 (sialic acid binding Ig like lectin 9), SIGLEC10 (sialic acid binding Ig like lectin 10), SIGLEC11 (sialic acid binding Ig like lectin 11), SIGLEC12 (sialic acid binding Ig like lectin 12 (gene/pseudogene)), SIGLEC14 (sialic acid binding Ig like lectin 14), SIGLEC15 (sialic acid binding Ig like lectin 15), SLAMF1 (signaling lymphocytic activation molecule family member 1), SLAMF6 (SLAM family member 6), SLAMF8 (SLAM family member 8), SIRPG; TARM1 (T-cell-interacting, activating receptor on myeloid cells 1), TEK (TEK receptor tyrosine kinase), THY1 Thy-1 cell surface antigen), TIE1 (tyrosine kinase with immunoglobulin like and EGF like domains 1), TMEM81 (transmembrane protein 81), TMIGD1 (transmembrane and immunoglobulin domain containing 1), TMIGD2 (transmembrane and immunoglobulin domain containing 2), TTN (titin), TYRO3 (TYRO3 protein tyrosine kinase), UNC5D, VCAM1 (vascular cell adhesion molecule 1), VSIG1 (V-set and immunoglobulin domain containing 1), VSIG2 (V-set and immunoglobulin domain containing 2), VSIG4 (V-set and immunoglobulin domain containing 4), VSIG10 (V-set and immunoglobulin domain containing 10), VSIG10L (V-set and immunoglobulin domain containing 10 like), VSTM1 (V-set and transmembrane domain containing 1), VTCN1 (V-set domain containing T-cell activation inhibitor 1), ZPBP (zona pellucida binding protein), or ZPBP2 (zona pellucida binding protein 2).

More preferably, the Ig-like domain is an Ig-like domain of any one of the following proteins: CD2, CD3, CD4, CD8, CD19, CD22, CD33, CD80, CD86, in particular of CD4.

Moreover, it is also preferred that the (poly)peptide of interest comprises or consists of one or more antibody domains, such as one or more variable domains (e.g., a light chain variable domain (V_{L}) or a heavy chain variable domain (V_{H})) and/or one or more constant domains (e.g., a light chain constant domain (C_{L}) or one or more (two or three) heavy chain constant domain(s) (C_{H1}, C_{H2}, C_{H3})). For example, the (poly)peptide of interest comprises or consists of a (heterologous) V_{H} domain. In another example, the (poly)peptide of interest comprises or consists of a (heterologous) V_{H} and a (heterologous) V_{L} domain. In another example, the (poly)peptide of interest comprises or consists of two (heterologous) V_{H} and a (heterologous) V_{L} domains (e.g. V_{H}—V_{L}-V_{H}). In another example, the (poly)peptide of interest comprises or consists of a (heterologous) V_{H} domain, a (heterologous) C_{L} domain and a (heterologous) V_{L} domain (e.g. V_{L}—C_{L}-V_{H}). It is also preferred that the antibody domain may be combined with another functional domain as described herein, such as a receptor domain (e.g., a receptor domain and a V_{H} domain).

Further preferred examples of Ig-like domains are described herein below.

Another preferred functional domain (comprised in the (poly)peptide of interest) comprises or consists of an extra- and/or intracellular domain of a (known) protein. Moreover, the functional domain may preferably comprise or consist of a domain of a (known) soluble globular protein. More preferably, the functional domain comprises or consists of an extracellular domain of a (known) protein or a domain of a (known) soluble globular protein.

Preferably, the functional domain (comprised in the (poly)peptide of interest) comprises or consists of a carrier domain, a reporter domain, a tag, a localization domain, an (independent) binding site, an enzyme or enzymatic domain, a receptor or a functional fragment thereof, or a ligand or a functional fragment thereof.

Preferably, the functional domain (comprised in the (poly)peptide of interest) comprises or consists of an enzyme or an enzymatic domain thereof. An “enzyme” is a polypeptide or protein catalyst, i.e. an enzyme typically accelerates a chemical reaction. The molecules upon which enzymes may act are called substrates and the enzyme converts the substrates into different molecules known as products. Almost all metabolic processes in the cell need enzymes in order to occur at rates fast enough to sustain life. Preferred enzymes include oxidoreductases, transferases, hydrolases, lysases, isomerases and ligases. For enzymes, which form a dimer, the (poly)peptide of interest may comprise two identical domains connected by a linker. For example, enzymes may be useful to activate a pro-drug at a specific site, e.g. a tumor. Examples of preferred enzymes and uses of antibodies comprising such enzymes are described in Andrady C, Sharma S K, Chester K A; Antibody-enzyme fusion proteins for cancer therapy; Immunotherapy. 2011 February; 3(2):193-211 and in Boado R P, Zhang Y, Zhang Y, Xia C F, Wang Y, Pardridge W M; Genetic engineering of a lysosomal enzyme fusion protein for targeted delivery across the human blood-brain barrier; Biotechnol Bioeng. 2008 Feb. 1; 99(2):475-84.

Preferred enzymes are selected from the group consisting of dehydrogenase, luciferase, DMSO reductase, Alcohol dehydrogenase (NAD), Alcohol dehydrogenase (NADP), Homoserine dehydrogenase, Aminopropanol oxidoreductase, Diacetyl reductase, Glycerol dehydrogenase, Propanediol-phosphate dehydrogenase, glycerol-3-phosphate dehydrogenase (NAD+), D-xylulose reductase, L-xylulose reductase, Lactate dehydrogenase, Malate dehydrogenase, Isocitrate dehydrogenase, HMG-CoA reductase, Glucose oxidase, L-gulonolactone oxidase, Thiamine oxidase, Xanthine oxidase, Acetaldehyde dehydrogenase, Glyceraldehyde 3-phosphate dehydrogenase, Pyruvate dehydrogenase, Oxoglutarate dehydrogenase, Biliverdin reductase, Monoamine oxidase, Dihydrofolate reductase, Methylenetetrahydrofolate reductase, Sarcosine oxidase, Dihydrobenzophenanthridine oxidase, NADH dehydrogenase, Urate oxidase, Nitrite reductase, Nitrate reductase, Glutathione reductase, Thioredoxin reductase, Sulfite oxidase, Cytochrome c oxidase, Coenzyme Q—cytochrome c reductase, Catechol oxidase, Laccase, Cytochrome c peroxidase, Catalase, Myeloperoxidase, Thyroid peroxidase, Glutathione peroxidase, 4-hydroxyphenyl pyruvate dioxygenase, Renilla-luciferin 2-monooxygenase, Cypridina-luciferin 2-monooxygenase, Firefly luciferase, Watasenia-luciferin 2-monooxygenase, Oplophorus-luciferin 2-monooxygenase, Aromatase, CYP2D6, CYP2E1, CYP3A4, Cytochrome P450 oxidase, Nitric oxide dioxygenase, Nitric oxide synthase, Aromatase, Phenylalanine hydroxylase, Tyrosinase, Superoxide dismutase, Ceruloplasmin, Nitrogenase, Deiodinase, Glutathione S-transferase, Catechol-O-methyl transferase, DNA methyltransferase, Histone methyltransferase, ATCase, Ornithine transcarbamoylase, Aminolevulinic acid synthase, Choline acetyltransferase, Factor XIII, Gamma glutamyl transpeptidase, Transglutaminase, Hypoxanthine-guanine phosphoribosyltransferase, Thiaminase, Alanine transaminase, Aspartate transaminase, Butyrate kinase, Nuclease, Endonuclease, Exonuclease, Acid hydrolase, Phospholipase A, Phospholipase C, Acetylchol inesterase, Cholinesterase, Lipoprotein lipase, Ubiquitin carboxy-terminal hydrolase L1, Phosphatase, Alkaline phosphatase, Fructose bisphosphatase, CGMP specific phosphodiesterase type 5, Phospholipase D, Restriction enzyme Type 1, Restriction enzyme Type 2, Restriction enzyme Type 3, Restriction enzyme Type 4, Deoxyribonuclease I, RNase H, Ribonuclease, Amylase, Sucrase, Chitinase, Lysozyme, Maltase, Lactase, Beta-galactosidase, Hyaluronidase, Adenosylmethionine hydrolase, S-adenosyl-L-homocysteine hydrolase, Alkenylglycerophosphocholine hydrolase, Alkenylglycerophosphoethanolamine hydrolase, Cholesterol-5,6-oxide hydrolase, Hepoxilin-epoxide hydrolase, Isochorismatase, Leukotriene-A4 hydrolase, Limonene-1,2-epoxide hydrolase, Microsomal epoxide hydrolase, Trans-epoxysuccinate hydrolase, Alanine aminopeptidase, Angiotensin converting enzyme, Serine protease, Chymotrypsin, Trypsin, Thrombin, Factor X, Plasmin, Acrosin, Factor VII, Factor IX, Prolyl oligopeptidase, Factor XI, Elastase, Factor XII, Proteinase K, Tissue plasminogen activator, Protein C, Separase, Pepsin, Rennet, Renin, Trypsinogen, Plasmepsin, Matrix metalloproteinase, Metalloendopeptidase, Urease, Beta-lactamase, Arginase, Adenosine deaminase, GTP cyclohydrolase I, Nitrilase, Helicase, DnaB helicase, RecQ helicase, ATPase, NaKATPase, ATP synthase, Kynureninase, Haloacetate dehalogenase, Lyase, Ornithine decarboxylase, Uridine monophosphate synthetase, Aromatic-L-amino-acid decarboxylase, RubisCO, Carbonic anhydrase, Tryptophan synthase, Phenylalanine ammonia-lyase, Cystathionine gamma-lyase, Cystathionine beta-lyase, Leukotriene C4 synthase, Dichloromethane dehalogenase, Halohydrin dehalogenase, Adenylate cyclase, Guanylate cyclase, Amino-acid racemase: Phenylalanine racemase, Serine racemase, Mandelate racemase, UDP-glucose 4-epimerase, Methylmalonyl CoA epimerase, FKBP: FKBP1A, FKBP1B, FKBP2, FKBP3, FKBP5, FKBP6, FKBP8, FKBP9, FKBP10, FKBP52, FKBPL, Cyclophilin, Parvulin, Prolyl isomerase, 2-chloro-4-carboxymethylenebut-2-en-1,4-olide isomerase, Beta-carotene isomerase, Farnesol 2-isomerase, Furylfuramide isomerase, Linoleate isomerase, Maleate isomerase, Maleylacetoacetate isomerase, Maleylpyruvate isomerase, Parvulin, Photoisomerase, Prolycopene isomerase, Prolyl isomerase, Retinal isomerase, Retinol isomerase, Zeta-carotene isomerase, Enoyl CoA isomerase, Protein disulfide isomerase, Phosphoglucomutase, Muconate cycloisomerase, 3-carboxy-cis,cis-muconate cycloisomerase, Tetrahydroxypteridine cycloisomerase, Inositol-3-phosphate synthase, Carboxy-cis,cis-muconate cyclase, Chalcone isomerase, Chloromuconate cycloisomerase, (+)-bornyl diphosphate synthase, Cycloeucalenol cycloisomerase, Alpha-pinene-oxide decyclase, Dichloromuconate cycloisomerase, Copalyl diphosphate synthase, Ent-copalyl diphosphate synthase, Syn-copalyl-diphosphate synthase, Terpentedienyl-di phosphate synthase, Halimadienyl-diphosphate synthase, (S)-beta-macrocarpene synthase, Lycopene epsilon-cyclase, Lycopene beta-cyclase, Prosolanapyrone-III cycloisomerase, D-ribose pyranase, Steroid Delta Isomerase, Topoisomerase, 6-carboxytetrahydropterin synthase, FARSB, Glutamine synthetase, CTP synthase, Argininosuccinate synthetase, Pyruvate carboxylase, Acetyl-CoA carboxylase, and DNA ligase.

More preferred enzymes may be selected from the group consisting of carboxypeptidase, lactamase, cytosine deaminase, β-glucuronidase, purine nucleoside phosphorylase, granzyme B, caspase and RNase, such as HPR (human pancreatic RNase, barnase, bovine seminal RNase, onconase, RapLR1, angiogenin, dicer, DIS3-like exonuclease 2, phosphodiesterase ELAC 2, RNase HIII, RNase T2, and tRNA splicing ribonuclease.

A functional fragment of an enzyme may be any fragment of an enzyme, which has the ability to mediate a functionality. Usually, such fragments are referred to as “domains”. Accordingly, the functional fragment of an enzyme may be any domain of the enzyme. Preferred examples include functional fragments (e.g., domains) of the (exemplified) enzymes described above. Preferably, the functional fragment of the enzyme, which is comprised by the functional domain is a catalytic domain of an enzyme. The catalytic domain of an enzyme is the region of an enzyme that interacts with its substrate to cause the enzymatic reaction. For example, the functional domain may be a catalytic domain of any one of the following enzymes: carboxypeptidase, β-lactamase, cytosine deaminase, β-glucuronidase, purine nucleoside phosphorylase, granzyme B, caspase and RNase, such as HPR (human pancreatic RNase, barnase, bovine seminal RNase, onconase, RapLR1, angiogenin, dicer, DIS3-like exonuclease 2, phosphodiesterase ELAC 2, RNase H111, RNase T2, and tRNA splicing ribonuclease.

Preferably, the functional domain (comprised in the (poly)peptide of interest) comprises or consists of a carrier domain. As used herein, a “carrier domain” refers to an amino acid sequence, which provides for conjugation of the antibody to another molecule. In a preferred example, the carrier domain provides for conjugation of the antibody, or the antigen binding fragment thereof, for example to a drug, to an imaging agent, or to a nanoparticle. In general, preferred examples of conjugates, which may be useful in the context of the present invention, are described in Wu, A. M., and Senter, P. D. (2005) Arming antibodies: Prospects and challenges for immunoconjugates. Nat. Biotechnol. 23, 1137-1146.

For example, drugs, which may be conjugated to the antibody include anticancer drugs, such as those described in Thomas A, Teicher B A, Hassan R; Antibody-drug conjugates for cancer therapy; Lancet Oncol. 2016 June; 17(6):e254-62. doi: 10.1016/S1470-2045(16)30030-4. For example, imaging agents, which may be conjugated to the antibody, are described in Steve Knutson, Erum Raja, Ryan Bomgarden, Marie Nlend, Aoshuang Chen, Ramaswamy Kalyanasundaram, and Surbhi Desai; Development and Evaluation of a Fluorescent Antibody-Drug Conjugate for Molecular Imaging and Targeted Therapy of Pancreatic Cancer; PLoS One 2016; 11(6): e0157762. Such drugs are preferably cytotoxic agents. Preferred examples of drugs, which may be conjugated to the antibody or antigen binding fragment of the present invention, include doxorubicin, truncated Pseudomonas exotoxin A, maytansinoid DM1.

Examples of imaging agents, which may be conjugated to the antibody or antigen binding fragment of the present invention, include radioisotopes, such as those described in Schubert M, Bergmann R, Förster C, Sihver W, Vonhoff S, Klussmann S, Bethge L, Walther M, Schlesinger J, Pietzsch J, Steinbach J, Pietzsch H J; Novel Tumor Pretargeting System Based on Complementary I-Configured Oligonucleotides; Bioconjug Chem. 2017 Apr. 19; 28(4):1176-1188 and in Bhusari P, Vatsa R, Singh G; Parmar M, Bal A, Dhawan D K, Mittal B R, Shukla J; Development of Lu-177-trastuzumab for radioimmunotherapy of HER2 expressing breast cancer and its feasibility assessment in breast cancer patients; Int J Cancer. 2017 Feb. 15; 140(4):938-947. Preferred examples of radioisotopes include ⁹⁰Y, ¹³¹I, and ¹⁷⁷Lu.

Further examples of imaging agents, which may be conjugated to the antibody or antigen binding fragment of the present invention, include fluorescent dyes, quantum dots, and iron oxide. Examples of fluorescent dyes include those described below as reporter domains. An example of iron oxide nanoparticles is described in Hengyi Xu, Zoraida P. Aguilar, Lily Yang, Min Kuang, Hongwei Duan, Yonghua Xiong, Hua Wei, and Andrew Wang: Antibody Conjugated Magnetic Iron Oxide Nanoparticles for Cancer Cell Separation in Fresh Whole Blood. Biomaterials. 2011 December; 32(36): 9758-9765.

Antibody conjugates (i.e. antibodies conjugated to other molecules) are known in the art. In particular, the molecule conjugated to the antibody may be linked to the antibody by a cleavable or non-cleavable linker (e.g., as described in: Thomas H. Pillow. Novel linkers and connections for antibody-drug conjugates to treat cancer and infectious disease. Pharmaceutical Patent Analyst Vol. 6, No. 1, Feb. 3, 2017, https://doi.org/10.4155/ppa-2016-0032; or in: Beck A, Goetsch L, Dumontet C, Corvga N. Strategies and challenges for the next generation of antibody-drug conjugates. Nat Rev Drug Discov. 2017 May; 16(5):315-337). Examples of such linkers, which may be used to link the molecule to the antibody or antigen binding fragment, are described, for example in EP 2927227 and in Thomas H. Pillow. Novel linkers and connections for antibody-drug conjugates to treat cancer and infectious disease. Pharmaceutical Patent Analyst Vol. 6, No. 1, Feb. 3, 2017, https://doi.org/10.4155/ppa-2016-0032. However, in the prior art the linkers are attached directly to the Ig-domains of the antibody (namely, to the variable and/or constant domains of the antibody), which may interfere with the functionality of the Ig-domains of the antibody. In view thereof, the functional domain may be used for attachment of a linker to the antibody. Preferred linkers differ from “classical” linkers in that they are engineered to contain additional cysteines or lysines. Preferably, the carrier domain comprises one or more non-canonical amino acids useful for site-specific conjugation, e.g., as described in Link A J, Mock M L, Tirrell D A. Non-canonical amino acids in protein engineering. Curr Opin Biotechnol. 2003 December; 14(6):603-9. Moreover, the carrier domain may be designed such that it is recognized by specific enzymes (such as Formylglycin Generating Enzyme, Sortase and/or Transglutaminases), that modify specific amino acids that then can be used for conjugation as described in section 6 of Dennler P., Fischer E., Schibli R. Antibody conjugates: From heterogeneous populations to defined reagents. Antibodies. 2015; 4:197-224.

Further preferred carrier domains are domains for conjugation, such as genetically modified cross-reacting material (CRM) of diphtheria toxin, tetanus toxoid (T), meningococcal outer membrane protein complex (OMPC), diphtheria toxoid (D), and H. influenzae protein D (HiD), for example as described in Pichichero M E: Protein carriers of conjugate vaccines: characteristics, development, and clinical trials, Hum Vaccin Immunother. 2013 December; 9(12):2505-23.

Preferably, the functional domain (comprised in the (poly)peptide of interest) comprises or consists of a reporter domain. A reporter domain is typically encoded by a reporter gene. Reporter domains are such domains, whose presence (e.g., in a cell, organism) can be easily observed. Reporter domains include, for example, fluorescent proteins, such as GFP/EGFP (green fluorescent protein/enhanced green fluorescent protein), YFP (yellow fluorescent protein), RFP (red fluorescent protein, such as tdTomato or DsRed), and CFP (cyan fluorescent protein), luciferases and enzymes such as beta-galactosidase and peroxidase. Reporter domains can be useful for in vivo and ex vivo approaches. For example, fluorescent proteins cause a cell to fluoresce when excited with light of a particular wavelength, luciferases cause a cell to catalyze a reaction that produces light, and enzymes such as beta-galactosidase convert a substrate to a colored product. There are several different ways to measure or quantify a reporter depending on the particular reporter and what kind of characterization data is desired. In general, microscopy is useful for obtaining both spatial and temporal information on reporter activity, particularly at the single cell level. Flow cytometers are best suited for measuring the distribution in reporter activity across a large population of cells. Plate readers are generally best for taking population average measurements of many different samples over time. Enzyme, such as beta-galactosidase and peroxidase, which can react to a given substrate, may be useful, for example, for ex-vivo stainings of human samples, e.g. in tumor diagnosis. However, in some embodiments the (poly)peptide of interest does not comprise GFP (green fluorescent protein) or RFP (red fluorescent protein, such as tdTomato or DsRed). In more general, in some embodiments the (poly)peptide of interest does not comprise a fluorescent (reporter) protein. Accordingly, in some embodiments the DNA molecule does not comprise a nucleotide sequence encoding GFP or RFP (or, in more general, a fluorescent (reporter) protein.

Preferably, the reporter domain comprises or consists of an amino acid sequence coding for GFP/EGFP, YFP, RFP, CFP, luciferase, beta-galactosidase, or peroxidase. In addition, fluorescent tags as described below are also useful as reporter domains.

Preferably, the functional domain (comprised in the (poly)peptide of interest) comprises or consists of a localization domain. In general, a localization domain directs a protein to a certain target, e.g. on the level of an organism or a cell. A localization domain can direct the antibody or the antigen binding fragment according to the present invention to a particular physical location in the cell, such as the nucleus, the membrane, the periplasm, secretion outside of the cell, to a specific part of the body, or elsewhere.

For example, in order to direct the antibody or the antigen binding fragment according to the present invention into a cell, the functional domain may comprise or consist of a cell penetrating peptide. The term “cell penetrating peptides” (“CPPs”, also referred to as “protein transduction domain”/“PTD”) is generally used to designate short peptides that are able to transport different types of cargo molecules across plasma membrane, and, thus, facilitate cellular uptake of various molecular cargoes (from nanosize particles to small chemical molecules and large fragments of DNA). Cell penetrating peptides typically have an amino acid composition that either contains a high relative abundance of positively charged amino acids such as lysine or arginine or have a sequence that contains an alternating pattern of polar/charged amino acids and non-polar, hydrophobic amino acids. These two types of structures are referred to as polycationic or amphipathic, respectively. Typically, cell penetrating peptides (CPPs) are peptides of 8 to 50 residues that have the ability to cross the cell membrane and enter into most cell types. Alternatively, they are also called protein transduction domain (PTDs) reflecting their origin as occurring in natural proteins. Frankel and Pabo simultaneously to Green and Lowenstein described the ability of the trans-activating transcriptional activator from the human immunodeficiency virus 1 (HIV-TAT) to penetrate into cells (Frankel, A. D. and C. O. Pabo, Cellular uptake of the t at protein from human immunodeficiency virus. Cell, 1988. 55(6): p. 1189-93). In 1991, transduction into neural cells of the Antennapedia homeodomain (DNA-binding domain) from Drosophila melanogaster was described (Joliot, A., et al., Antennapedia homeobox peptide regulates neural morphogenesis. Proc Natl Acad Sci USA, 1991. 88(5): p. 1864-8). In 1994, the first 16-mer peptide CPP called Penetratin was characterized from the third helix of the homeodomain of Antennapedia (Derossi, D., et al., The third helix of the Antennapedia homeodomain translocates through biological membranes. J Biol Chem, 1994. 269(14): p. 10444-50), followed in 1998 by the identification of the minimal domain of TAT, required for protein transduction (Vives, E., P. Brodin, and B. Lebleu, A truncated HIV-1 Tat protein basic domain rapidly translocates through the plasma membrane and accumulates in the cell nucleus. J Biol Chem, 1997. 272(25): p. 16010-7). Over the past two decades, dozens of peptides were described from different origins including viral proteins, e.g. VP22 (Elliott, G. and P. O'Hare, Intercellular trafficking and protein delivery by a herpesvirus structural protein. Cell, 1997. 88(2): p. 223-33), or from venoms, e.g. melittin (Dempsey, C. E., The actions of melittin on membranes. Biochim Biophys Acta, 1990. 1031(2): p. 143-61), mastoporan (Konno, K., et al., Structure and biological activities of eumenine mastoparan-AF (EMP-AF), a new mast cell degranulating peptide in the venom of the solitary wasp (Anterhynchium flavomarginatum micado). Toxicon, 2000. 38(11): p. 1505-15), maurocalcin (Esteve, E., et al., Transduction of the scorpion toxin maurocalcine into cells. Evidence that the toxin crosses the plasma membrane. J Biol Chem, 2005. 280(13): p. 12833-9), crotamine (Nascimento, F. D., et al., Crotamine mediates gene delivery into cells through the binding to heparan sulfate proteoglycans. J Biol Chem, 2007. 282(29): p. 21349-60) or buforin (Kobayashi, S., et al., Membrane translocation mechanism of the antimicrobial peptide buforin 2. Biochemistry, 2004. 43(49): p. 15610-6). Synthetic CPPs were also designed including the poly-arginine (R8, R9, R10 and R12) (Futaki, S., et al., Arginine-rich peptides. An abundant source of membrane-permeable peptides having potential as carriers for intracellular protein delivery. J Biol Chem, 2001. 276(8): p. 5836-40) or transportan (Pooga, M., et al., Cell penetration by transportan. FASEB J, 1998. 12(1): p. 67-77). Any of the above described CPPs may be used as cell penetrating peptide in the antibody or antigen binding fragment according to the present invention. Various CPPs, which can be used as cell penetrating peptide in the antibody or antigen binding fragment according to the present invention are also disclosed in the review: Milletti, F., Cell-penetrating peptides: classes, origin, and current landscape. Drug Discov Today 17 (15-16): 850-60, 2012.

Another example of a localization domain, which may be used in the antibody or antigen binding fragment according to the present invention is a domain for crossing the blood brain barrier, for example as described in Farrington G K, Caram-Salas N, Haqqani A S, Brunette E, Eldredge J, Pepinsky B, Antognetti G, Baumann E, Ding W, Garber E, Jiang S, Delaney C, Boileau E, Sisk W P, Stanimirovic D B. A novel platform for engineering blood-brain barrier-crossing bispecific biologics. FASEB J. 2014 November; 28(11):4764-78.

A further example of a localization domain is a nuclear localization domain. A nuclear localization domain directs a protein, in particular the antibody or antigen-binding fragment according to the present invention, to the cell nucleus. A nuclear localization domain may be useful for an antibody or antigen-binding fragment to block the activity of a transcription factor and to modulate gene expression. Preferred examples of nuclear localization domains are described in Kalderon D, Roberts B L, Richardson W D, Smith A E (1984) “A short amino acid sequence able to specify nuclear location” Cell 39 (3 Pt 2): 499-509 and in Lusk C P, Blobel G, King M C (May 2007) “Highway to the inner nuclear membrane: rules for the road” Nature Reviews Molecular Cell Biology 8 (5): 414-20.

Preferably, the functional domain (comprised in the (poly)peptide of interest) comprises or consists of a tag. More preferably, the tag is an affinity tag, a solubilization tag, a chromatography tag, an epitope tag or a fluorescence tag.

A tag is a peptide sequence grafted onto a recombinant protein. Examples of tags include affinity tags, solubilization tags, chromatography tags, epitope tags, fluorescence tags and protein tags. Affinity tags may be used to purify proteins from their crude biological source using an affinity technique. Examples of affinity tags include chitin binding protein (CBP), maltose binding protein (MBP), and glutathione-S-transferase (GST). A further example is the poly(His) tag which binds to metal matrices. Solubilization tags may be used, especially for recombinant proteins expressed in chaperone-deficient species such as E. coli, to assist in the proper folding in proteins and keep them from precipitating. Examples of solubilization tags include thioredoxin (TRX) and poly(NANP). Chromatography tags may be used to alter chromatographic properties of the protein to afford different resolution across a particular separation technique. Chromatography tags often consist of polyanionic amino acids, such as FLAG-tag. Epitope tags are short peptide sequences which are chosen because high-affinity antibodies can be reliably produced in many different species. These are usually derived from viral genes, which explain their high immunoreactivity. Epitope tags include V5-tag, Myc-tag, HA-tag and NE-tag. These tags are particularly useful for western blotting, immunofluorescence and immunoprecipitation experiments, although they also find use in antibody purification. Fluorescence tags may be used to give visual readout on a protein. GFP and its variants are the most commonly used fluorescence tags. GFP may be used as a folding reporter (fluorescent if folded, colorless if not). Protein tags may allow specific enzymatic modification (such as biotinylation by biotin ligase) or chemical modification (such as reaction with FlAsH-EDT2 for fluorescence imaging). Tags may be combined, for example, in order to connect proteins to multiple other components. Tags may be removable by chemical agents or by enzymatic means, such as proteolysis or intein splicing.

Preferred examples of tags include, but are not limited to, the following: twin-Strep-Tag (SAWSHPQFEKGGGSGGGSGGSAWSHPQFEK; SEQ ID NO: 65); AviTag, a peptide allowing biotinylation by the enzyme BirA and so the protein can be isolated by streptavidin (GLNDIFEAQKIEWHE; SEQ ID NO: 66); Calmodulin-tag, a peptide bound by the protein calmodulin (KRRWKKNFIAVSAANRFKKISSSGAL; SEQ ID NO: 67); polyglutamate tag, a peptide binding efficiently to anion-exchange resin such as Mono-Q (EEEEEE; SEQ ID NO: 68); E-tag, a peptide recognized by an antibody (GAPVPYPDPLEPR; SEQ ID NO: 69); FLAG-tag, a peptide recognized by an antibody (DYKDDDDK; SEQ ID NO: 70); HA-tag, a peptide from hemagglutinin recognized by an antibody (YPYDVPDYA; SEQ ID NO: 71); His-tag, 5-10 histidines bound by a nickel or cobalt chelate (HHHHHH; SEQ ID NO: 72); Myc-tag, a peptide derived from c-myc recognized by an antibody (EQKLISEEDL; SEQ ID NO: 73); NE-tag, an 18-amino-acid synthetic peptide (TKENPRSNQEESYDDNES; SEQ ID NO: 74) recognized by a monoclonal IgG1 antibody, which is useful in a wide spectrum of applications including Western blotting, ELISA, flow cytometry, immunocytochemistry, immunoprecipitation, and affinity purification of recombinant proteins; S-tag, a peptide derived from Ribonuclease A (KETAAAKFERQHMDS; SEQ ID NO: 75); SBP-tag, a peptide which binds to streptavidin (MDEKTTGWRGGHVVEGLAGELEQLRARLEHHPQGQREP; SEQ ID NO: 76); Softag 1, for mammalian expression (SLAELLNAGLGGS; SEQ ID NO: 77); Softag 3, for prokaryotic expression (TQDPSRVG; SEQ ID NO: 78); Strep-tag, a peptide which binds to streptavidin or the modified streptavidin called streptactin (Strep-tag 11: WSHPQFEK; SEQ ID NO: 79); TC tag, a tetracysteine tag that is recognized by FlAsH and ReAsH biarsenical compounds (CCPGCC; SEQ ID NO: 80); V5 tag, a peptide recognized by an antibody (GKPIPNPLLGLDST; SEQ ID NO: 81); VSV-tag, a peptide recognized by an antibody (YTDIEMNRLGK; SEQ ID NO: 82); Xpress tag (DLYDDDDK; SEQ ID NO: 83); Isopeptag, a peptide which binds covalently to pilin-C protein (TDKDMTITFTNKKDAE; SEQ ID NO: 84); SpyTag, a peptide which binds covalently to SpyCatcher protein (AHIVMVDAYKPTK; SEQ ID NO: 85); SnoopTag, a peptide which binds covalently to SnoopCatcher protein (KLGDIEFIKVNK; SEQ ID NO: 86); Ty1 tag (EVHTNQDPLD; SEQ ID NO: 87); BCCP (Biotin Carboxyl Carrier Protein), a protein domain biotinylated by BirA enabling recognition by streptavidin; glutathione-S-transferase (GST)-tag, a protein which binds to immobilized glutathione; green fluorescent protein-tag, a protein which is spontaneously fluorescent and can be bound by nanobodies; HaloTag, a mutated bacterial haloalkane dehalogenase that covalently attaches to a reactive haloalkane substrate, this allows attachment to a wide variety of substrates; maltose binding protein (MBP)-tag, a protein which binds to amylose agarose; Nus (N-utilization substance)-tag; Thioredoxin (Trx)-tag; Fasciola hepatica 8-kDa antigen (Fh8)-tag; Small ubiquitin modified (SUMO)-tag; Solubility-enhancer peptide sequences (SET)-tags; IgG domain B1 of Protein G (GB1)-tag; IgG repeat domain ZZ of Protein A (ZZ)-tag; Solubility eNhancing Ubiquitous Tag (SNUT)-tag; Seventeen kilodalton protein (Skp)-tag; Phage T7 protein kinase (T7PK)-tag; E. coli secreted protein A (EspA)-tag; Monomeric bacteriophage T7 0.3 protein (Orc protein)/Mocr-tag; E. coli trypsin inhibitor (Ecotin)-tag;

Calcium-binding protein (CaBP)-tag; Stress-responsive arsenate reductase (ArsC)-tag; N-terminal fragment of translation initiation factor IF2 (IF2-domain I)-tag; Expressivity tag (N-terminal fragment of translation initiation factor IF2); Stress-responsive proteins RpoA, SlyD, Tsf, RpoS, PotD, Crr-tags; E. coli acidic proteins msyB, yjgD, rpoD tags (see, e.g., Costa S, Almeida A, Castro A, Domingues L. Fusion tags for protein solubility, purification and immunogenicity in Escherichia coli: the novel Fhb system. Frontiers in Microbiology. 2014; 5:63, in particular Table 1 in Costa et al., 2014).

Accordingly, it is preferred that the tag comprises or consists of an amino acid sequence according to any one of SEQ ID NO: 65-87 or a sequence variant thereof. Most preferably, the tag is a Strep-tag, in particular according to SEQ ID NO: 65 or 79.

Preferably, the functional domain (comprised in the (poly)peptide of interest) comprises or consists of a receptor or a functional fragment thereof (also referred to as “receptor domain”). A “receptor” is a polypeptide or protein, which binds a specific (signal) molecule, its ligand, and which may initiate a response, e.g. in a cell. In nature, receptors are in particular located on or in the cell membrane (cell surface receptors) or intracellularly (intracellular receptors). Preferred receptors include ion channel-linked (ionotropic) receptors, G protein-linked (metabotropic) hormone receptors, and enzyme-linked hormone receptors, cytoplasmic receptors and nuclear receptors. For receptors, which form a dimer, the (poly)peptide of interest may comprise two identical domains connected by a linker.

Preferred receptors are receptors comprising an Ig-like domain. In particular, the receptor may be an inhibitor receptor comprising an Ig-like domain or an activating receptor comprising an Ig-like domain. Preferred examples of inhibitory receptors comprising an Ig-like domain include programmed cell death protein 1 (PD-1 or PD1), cytotoxic T-lymphocyte-associated protein 4 (CTLA4), B- and T-lymphocyte attenuator (BTLA), T-cell immunoglobulin and mucin-domain containing-3 (TIM-3; also known as Hepatitis A virus cellular receptor 2 (HAVCR2)), T cell immunoreceptor with Ig and ITIM domains (TIGIT), Cell surface glycoprotein CD200 receptor 1 (CD200R1), 2B4 (CD244; SLAMF4), Trem (Triggering receptor expressed on myeloid cells)-like transcript 2 (TLT2), Leukocyte immunoglobulin-like receptor subfamily B member 4 (LILRB4), and Killer Cell Immunoglobulin Like Receptor, Two Ig Domains And Long Cytoplasmic Tail 2 (KIR2DL2). Preferred examples of activating receptors comprising an Ig-like domain include Inducible T-cell COStimulator (ICOS) and CD28. Particularly preferably, the receptor is programmed cell death protein 1 (PD-1 or PD1) or Signaling lymphocytic activation molecule (SLAM).

Further preferred receptors are soluble receptors, for example as disclosed in Heaney M L, Golde D W. Soluble receptors in human disease. J Leukoc Biol. 1998 August; 64(2):135-46. Examples thereof include TNFR (tumor necrosis factor receptor), p55, p75, Fas (CD95), nerve growth factor receptor, CD27, CD30, growth hormone receptor, GM-CSF receptor, erythropoietin receptor (EpoR), thrombopoietin receptor, G-CSF receptor, IL-1RI (interleukin 1 receptor I), IL-1R11 (interleukin 1 receptor II), IL-2Ra (interleukin 2 receptor a, Tac, CD25), IL-4R (interleukin 4 receptor), IL-5Ra (interleukin 5 receptor a), IL-7R (interleukin 7 receptor), IL-6Ra (interleukin 6 receptor a), gp130, CNTFR (ciliary neurotrophic factor receptor), LIFR (leukemia inhibitory factor receptor), leptin receptor, IL-11R (interleukin 11 receptor), IL-12 p40 (interleukin 12 receptor p40), stem cell factor receptor (c-kit), interferon receptor, lipopolysaccharide receptor (CD14), complement receptor type I (CD35), hyaluronate receptor (CD44), CD58, IgE receptor (FcεRII, CD23), IgG receptor (FcγRII), ICAM-1 (CD54), ICAM-3 (CD50), transforming growth factor 13 receptor III, epidermal growth factor receptor (c-erb B), vascular endothelial growth factor receptor, platelet derived growth factor receptor, fibroblast growth factor, colony stimulating factor-1 receptor (MCFR, c-fms), ARK (adrenergic receptor kinase), Tie (angiopoietin receptor), insulin receptor, insulin-like growth factor-II receptor, and mannose 6-phosphate receptor.

More preferably, the soluble receptor is a soluble cytokine receptor, such as a class I cytokine receptor superfamily receptor, a class II cytokine receptor superfamily receptor, an IL-1/TLR family receptor, a TGF-β receptor family receptor, a TNFR superfamily receptor, or IL-17R. Preferred receptors of class I cytokine receptor superfamily include IL-4Rα, IL-5Rα, IL-6Rα, IL-7Rα, IL-9Rα, EpoR, G-CSFR, GM-CSFRα, gp130, and LIFRα. Preferred receptors of class II cytokine receptor superfamily include type I IFNR, such as IFNAR1 and IFNAR2a. Preferred receptors of IL-1/TLR family include IL-1RII and IL-1RacP. Preferred receptors of TGF-β receptor family include TβR-I and activin receptor-like kinase 7. Preferred receptors of the TNFR superfamily include TNFRSF6/Fas/CD95 and TNFRSF9/4-1BB/CD137. Accordingly, preferred examples of cytokine receptors include IL-4Rα, IL-5Rα, IL-6Rα, IL-7Rα, IL-9Rα, EpoR, G-CSFR, GM-CSFRα, gp130, LIFRα, IFNAR1, IFNAR2α, IL-1RII, IL-1RacP, TβR-I, activin receptor-like kinase 7, TNFRSF6/Fas/CD95, TNFRSF9/4-1BB/CD137 and IL-17R. An antibody or antibody fragment comprising a functional domain comprising such a receptor or a functional fragment thereof may modulate the inflammatory response while the antibody reaches its target. For example, soluble type II IL-1 receptors (sIL-1RII), which are generated primarily by proteolytic cleavage in response to a variety of stimuli, can attenuate excessive IL-1 bioactivity by preferentially binding IL-1β. For example, soluble IL-1 RAcP, which is generated by alternative splicing rather than by ectodomain cleavage. For example, soluble IL-6 receptors bind IL-6 with an affinity similar to the membrane IL-6R, thereby prolonging the IL-6 half-life.

A functional fragment of a receptor may be any fragment of a receptor, which has the ability to mediate a functionality. Usually, such fragments are referred to as “domains”. Accordingly, the functional fragment of a receptor may be any domain of the receptor. Preferred examples include functional fragments (e.g., domains) of the (exemplified) receptors described above. Preferably, the functional fragment of the receptor, which is comprised by the functional domain is an extracellular domain of a receptor. For example, the functional domain may be an extracellular domain of any of the following receptors IL-4Rα, IL-5Rα, IL-6Rα, IL-7Rα, IL-9Rα, EpoR, G-CSFR, GM-CSFRα, gp130, LIFRα, IFNAR1, IFNAR2α, IL-1RII, IL-1 RacP, TβR-I, activin receptor-like kinase 7, TNFRSF6/Fas/CD95, TNFRSF9/4-1BB/CD137, IL-17R, p55, p75, nerve growth factor receptor, CD27, CD30, growth hormone receptor, thrombopoietin receptor, IL-1RI (interleukin 1 receptor I), IL-2Ra (interleukin 2 receptor a, Tac, CD25), CNTFR (ciliary neurotrophic factor receptor), leptin receptor, IL-11R (interleukin 11 receptor), IL-12 p40 (interleukin 12 receptor p40), stem cell factor receptor (c-kit), interferon receptor, lipopolysaccharide receptor (CD14), complement receptor type I (CD35), hyaluronate receptor (CD44), CD58, IgE receptor (FcεRII, CD23), IgG receptor (FcγRII), ICAM-1 (CD54), ICAM-3 (CDSO), transforming growth factor β receptor III, epidermal growth factor receptor (c-erb B), vascular endothelial growth factor receptor, platelet derived growth factor receptor, fibroblast growth factor, colony stimulating factor-1 receptor (MCFR, c-fms), ARK (adrenergic receptor kinase), Tie (angiopoietin receptor), insulin receptor, insulin-like growth factor-II receptor, and mannose 6-phosphate receptor.

Preferably, the functional fragment of the receptor, which is comprised by the functional domain is an Ig-like domain. For example, the functional domain may be an Ig-like domain of any of the following receptors PD1, SLAM, LAIR1, CTLA4, BTLA, TIM-3, TIGIT, CD200R1, 2B4 (CD244), TLT2, LILRB4, KIR2DL2, ICOS or CD28. Preferably, the functional domain does not comprise a transmembrane domain. Most preferably, the receptor comprises or consists of (a fragment of) PD1, SLAM, or LAIR1, such as an amino acid sequence as set forth in any one of SEQ ID NOs 88-90 or a sequence variant thereof.

Moreover, it is particularly preferred that the functional domain comprises or consists of a mutated Leukocyte-associated immunoglobulin-like receptor 1 (LAIR1) fragment as described in WO 2016/207402 A1. The mutated LAIR1 fragment as set forth in SEQ ID NO: 88, or a sequence variant thereof having at least 70%, preferably at least 75%, more preferably at least 80%, even more preferably at least 85%, still more preferably 90%, particularly preferably 95%, and most preferably at least 98% sequence identity, is most preferred.

Mutated LAIR1 Fragment:

[SEQ ID NO: 88]
EDLPRPSISAEPGTVIPLGSHVTFVCRGPVGVQTFRLERERNYLYSDTED
VSQTSPSESEARFRIDSVNAGNAGLFRCIYYKSRKWSEQSDYLELVVK

Particularly preferably, the functional domain (comprised in the (poly)peptide of interest) comprises or consists of an Ig-like fragment of PD1 or SLAM, such as an amino acid sequence as set forth in SEQ ID NO: 89 or in SEQ ID NO: 90; or a sequence variant thereof having at least 70%, preferably at least 75%, more preferably at least 80%, even more preferably at least 85%, still more preferably 90%, particularly preferably 95%, and most preferably at least 98% sequence identity.

Pd-1 Fragment:

[SEQ ID NO: 89]
DSPDRPWNPPTFSPALLVVTEGDNATFTCSFSNTSESFVLNWYRMSPSNQ
TDKLAAFPEDRSQPGQDCRFRVTQLPNGRDFHMSVVRARRNDSGTYLCGA
ISLAPKAQIKESLRAELRVT

Slam Fragment:

[SEQ ID NO: 90]
EQVSTPEIKVLNKTQENGTCTLILGCTVEKGDHVAYSWSEKAGTHPLNPA
NSSHLLSLTLGPQHADNIYICTVSNPISNNSQTFSPWPGCRTDPS

Preferably, the functional domain (comprised in the (poly)peptide of interest) comprises or consists of a ligand or a functional fragment thereof. A “ligand” is a molecule, which specifically binds to a specific site on a protein or any other molecule. In the context of the present invention, the ligand is a peptide, polypeptide or protein, since it is comprised in the (poly)peptide of interest. Binding of a ligand occurs in particular by intermolecular forces, such as ionic bonds, hydrogen bonds and Van der Waals forces. Preferred examples of ligands are cytokines and ligands of any one of the receptors described above, in particular of the receptors PD1, SLAM, LAIR1, CTLA4, BTLA, TIM-3, TIGIT, CD200R1, 2B4 (CD244), TLT2, LILRB4, KIR2DL2, ICOS or CD28, such as PD-L1, PD-L2, B7-1, B7-2, B7-H4 (B7 homolog), galectin-9, poliovirus receptor (PVR), OX-2 membrane glycoprotein, CD48, B7-H3 (B7 homolog), MHCI, and ICOS-L.

Preferably, the ligand is a cytokine or a functional fragment thereof. Cytokines are usually small proteins (˜5-20 kDa) that are important in cell signaling. They are released by cells and affect the behavior of other cells, and sometimes affect the behavior of the releasing cell itself. A cytokine may be selected from chemokines such as the SIS family of cytokines, the SIG family of cytokines, the SCY family of cytokines, the Platelet factor-4 superfamily and intercrines, CC chemokine ligands (CCL)-1 to -28 (in particular CCL12), CXCL1-CXCL17, XCL1 (lymphotactin-α) and XCL2 (Iymphotactin-β), fractalkine (or CX₃CL1); interferons, such as Type I IFN, Type II IFN, and Type III IFN, in particular IFN-α, IFN-β, IFN-γ, IFN-ε, IFN-κ, IFN-ω, IL10R2 (also called CRF2-4) and IFNLR1 (also called CRF2-12); interleukins, such as IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, and IL-36; lymphokines, such as IL-2, IL-3, IL-4, IL-5, IL-6, GM-CSF, and Interferon-gamma; tumor necrosis factors, such as CD40LG (TNFSF5); CD70 (TNFSF7); EDA; FASLG (TNFSF6); LTA (TNFSF1); LTB (TNFSF3); TNF, TNFα, TNFSF4 (OX40L); TNFSF8 (CD153); TNFSF9; TNFSF10 (TRAIL); TNFSF11 (RANKL); TNFSF12 (TWEAK); TNFSF13; TNFSF13B; TNFSF14; TNFSF15; and TNFSF18; and colony stimulating factors, such as CSF1 (also known as “macrophage colony-stimulating factor”), CSF2 (also known as “granulocyte macrophage colony-stimulating factor”; GM-CSF and sargramostim), CSF3 (also known as “granulocyte colony-stimulating factor”; G-CSF and filgrastim), as well as synthetic CSFs, such as Promegapoietin. Accordingly, preferred examples of cytokines include IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, IL-36, CCL-1, CCL-2, CCL-3, CCL-4, CCL-5, CCL-6, CCL-7, CCL-8, CCL-9, CCL-10, CCL-11, CCL-12, CCL-13, CCL-14, CCL-15, CCL-16, CCL-17, CCL-18, CCL-19, CCL-20, CCL-21, CCL-22, CCL-23, CCL-24, CCL-25, CCL-26, CCL-27, CCL-28, CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, CXCL17, XCL1, XCL2, fractalkine, IFN-α, IFN-β, IFN-γ, IFN-ε, IFN-κ, IFN-ω, IL10R2, IFNLR1, CD40LG, CD70, EDA, FASLG (TNFSF6), LTA (TNFSF1), LTB (TNFSF3), TNFα, TNFSF4 (OX40L), TNFSF8 (CD153), TNFSF9, TNFSF10 (TRAIL), TNFSF11 (RANKL), TNFSF12 (TWEAK), TNFSF13, TNFSF13B, TNFSF14, TNFSF15, TNFSF18, CSF1, CSF2 (GM-CSF), and CSF3 (G-CSF). More preferred examples of cytokines include IL-2, IL6, IL-10, IL-12, IL-15, IL-17, interferons, GM-CSF, and TNF. Cytokines are produced by a broad range of cells, including immune cells like macrophages, B lymphocytes, T lymphocytes and mast cells, as well as endothelial cells, fibroblasts, and various stromal cells, whereby a given cytokine may be produced by more than one type of cell. An antibody or antibody fragment comprising a functional domain comprising such a cytokine or a functional fragment thereof may elicit a pro-inflammatory immunostimulating response or an anti-inflammaotry immunosuppressive or cytotoxic response, depending on the cytokine selected.

Other preferred ligands include, for example, hormones, which are peptides, polypeptides or proteins. Hormones are signaling molecules, which are transported by the circulatory system to target distant organs, in particular to regulate physiology and behaviour. Hormones are typically produced by glands in multicellular organisms. A particularly preferred hormone is (human) growth hormone. Further examples of hormones include TRH, vasopressin, insulin, prolactin, ACTH, oxytocin, atrial-natriuretic peptide (ANP), glucagon, somatostatin, cholecystokinin, gastrin, leptin, angiotensin II, basic fibroblast growth factor-2, and parathyroid hormone-related protein.

A functional fragment of a ligand may be any fragment of a ligand, which has the ability to mediate a functionality. Usually, such fragments are referred to as “domains”. Accordingly, the functional fragment of a ligand may be any domain of the ligand. Preferred examples include functional fragments (e.g., domains) of the (exemplified) ligands described above. Preferably, the functional fragment of the ligand, which is comprised in the functional domain, is an Ig-like domain.

Preferably, the functional domain (comprised in the (poly)peptide of interest) comprises or consists of an (independent) binding site. Accordingly, it is preferred that the (poly)peptide of interest comprises or consists of an (independent) binding site.

In general, the “(independent) binding site” is a region of a (poly)peptide chain to which a specific target (e.g., a molecule and/or an ion) can bind to, in particular by forming a chemical bond, for example a non-covalent bond. A non-covalent bond is a relatively weak chemical bond that does not involve an intimate sharing of electrons. Multiple noncovalent bonds often stabilize the conformation of macromolecules and mediate highly specific interactions between molecules. Accordingly, the binding site is a functional domain, which provides binding functionality. In particular, the binding site is not a linker, such as a GS-linker. A linker does typically not provide a binding functionality. Even though the binding site may optionally comprise a linker (peptide), such as a GS-linker, it does preferably not consist of a linker (peptide), such as a GS-linker. In other words, even if the binding site comprises a linker (peptide), such as a GS-linker, it preferably comprises an additional amino acid sequence mediating a function distinct from (purely) linking two peptides to each other. Accordingly, the binding site is preferably distinct from a linker (peptide), such as a GS-linker.

Preferably, the (independent) binding site is selected from the group consisting of receptors and functional fragments thereof, ligands and functional fragments thereof, CD molecules and functional fragments thereof, single chain antibodies and antigen binding fragments thereof, antigens and functional fragments thereof, and tags.

More preferably, the (independent) binding site comprises or consists of a receptor or a functional fragment thereof. Receptors are typically able to bind to a (specific) ligand. Accordingly, receptors may be also referred to as (independent) binding sites. Various receptors are described above and preferred embodiments and examples thereof apply accordingly.

In the context of the binding site, a functional fragment of a receptor is such a fragment of the receptor, which retains the receptor's ability to bind to its ligand. Since the binding site may comprise a receptor or a functional fragment thereof, it is the binding function of the receptor, to which the term “functional” refers to in the context of the binding site. Other fragments/domains of the receptor may be preferably not comprised by the (independent) binding site. For example, a receptor may comprise one or more transmembrane domain(s), which are usually not involved in the receptor's binding function, and which are, thus, preferably not included in the (independent) binding site. Accordingly, it is most preferred that the fragment of the receptor, which is comprised by the (independent) binding site, is merely the receptor's binding site (in particular without any further domains of the receptor).

It is also more preferred that, the (independent) binding site comprises or consists of a ligand or a functional fragment thereof. Ligands are typically able to bind to a (specific) receptor. Accordingly, ligands may be also referred to as (independent) binding sites. Various ligands are described above and preferred embodiments and examples thereof apply accordingly.

In the context of the binding site, a functional fragment of a ligand is such a fragment of the ligand, which retains the ligand's binding ability. Since the binding site may comprise a ligand or a functional fragment thereof, it is the binding function of the ligand, to which the term “functional” refers to in the context of the binding site. Other fragments/domains of the ligand may be preferably not comprised by the (independent) binding site. Accordingly, it is most preferred that the fragment of the ligand, which is comprised by the (independent) binding site, is merely the ligand's binding site (in particular without any further domains of the ligand).

Preferably, the (independent) binding site is a CD (cluster of differentiation) molecule or a functional fragment thereof. A CD (cluster of differentiation) molecule is a cell surface marker. CD molecules often act as receptors or ligands or are involved in cell adhesion. The CD nomenclature was developed and is maintained through the HLDA (Human Leukocyte Differentiation Antigens) workshop started in 1982. Examples of CD molecules, which may serve as binding sites in the context of the present invention, may be retrieved, for example, from a variety of sources known to the person skilled in the art, such as http://www.ebioscience.com/resources/human-cd-chart.htm, BD Bioscience's “Human and Mouse C D Marker Handbook” (retrievable at https://www.bdbiosciences.com/documents/cd_marker_handbook.pdf) or from www.hcdm.org. Accordingly, the (independent) binding site may be a CD marker, or a functional fragment thereof, for example a (human) CD marker described in the BD Bioscience's “Human and Mouse C D Marker Handbook” (retrievable at https://www.bdbiosciences.com/documents/cd_marker_handbook.pdf) or in other sources of “CD marker charts”, which typically also indicate the binding partners, such that an appropriate binding site can be selected.

A functional fragment of a CD molecule is such a fragment of the CD molecule, which retains the CD molecule's binding ability. In the context of the present invention, the binding site may comprise a CD molecule or a functional fragment thereof, and, accordingly, it is the binding function of the CD molecule to which the term “functional” refers to. Other fragments/domains of the CD molecule may be preferably not comprised by the (independent) binding site. Accordingly, it is most preferred that the fragment of the CD molecule, which is comprised by the (independent) binding site, is merely the CD molecule's binding site (in particular without any further domains of the CD molecule). Preferably, the functional fragment of the CD molecule, which is comprised by the (independent) binding site is an Ig-like domain.

Preferably, the (independent) binding site is a single chain antibody (such as scFv or VHH) or an antigen binding fragment thereof. It is also preferred that the (independent) binding site is an antigen or a functional fragment thereof, such as an epitope.

Preferably, the (independent) binding site is a single chain antibody or an antigen binding fragment thereof. A single chain antibody is a recombinant antibody consisting of one single polypeptide chain only. Preferred examples of single chain antibodies include single chain antibodies without constant domains, such as single domain antibodies, single chain antibodies based on single chain variable fragments (scFv's) and single chain diabodies (scDb), and single chain antibodies with constant domains, such as single chain Fab fragments (scFab; Hust M, Jostock T, Menzel C, Voedisch B, Mohr A, Brenneis M, Kirsch M I, Meier D, Dübel S. Single chain Fab (scFab) fragment. BMC Biotechnol. 2007 Mar. 8; 7:14).

Preferred examples of single chain antibodies based on single chain variable fragments (scFv's) include scFv (one single V_{H} and one single VL domain) and tandem scFv's, such as tandem-di-scFv (BiTE), tandem-tri-scFv and tandem-tetra-scFv.

A single domain antibody (also referred to as “nanobody”) is an antibody fragment comprising/consisting of one single (monomeric) variable domain only. Like a whole antibody, a single domain antibody is able to bind selectively to a specific antigen. The first single domain antibodies were engineered from heavy-chain antibodies found in camelids; these are called “VHH” or “VHH fragments”. Cartilaginous fishes also have heavy-chain antibodies (IgNAR, ‘immunoglobulin new antigen receptor’), from which single-domain antibodies, called “V_{NAR}” or “V_{NAR} fragments”, can be obtained. An alternative approach is to split the dimeric variable domains from common immunoglobulin G (IgG) from humans or mice into monomers. Accordingly, single domain antibodies may be derived from heavy or light chain variable domains (V_{H} or VL). Preferred examples of single domain antibodies include VHH, VNAR, IgG-derived V_{H} and IgG-derived VL.

Most preferably, the functional domain is a VHH or an scFv. A most preferred example of a VHH is T3-VHH or F4-VHH. For example, the single domain antibody preferably comprises or consists of an amino acid sequence as set forth in SEQ ID NO: 91 or 93 or a sequence variant thereof having at least 70%, preferably at least 75%, more preferably at least 80%, even more preferably at least 85%, still more preferably 90%, particularly preferably 95%, and most preferably at least 98% sequence identity. A most preferred example of an scFv is TT39.7-scFv or MPE8-scFv. For example, the single domain antibody preferably comprises or consists of an amino acid sequence as set forth in SEQ ID NO: 92 or 94 or a sequence variant thereof having at least 70%, preferably at least 75%, more preferably at least 80%, even more preferably at least 85%, still more preferably 90%, particularly preferably 95%, and most preferably at least 98% sequence identity.

T3-VHH:
[SEQ ID NO: 91]
MAQVQLVESGGGLVQAGGSLTLSCAASGSTSRSYALGWFRQAPGKEREFV
AHVGQTAEFAQGRFTISRDFAKNTVSLQMNDLKSDDTAIYYCVASNRGWS
PSRVSYWGQGTQVTVSS
TT39.7-scFv:
[SEQ ID NO: 92]
QITLKESGPTLVKPTQTLTLTCTFSGFSLSTSRVGVGWIRQPPGKALEWL
SLIYWDDEKHYSPSLKNRVTISKDSSKNQVVLTLTDMDPVDTGTYYCAHR
GVDTSGWGFDYWGQGALVTVSSGGGGSGGGGSGGGGSQSALTQPASVSGS
PGQSITISCSGAGSDVGGHNFVSWYQQYPGKAPKLMIYDVKNRPSGVSYR
FSGSKSGYTASLTISGLQAEDEATYFCSSYSSSSTLIIFGGGTRLTVL
F4-VHH:
[SEQ ID NO: 93]
QVQLQESGGGLVQPGGSLRLSCAASGFTLDYYYIGWFRQAPGKEREAVSC
ISGSSGSTYYPDSVKGRFTISRDNAKNTVYLQMNSLKPEDTAVYYCATIR
SSSWGGCVHYGMDYWGKGTQVTVSS
MPE8-scFv:
[SEQ ID NO: 94]
EVQLVESGGGLVKPGGSLRLSCAASGFTFSSYSMNWVRQAPGKGLEWVSS
ISASSSYSDYADSAKGRFTISRDNAKTSLFLQMNSLRAEDTAIYFCARAR
ATGYSSITPYFDIWGQGTLVTVSSGGGGSGGGGSGGGGSQSVVTQPPSVS
GAPGQRVTISCTGSSSNIGAGYDVHWYQQLPGTAPKLLIYDNNNRPSGVP
DRFSASKSGTSASLAITGLQAEDEADYYCQSYDRNLSGVFGTGTKVTVL

Preferably, the (independent) binding site is an antigen or a functional fragment thereof, in particular an epitope. An antigen is a molecule, or a portion of a molecule capable of being bound by an antibody. As the antigen, or the functional fragment thereof, is comprised by the polypeptide chain, it is understood that in the context of the present invention, if the binding site is an antigen or a functional fragment thereof, said antigen or functional fragment thereof is a peptide or polypeptide. An antigen typically comprises one or more epitopes. The epitope is that part of the antigen, which is bound by an antibody (“recognized” by an antibody).

Preferred examples of antigens include, but are not limited to, serum proteins, e.g. cytokines such as IL4, IL5, IL9 and IL13, bioactive peptides, cell surface molecules, e.g. receptors, transporters, ion-channels, viral and bacterial proteins, RAGE (Receptor for Advanced Glycosylation End Products), GPVI and collagen.

A functional fragment of an antigen is such a fragment of the antigen, which retains the antigen's binding ability. Accordingly, the fragment of the antigen is preferably an epitope or it comprises one or more epitopes. Other fragments/domains of the antigen may be preferably not comprised by the (independent) binding site. Accordingly, it is most preferred that the fragment of the antigen, which is comprised by the (independent) binding site, is an epitope or includes more than one epitope (in particular without any further domains of the antigen).

It is also preferred that the (independent) binding site is a tag comprising a binding site. Most tags are able to bind, e.g. affinity tags. Accordingly, those tags, which have the ability to bind to another molecule, may be also referred to as (independent) binding sites. Various tags, including tags comprising a binding site, are described above and preferred embodiments and examples apply accordingly.

Most preferably, the functional domain is an Ig-like domain, an scFv, a VHH or a Strep-tag. In particular, the functional domain preferably comprises or consists of an amino acid sequence as set forth in any of SEQ ID NOs 65, 79, and 88-94, or a sequence variant thereof having at least 80%, preferably at least 85%, more preferably at least 90%, even more preferably at least 95% and most preferably at least 98% sequence identity.

In a particularly preferred embodiment, the (poly)peptide of interest comprises or consists of a V_{H} domain or a V_{H}-V_{L} domain, as described above. It is also preferred that the (poly)peptide of interest comprises or consists of a pathogen binding domain, i.e. a domain comprising or consisting of a binding site, which is capable of specifically binding to a pathogen. In general, the term “pathogen” refers to anything that can produce disease, in particular an infectious agent derived from a micro-organism or micro-organism itself. The pathogen may be selected from a bacterial pathogen, a viral pathogen, a fungal pathogen, a prionic pathogen, a protozoon pathogen, a pathogen of (another) (human) parasite, e.g. helminths, or an algal pathogen.

A (poly)peptide of interest comprising or consisting of CD4, dipeptidyl peptidase 4, CD9, or angiotensin-converting enzyme 2 or a fragment or sequence variant thereof is particularly preferred. For example, cluster of differentiation (CD) 4 binds human immunodeficiency virus (HIV). For example, dipeptidyl peptidase 4 (DPP-4) and CD9 are targeted by the Middle East Respiratory Syndrom Coronavirus” (MERS-CoV). For example, angiotensin-converting enzyme 2 (ACE2) binds to severe acute respiratory syndrome (SARS-CoV).

A particularly preferred example of the nucleotide sequence encoding a (poly)peptide of interest comprises or consists of a nucleotide sequence as set forth in SEQ ID NO: 111; or a (functional) sequence variant thereof having at least 70%, at least 75%, preferably at least 80%, preferably at least 85%, more preferably at least 88%, more preferably at least 90%, even more preferably at least 92%, even more preferably at least 95%, still more preferably at least 96%, still more preferably at least 97%, and most preferably at least 98% or at least 99% sequence identity.

Accordingly, particularly preferred examples of the DNA molecule to be introduced into the isolated B cell according to the present invention include a DNA molecule comprising or consisting of a nucleotide sequence as set forth in SEQ ID NO: 99 or 110; or a (functional) sequence variant thereof having at least 70%, at least 75%, preferably at least 80%, preferably at least 85%, more preferably at least 88%, more preferably at least 90%, even more preferably at least 92%, even more preferably at least 95%, still more preferably at least 96%, still more preferably at least 97%, and most preferably at least 98% or at least 99% sequence identity.

B Lymphocyte Culture and Activation of AID

As described above, the present invention provides a method for editing the genome of a B lymphocyte. In particular, the genome of B lymphocytes may be edited to such that the B lymphocyte does not express its endogenous (i.e, naturally recombined) B cell receptor (BCR), as described above. For example, the genome of the B lymphocyte may be edited to substitute the endogenous B cell receptors (BCRs) with sequences of customized (monoclonal) antibodies.

For editing the genome of a B lymphocyte, the isolated (preferably primary) B lymphocyte is in particular provided in culture. Methods for culturing isolated (preferably primary) B cells are known in the art.

In general, the culture conditions typically comprise a “complete culture medium”. The term “culture medium” in general as used herein refers to a liquid or gel designed to support the growth of cells. A “complete culture medium” refers to a basal medium, preferably a basal synthetic medium, supplemented with at least one additional component. Non-limiting examples of complete culture media are described in WO 03/076601, WO 05/007840, EP 787180, U.S. Pat. Nos. 6,114,168, 5,340,740, 6,656,479, 5,830,510 and in Pain et al. (1996, Development 122:2339-2348).

As used herein, “basal medium” refers to a medium that allows, by itself, at least cell survival, and preferably, cell growth. In particular a basal medium has a classical medium formulation. Non-limiting examples of basal media include BME (Eagle's Basal Medium), MEM (minimum Eagle Medium), medium 199, DMEM (Dulbecco's modified Eagle Medium), Knockout DMEM, GMEM (Glasgow modified Eagle medium), DMEM-HamF12, Ham-F12 and Ham-F10, Iscove's Modified Dulbecco's medium (IMDM), MacCoy's 5A medium, and RPMI 1640. In particular, basal media comprise one or more inorganic salts (for example CaCl₂, KCl, NaCl, NaHCO₃, NaH₂PO₄, MgSO₄, etc.), one or more amino-acids, one or more vitamins (for example thiamine, riboflavin, folic acid, D-Ca pantothenate, etc.) and/or one or more other components such as for example glucose, beta-mercapto-ethanol, and sodium pyruvate.

Preferably, the basal medium is a synthetic medium. Most preferably, the basal medium is IMDM and/or RPMI.

Preferably, the culture medium of the invention further comprises animal serum, in particular fetal animal serum. The preferred animal serum is fetal bovine serum (FBS). A particular preferred FBS is HyClone (GE Healthcare Life Sciences; e.g. HyClone 40 mm filtered, SH30070.03). However, animal serum comprising serum from other animal species may also be used. The final concentration of animal serum in the culture medium is preferably approximately 0.01-10%, preferably 0.05-5%, more preferably, 0.1-2.5%, even more preferably 0.5-1.5% and most preferably about 1%.

The culture medium of the invention may preferably comprise in addition antibiotics, such as for example penicillin and streptomycin and/or kanamycin, in particular to prevent bacterial contamination. Thereby, a combination of penicillin/streptomycin is preferred. In addition, also kanamycin is preferred. For example, the finale culture medium may comprise penicillin/streptomycin and/or kanamycin. Preferably, the concentration of antibiotic in the culture medium is from 1 to 1000 U/ml, more preferably from 10 to 500 U/ml, even more preferably from 50 to 250 U/ml, and particularly preferably about 100 U/ml. For example, a combination of penicillin/streptomycin maybe used in the following concentrations of antibiotic in the final culture medium: from 1 to 1000 U/ml penicillin and from 1 to 1000 μg/ml streptomycin, more preferably from 10 to 500 U/ml penicillin and from 10 to 500 μg/ml streptomycin, even more preferably from 50 to 250 U/ml penicillin and from 50 to 250 μg/ml streptomycin, and particularly preferably about 100 U/ml penicillin and about 100 μg/ml streptomycin. It is also preferred that the concentration of penicillin/streptomycin in the final culture medium is 0.01-10%, preferably 0.05-5%, more preferably, 0.1-2.5%, even more preferably 0.5-1.5% and most preferably about 1%. It is also preferred that the concentration of kanamycin in the final culture medium is 0.01-10%, preferably 0.05-5%, more preferably, 0.1-2.5%, even more preferably 0.5-1.5% and most preferably about 1%.

In addition, culture medium of the present invention may preferably comprise further additives, e.g. a glutamine derivative, preferably GlutaMax, NEAA, a biological buffer, preferably HEPES, pyruvate (e.g. sodium pyruvate), β-mercapto-ethanol and/or transferrin. In general, the above and further additives may be used in concentrations according to the manufacturer.

The glutamine derivative may be for example L-glutamine or GlutaMax, whereby GlutaMax is preferred. GlutaMax is an L-alanyl-L-glutamine dipeptide, which is available for example as 200 mM L-alanyl-L-glutamine dipeptide in 0.85% NaCl (100× stock solution). The glutamine derivative is preferably used in the final culture medium in a concentration from 0.1 to 100 mM, more preferably from 0.5 to 50 mM, even more preferably from 1 to 10 mM, particularly preferably about 2 mM. It is also preferred that the concentration of GlutaMax in the final culture medium is 0.01-10%, preferably 0.05-5%, more preferably, 0.1-2.5%, even more preferably 0.5-1.5% and most preferably about 1%.

NEAA, i.e. non-essential-amino-acids solution, is a commercially available sterile-filtered and cell culture-tested liquid formulation with Earle's Salts Base, non-essential amino acids, sodium bicarbonate (NaHCO₃), and phenol red as pH indicator, but without L-Glutamine. The concentration of NEAA in the final culture medium is usually according to the manufacturer, e.g. 1:100 in case of a 100× stock solution. It is also preferred that the concentration of NEAA in the final culture medium is 0.01-10%, preferably 0.05-5%, more preferably, 0.1-2.5%, even more preferably 0.5-1.5% and most preferably about 1%.

Pyruvate is an intermediary organic acid metabolite in glycolysis and the first of the Embden Myerhoff pathway that can pass readily into or out of the cell. Thus, its addition to a cell culture medium provides both an energy source and a carbon skeleton for anabolic processes. A preferred pyruvate is sodium pyruvate, which may also help to reduce fluorescent light-induced phototoxicity. Pyruvate, preferably sodium pyruvate, is preferably used in the culture medium in a concentration from 0.05 to 50 mM, more preferably from 0.1 to 10 mM, even more preferably from 0.5 to 5 mM, particularly preferably about 1 mM. It is also preferred that the concentration of pyruvate, preferably sodium pyruvate, in the final culture medium is 0.01-10%, preferably 0.05-5%, more preferably, 0.1-2.5%, even more preferably 0.5-1.5% and most preferably about 1%.

Beta-mercapto-ethanol (also referred to as 2-Mercaptoethanol, β-ME or 2-ME) is assumed to act as a free radical scavenger. Beta-mercapto-ethanol is preferably used in the final culture medium in a concentration from 0.005 to 5.0 mM, more preferably from 0.01 to 1.0 mM, even more preferably from 0.05 to 0.5 mM, particularly preferably about 0.1 mM. It is also preferred that the concentration of beta-mercapto-ethanol in the final culture medium is 0.01-10%, preferably 0.05-5%, more preferably, 0.1-2.5%, even more preferably 0.5-1.5% and most preferably about 1%.

Moreover, it is also preferred that the concentration of transferrin in the final culture medium is 0.01-10%, preferably 0.05-5%, more preferably, 0.1-2.5%, even more preferably 0.5-1.5% and most preferably about 1%.

Thus, a particularly preferred culture medium according to the present invention comprises:
a basal medium, preferably RPMI or IMDM;
preferably an animal serum, more preferably FBS;
preferably an antibiotic, more preferably penicillin/streptomycin and/or kanamycin; and
preferably further additives, including for example a glutamine derivative (preferably GlutaMax), NEAA, pyruvate (e.g. sodium pyruvate), β-mercapto-ethanol, and/or transferrin.

Most preferably, B lymphoctes are cultured in RPM) or IMDM with 10% FBS, 1% NEAA, 1% sodium pyruvate, 1% beta-mercaptoethanol, 1% Glutamax, 1% penicillin/streptomycin, 1% kanamycin, and 1% transferrin.

In another example, B lymphocytes may be cultured in RPM) (e.g., RPMI-1640) with 10% FBS, 1% P/S (penicillin/streptomycin), 1% HEPES (4-(2-hydroxyethyl)-1-piperazineethanesulfonic acid), and 1% L-glutamine.

Preferably, B lymphocytes are cultured at a density of about or between 0.5×10⁵ to 10×10⁶ cells/ml, preferably at 1×10⁵ to 1×10⁶ cells/ml, more preferably at 1×10⁵ to 5×10⁵ cells/ml, even more preferably at 1.5×10⁵ to 2.5×10⁵ cells/ml, and most preferably at about 2×10⁵ cells/ml.

In step (i) of the method according to the present invention, the B cell's endogenous AID is activated. Preferably, activation of activation-induced cytidine deaminase of the B lymphocyte may be achieved, for example, by culturing B cells in a culture medium comprising an activator of activation-induced cytidine deaminase. Preferably, the activator of activation-induced cytidine deaminase is selected from the group consisting of: a cytokine, an anti-B cell receptor antibody or fragments thereof, a TLR agonist, a CpG-B agonist, an imidazoquinoline compound or a combination of any of said activators. In other words, it is preferred that the B lymphocyte is cultured in a culture medium comprising a cytokine, an anti-B cell receptor antibody or fragments thereof, a TLR agonist, a CpG-B agonist and/or an imidazoquinoline compound.

For activation of the B cell's endogenous AID the B lymphocyte is preferably cultured in a cell culture medium comprising an activator of activation-induced cytidine deaminase (such as a cytokine, an anti-B cell receptor antibody or fragments thereof, a TLR agonist, a CpG-B agonist and/or an imidazoquinoline compound) from about 3 hours to 10 days, preferably from about 6 hours to 7 days, more preferably from about 12 hours to 5 days, even more preferably from about 18 hours to 3 days, still more preferably from about 21 hours to 2 days.

Most preferably, the B cells are cultured in a cell culture medium comprising an activator of activation-induced cytidine deaminase (such as a cytokine, an anti-B cell receptor antibody or fragments thereof, a TLR agonist, a CpG-B agonist and/or an imidazoquinoline compound) for about 24 hours.

Accordingly, it is preferred that the B lymphocyte is cultured in a cell culture medium comprising an activator of activation-induced cytidine deaminase (such as a cytokine, an anti-B cell receptor antibody or fragments thereof, a TLR agonist, a CpG-B agonist and/or an imidazoquinoline compound) for about 3 hours to 10 days prior to introduction of the DNA molecule (step (ii)), preferably about 6 hours to 7 days prior to introduction of the DNA molecule (step (ii)), more preferably about 12 hours to 5 days prior to introduction of the DNA molecule (step (ii)), even more preferably about 18 hours to 3 days prior to introduction of the DNA molecule (step (ii)), still more preferably about 21 hours to 2 days prior to introduction of the DNA molecule (step (ii)), and most preferably for about 24 hours prior to introduction of the DNA molecule (step (ii)).

Moreover, it is preferred that the B lymphocyte is cultured in a cell culture medium comprising an activator of activation-induced cytidine deaminase (such as a cytokine, an anti-B cell receptor antibody or fragments thereof, a TLR agonist, a CpG-B agonist and/or an imidazoquinoline compound) for at least 3 hours prior to introduction of the DNA molecule (step (ii)), preferably at least 6 hours prior to introduction of the DNA molecule (step (ii)), more preferably at least 12 hours prior to introduction of the DNA molecule (step (ii)), even more preferably at least 18 hours prior to introduction of the DNA molecule (step (ii)), and most preferably at least 21 hours prior to introduction of the DNA molecule (step (ii)), such as about 24 hours prior to introduction of the DNA molecule (step (ii)).

It is also preferred that the B lymphocyte is cultured in a cell culture medium comprising an activator of activation-induced cytidine deaminase (such as a cytokine, an anti-B cell receptor antibody or fragments thereof, a TLR agonist, a CpG-B agonist and/or an imidazoquinoline compound) for no more than 10 days prior to introduction of the DNA molecule (step (ii)), preferably no more than 7 days prior to introduction of the DNA molecule (step (ii)), more preferably no more than 5 days prior to introduction of the DNA molecule (step (ii)), even more preferably no more than 3 days prior to introduction of the DNA molecule (step (ii)), still more preferably no more than 2 days prior to introduction of the DNA molecule (step (ii)), and most preferably for no more than about 36 hours prior to introduction of the DNA molecule (step (ii)), such as about 24 hours prior to introduction of the DNA molecule (step (ii)).

In other words, in the method according to the present invention it is preferred that introducing the DNA molecule into the B lymphocyte is performed up to 10 days after activating the activation-induced cytidine deaminase, preferably up to 7 days after activating the activation-induced cytidine deaminase, more preferably up to 5 days after activating the activation-induced cytidine deaminase, even more preferably up to 2 days after activating the activation-induced cytidine deaminase and most preferably about 1 day after activating the activation-induced cytidine deaminase.

As described above, the activator of activation-induced cytidine deaminase is preferably a cytokine, an anti-B cell receptor antibody or fragments thereof, a TLR agonist, a CpG-B agonist and/or an imidazoquinoline compound.

Preferred the Toll-like receptor (TLR) agonist is an agonist of TLR7 or TLR9, such as R848 or CL264. Preferred examples of anti-B cell receptor antibody or fragments thereof include anti B cell receptor F(ab′)2-fragments specific for human immunoglobulins. A preferred example of a CpG-B agonist is ODN2006. A preferred example of an imidazoquinoline compound is Clo97.

Examples of cytokines include IL1-like, IL1a, IL1β, IL1RA, IL18, CD132, IL2, IL4, IL7, IL9, IL13, IL15, CD131, IL3, IL5, GM-CSF, IL6-like, IL6, IL11, G-CSF, IL12, LIF, OSM, IL10, IL20, IL21, IL14, IL16, IL17, IFNα, IFNβ, IFNγ, CD154, LT13, TNFα, TNF13, 4-1BBL, APRIL, BAFF, CD70, CD153, CD178, CD30L, CD40L, GITRL, LIGHT, OX40L, TALL-1, TRAIL, TWEAK, TRANCE, TGFβI, TGFβ2, TGFβ3, c-Kit, FLT-3, Epo, Tpo, FU-3L, SCF, M-CSF, aCD40, or any combinations thereof. Preferably the cytokine is selected from the group consisting of CD40L, IL4, IL2, IL21, BAFF, APRIL, CD30L, TGF-B1, 4-1BBL, IL6, IL7, IL10, IL13, c-Kit, FLT-3, IFNα, or any combination thereof. Most preferably, the B lymphocyte is cultured in a medium comprising IL4 and/or CD40L.

For example, the B cells may be co-cultured with a CD40L expressing cell line (e.g. K562L or 3T3 cells) prior to introduction of the DNA molecule (step (ii); transfection). The B cells may be co-cultured for at least 12, 24, 36, 48, or 72 hours prior to transfection.

Preferably, the concentration of the cytokine (in the culture medium) is 0.001-20 ng/ml, preferably 0.001-1 ng/ml, more preferably 0.005-0.5 ng/ml, even more preferably 0.01-0.1 ng/ml, still more preferably 0.015-0.02 ng/ml and most preferably about 0.16 ng/ml.

Moreover, it is preferred that the B lymphocyte is cultured in a cell culture medium comprising a cytokine at a concentration of at least 0.001 ng/ml, preferably at least 0.001 ng/ml, more preferably at least 0.005 ng/ml, even more preferably at least 0.01 ng/ml, still more preferably at least 0.015 ng/ml and most preferably about 0.16 ng/ml.

It is also preferred that the B lymphocyte is cultured in a cell culture medium comprising a cytokine at a concentration of no more than 20 ng/ml, preferably no more than 1 ng/ml, more preferably no more than 0.5 ng/ml, even more preferably no more than 0.1 ng/ml, still more preferably no more than 0.02 ng/ml and most preferably about 0.16 ng/ml.

In a preferred embodiment activating the activation-induced cytidine deaminase is performed by (cultivation of the B lymphocyte in presence of) CD40L and/or IL4. In other words, it is preferred that the B lymphocyte is cultured in a cell culture medium comprising CD40L and/or IL4. More preferably, activating of the activation-induced cytidine deaminase is performed by co-culture with a CD40L expressing cell line (as described above) and addition of IL-4 (to the culture medium as described above). Most preferably, the the CD40L expressing cell line is K562L.

In general, the concentration of IL4 in the culture medium may be as described above in general for cytokines. In particular, the concentration of IL4 (in the final culture medium) is preferably 0.005-0.03 ng/ml, more preferably 0.01-0.025 ng/ml, even more preferably 0.015-0.02 ng/ml and most preferably 0.16 ng/ml.

Preferably, the B lymphocytes are reactivated after introducing the DNA molecule into the B lymphocyte (post transfection). Reactivation of cells post-transfection improves viability. For reactivation, the B cell stimulating agents as described above may be used, namely, a cytokine, an anti-B cell receptor antibody or fragments thereof, a TLR agonist, a CpG-B agonist and/or an imidazoquinoline compound as described above. In other words, it is preferred that B cell stimulating agents, for example as defined above (a cytokine, an anti-B cell receptor antibody or fragments thereof, a TLR agonist, a CpG-B agonist and/or an imidazoquinoline compound), are applied to the B lymphocyte after introducing the DNA molecule into the B lymphocyte.

In general, the B lymphocyte may be reactivated once or repeatedly after introducing the DNA molecule into the B lymphocyte (post transfection). For example, B lymphocytes may be reactivated for about 1, 2, 3, 4, 5, or more days. Preferably, B cells are reactivated (for the first time after transfection) no later than 24 hours post transfection, for example about 1, 2, 3, 4, 5, 6, 7, 8, 9, 10, 11, 12, 13, 14, 15, 16, 17, 18, 19, 20, 21, 22, 23, or 24 hours post transfection. More preferably B cells are reactivated no later than 18 hours post transfection, even more preferably B cells are reactivated no later than 12 hours post transfection, and most preferably B cells are reactivated no later than 6 hours post transfection, such as 3-5 hours post transfection, for example about 4 hours post transfection. Such reactivation is most preferably performed with IL4. Moreover, reactivation (e.g. with IL4) is preferably repeated every 1-5 days, preferably every 2-4 days, most preferably every 3 days.

In addition or alternatively, B lymphocytes are particularly preferably reactivated by CD40L expressing cells, such as K562L cells, for example once or repeatedly, most preferably once at 2 to 10 days post transfection, preferably 4 to 9 days post transfection, more preferably 6 to 8 days post transfection, such as about 7 days post transfection.

In a particularly preferred embodiment, B cells are reactivated 4 h post transfection and in consecutive intervals of 3 days with IL4 and on day 7 with K562L cells.

Preferably, the B lymphocyte is treated with a DNA inhibitor capable of blocking alternative-end joining before introducing the DNA molecule into the B lymphocyte. A preferred example of such a DNA inhibitor is Olaparib. Such pretreatment blocks the alternative-end joining (a-EJ) pathway, thereby enforcing the use of the c-NHEJ pathway and further increasing engineering efficiencies.

It is also preferred that the DNA molecule comprising a nucleotide sequence encoding the (poly)peptide of interest is incubated with a Ku protein, such as Ku70/Ku80, before introducing the DNA molecule into the B lymphocyte. Accordingly, in a preferred embodiment a DNA molecule/Ku protein complex (formed during incubation) is introduced into the B lymphocyte. Thereby, the number of successful integrations of the DNA molecule may be further increased.

It is also preferred that the DNA molecule comprises a nuclear localization signal, such as SV40 nuclear localization signal, for example the SV tandem repeat according to SEQ ID NO: 95, or a sequence variant thereof:

SV Tandem Repeat:

[SEQ ID NO: 95]
TGGTTGCTGACTAATTGAGATGCATGCTTTGCATACTTCTGCCTGCTGGG
GAGCCTGGGGACTTTCCACACCTGGTTGCTGACTAATTGAGATGCATGCT
TTGCATACTTCTGCCTGCTGGGGAGCCTGGGGACTTTCCACACC

Thereby, high concentrations of the DNA molecule in the nucleus of the B lymphocyte may be achieved, which in turn also further increase integration of the DNA molecule into the genome of the B lymphocyte.

Moreover, it is also preferred to treat the B lymphocyte with a nuclease inhibitor, in particular at least about 24 hours after activation of the activation-induced cytidine deaminase of the B lymphocyte. A preferred example of a nuclease inhibitor is Mirin. Thereby, degradation of the DNA molecule introduced into the B cell by the B cell's endo- and/or exonucleases can be avoided.

Transfection

As described above, methods for introducing the DNA molecule comprising a nucleotide sequence encoding the (poly)peptide of interest (i.e. methods of transfection) encompass, for example, viral and non-viral methods of transfection. Viruses which may be used for gene transfer include retrovirus (including lentivirus), herpes simplex virus, adenovirus and adeno-associated virus (AAV). However, in some embodiments the B lymphocyte is not transduced with a retrovirus. Moreover, nanoparticles may also be used for transfection. Further non-viral transfection methods include many chemical and physical methods. Chemical transfection methods include lipofection, e.g. based on cationic lipids and/or liposomes, calcium phosphate precipitation, or transfection based on cationic polymers, such as DEAE-dextran or polyethylenimine (PEI) etc. Physical transfection methods include electroporation, ballistic gene transfer (introduces particles coated with DNA into cells), microinjection (DNA transfer through microcapillaries into cells), and nucleofection. Preferably, the introduction of the DNA molecule comprising a nucleotide sequence encoding a (poly)peptide of interest into the B lymphocyte is non-viral.

Most preferably, the DNA molecule is introduced into the B lymphocyte by nucleofection. Nucleofection is an electroporation-based transfection method which enables transfer of nucleic acids such as DNA and RNA into cells by applying a specific voltage. Based on the physical method of electroporation, nucleofection uses a combination of electrical parameters, generated by a nucleofection device (“Nucleofector”), preferably with cell-type specific reagents. The DNA molecule (substrate) is transferred directly into the cell nucleus and the cytoplasm. Accordingly, it is preferred that a nucleofection device is used for nucleofection. In general, any nucleofection device may be used, for example Neon®, MaxCyte or Amaxa®. Preferably, the Amaxa® or Neon® Nucleofector is used.

In general, any nucleofection program provided by the manufacturer of the nucleofection device may be used. Preferably, 2100-2500 V are used with 1 or 2 pulses of 10-20 ms (msec). More preferably, 2100-2400 V are used with 1 or 2 pulses of 10-20 ms (msec). For example, a single pulse of 2150V and 10 ms may be used. For example, a single pulse of 2150V and 15 ms may be used. For example, a single pulse of 2150V and 20 ms may be used. For example, two pulses of 2150V and 10 ins may be used. For example, a single pulse of 2400V and 10 ms may be used. For example, a single pulse of 2400V and 15 ms may be used. For example, a single pulse of 2400V and 20 ms may be used. For example, a single pulse of 2500V and 10 ms may be used. For example, a single pulse of 2500V and 15 ms may be used. Most preferably, (exactly) two pulses of 2150V and 10 ms are used, in particular with the Neon® Nucleofector.

Preferably, a nucleofector kit for B cells (e.g., Lonza's Nucleofector kit for human B cells) is used, in particular in combination with the Amaxa® Nucleofector, for example according to the manufacturer's instructions. Most preferably, the Amaxa® Nucleofector is used in combination with Lonza's Nucleofector kit for human B cells as described in manufacturer's instructions with U15 program, wherein preferably the cell number is (changed to) 2 million and/or the amount of DNA is (changed to) about 2.5 μg per nucleofection for the Amaxa® Nucleofector.

Preferably, the DNA is transfected at an amount (amount per transfection, in particular nucelofection) of about and between 0.5 μg to 10 μg of DNA, for example the DNA concentration may be 1, 2, 3, 4, 5, 6, 7, 8, 9, or 10 μg. More preferably, the DNA concentration is transfected at an amount (amount per transfection, in particular nucelofection) of about and between 1 μg to 5 μg DNA, even more preferably 1.5 μg to 3.5 μg DNA, still more preferably 1.0 to 3.0 μg DNA, and most preferably the amount of DNA per transfection is about 2.5 μg DNA.

Preferably, the genome-edited B cells are used directly after the genome editing process or after a short culture period. For clinical use, the genome-edited B cells may be irradiated prior to use. Irradiation induces expression of cytokines, which promote immune effector cell activity.

Engineered B Lymphocytes and Uses Thereof

In a further aspect, the present invention also provides an engineered B lymphocyte obtainable by the method according to the present invention as described herein. In other words, the present invention also provides an engineered B lymphocyte made by the method according to the present invention as described herein.

Accordingly, it is understood that the detailed description and preferred embodiments of the method for editing the genome of a B lymphocyte according to the present invention outlined above apply accordingly to the engineered B lymphocyte obtainable by such a method. For example, the detailed description of edited B cells described above and, in particular, preferred (poly)peptides of interest apply accordingly to the B cell obtainable by the inventive method.

In general B cells obtained by the inventive method can be easily recognized due to the heterologous insert in the switch region of the B cell genome.

Accordingly, the present invention also provides an engineered B lymphocyte comprising an edited immunoglobulin gene locus comprising a heterologous insert comprising a nucleotide sequence encoding a (poly)peptide of interest inserted in its switch region. The term “heterologous” refers to a sequence, which is distinct from the endogenous sequence, i.e. the sequence, which was originally at this genomic location. In general, the DNA molecule comprising a nucleotide sequence encoding a (poly)peptide of interest described above essentially corresponds to the heterologous insert. Accordingly, the detailed description and preferred embodiments of the DNA molecule comprising a nucleotide sequence encoding a (poly)peptide of interest described above apply accordingly to the heterologous insert. In particular, the (poly)peptide of interest is the same as described above.

In general, heterologous insert is inserted into the switch region of the immunoglobulin gene locus. Thereby, the immunoglobulin gene locus is edited. In general, also for the engineered B lymphocyte the detailed description and preferred embodiments of the method for editing the genome of a B lymphocyte according to the present invention outlined above apply accordingly.

In general, the engineered B lymphocytes of the invention may be of any species. In some embodiments, the engineered B lymphocyte is a mammalian B lymphocyte. Preferably, the engineered B lymphocyte according to the present invention is human. Accordingly, in some embodiments the engineered B lymphocyte is not a chicken or murine B lymphocyte. In particular, the IgL locus of the B lymphocyte is preferably not deleted.

In particular, in the engineered B cell according to the present invention the genome of the B cell is preferably edited to express a modified immunoglobulin chain comprising in N- to C-terminal direction: a variable domain, the (poly)peptide of interest (encoded by the DNA molecule introduced in step (ii)) and a constant domain. In other words, the genome of the B lymphocyte is preferably edited to express a modified immunoglobulin chain comprising the (poly)peptide of interest arranged between a variable domain and a constant domain of the immunoglobulin chain. Accordingly, it is preferred that the genome of the B lymphocyte is edited to express a modified antibody comprising the (poly)peptide of interest in the elbow region of the antibody. Moreover, it is preferred that the genome of the B lymphocyte is edited to express a modified B-cell receptor comprising the (poly)peptide of interest in the elbow region of the antibody. In this context, the detailed description outlined above, in the context of the method according to the present invention applies accordingly.

It is also preferred that the genome of the B lymphocyte is edited to express a modified immunoglobulin chain, wherein an endogenous variable domain is replaced by the (poly)peptide of interest. Accordingly, it is also preferred that the genome of the B lymphocyte is edited to express a modified B-cell receptor, wherein an endogenous variable domain is replaced by the (poly)peptide of interest. Accordingly, it is preferred that the genome of the B lymphocyte is edited to express a modified antibody comprising the (poly)peptide of interest “instead” of an endogenous variable domain. Again, the detailed description outlined above, in the context of the method according to the present invention applies accordingly.

It is also preferred that the genome of the B lymphocyte is edited to express a modified immunoglobulin chain, wherein the endogenous constant domains are replaced by the (poly)peptide of interest. Accordingly, it is also preferred that the genome of the B lymphocyte is edited to express a modified B-cell receptor, wherein the endogenous constant domains are replaced by the (poly)peptide of interest. Accordingly, it is preferred that the genome of the B lymphocyte is edited to express a modified antibody comprising the (poly)peptide of interest “instead” of the endogenous constant domains. Accordingly, such a modified immunoglobulin chain comprises an (endogenous) variable domain, the (poly)peptide of interest, but no (endogenous) constant domain. Again, the detailed description outlined above, in the context of the method according to the present invention, applies accordingly.

Preferably, the switch region of an immunoglobulin gene locus of the engineered B lymphocytes according to the present invention comprises a cleavage site, more preferably a self-processing site, such as a T2A cleavage site. Again, the detailed description outlined above, in the context of the method according to the present invention, for cleavage sites applies accordingly.

Preferably, the switch region of an immunoglobulin gene locus of the engineered B lymphocyte according to the present invention comprises a nucleotide sequence encoding a pathogen binding domain, a V_{H} domain, or a V_{H}-V_{L} domain. It is also preferred that the switch region of an immunoglobulin gene locus of the engineered B lymphocyte according to the present invention comprises a nucleotide sequence encoding a CD4, dipeptidyl peptidase 4, CD9, or angiotensin-converting enzyme 2 or a fragment or sequence variant thereof. Again, the detailed description outlined above, in the context of the method according to the present invention, applies accordingly.

In some embodiments the engineered B lymphocyte does not express GFP (green fluorescent protein) or RFP (red fluorescent protein, such as tdTomato or DsRed). In more general, in some embodiments the engineered B lymphocyte does not express a (fluorescent) reporter protein.

In general, the engineered B cells may be used for any application in which it is desired to modulate B cell receptor expression, specificity, and/or functionality. Preferably, the engineered B lymphocytes are used in medicine, i.e. for medical use, for example in immunotherapy. To this end, the B lymphocyte is preferably engineered (i.e. genome edited) as described herein.

In general, diseases to be targeted by the engineered B lymphocyte according to the present invention include any diseases, which may be treated with (monoclonal) antibodies. Such diseases include cancer, infectious diseases, autoimmune disorders, transplant rejection, osteoporosis, macular degeneration, multiple sclerosis, and cardiovascular diseases. Treatment and/or prevention of cancer and/or infectious diseases is preferred.

Preferably, the engineered B cell according to the present invention may be used for (the preparation of a medicament for) the prophylaxis, treatment and/or amelioration of cancer or tumor diseases. In general, the term “cancer” includes solid tumors, in particular malignant solid tumors, such as sarcomas, carcinomas and lymphomas, and blood cancer, such as leukemias. Cancers include carcinomas, sarcomas, lymphomas, keukemias, germ cell tumors and blastomas.

Preferably, the engineered B cells according to the present invention may be used for (the preparation of a medicament for) the prophylaxis, treatment and/or amelioration of an infectious disease. Infectious diseases include viral, retroviral, bacterial and protozoological infectious diseases.

Moreover, the engineered B cell according to the present invention may be used for (the preparation of a medicament for) the prophylaxis, treatment and/or amelioration of autoimmune disorders. Typically, autoimmune diseases arise from an abnormal immune response of the body against substances and tissues normally present in the body (autoimmunity). This may be restricted to certain organs or may involve a particular tissue in different places. Autoimmune diseases may be classified by corresponding type of hypersensitivity: type 1 (i.e. urticaria induced by autologous serum), type 11, type III, or type IV.

For medical use, the engineered B lymphocyte is preferably administered to a patient. The B lymphocyte administered to a patient may be an autologous B lymphocyte (i.e. the B cell is administered to the same patient from which the B cell or their progenitor cells were isolated prior to engineering) or an allogenic B lymphocyte (of another (human) origin, i.e. the B cell is not derived from the patient to whom it is administered after engineering). Most preferably, the B cell is an autologous B lymphocyte, i.e. the patient receiving the engineered B lymphocyte is the same patient from whom the B lymphocyte (or its progenitor cell(s)) was isolated prior to engineering.

Accordingly, the present invention also provides a method for B cell therapy comprising the following steps:
(a) isolating a (non-engineered) B lymphocyte from a subject;
(b) engineering the B lymphocyte according to the present invention as described herein; and
(c) administering the engineered B lymphocyte to the (same) subject.

If an engineered B lymphocyte (autologous or allogenic) is administered to a subject/patient, it is preferred that before administration to the subject/patient the B lymphocyte is tested (e.g., in vitro) regarding mutations known to be involved in the (development of) cancer (i.e. whether or not such mutations occur in the B cell). Examples of such cancer-causing mutations include chromosomal translocation. Thereby, engineered B lymphocytes, which were identified to carry mutations known to be involved in the (development of) cancer can be rejected, i.e. engineered B lymphocytes, which were identified to carry mutations known to be involved in the (development of) cancer are not administered to the patient. Thereby, the risk of administering a B cell with a cancer-causing mutation is strongly reduced.

Methods for testing for cancer-causing mutations in B cells are known in the art. For example, the loss of immunoglobulin on the surface of a B cell is an indicator for a cancer causing mutation. Accordingly, engineered B cells may be selected for preserved BCR surface expression before administering the B cell to the patient. Accordingly, it is preferred that before B cell administration to the patient, it is confirmed that the B cell expresses an immunoglobulin/B cell receptor on its surface.

Alternatively or additionally the engineered B cell may also be checked for the presence of particular oncogenes before administration to the patient/subject. Examples of such oncogenes include BCL6, BCL2 (MCL1), BCL11 and MALT1. Accordingly, it is preferred that before B cell administration to the patient, it is confirmed that the B cell does not show dysregulated expression (e.g., overexpression) of an oncogene, for example BCL6, BCL2 (MCL1), BCL11 and/or MALT1.

In a further aspect, the present invention also provides a cell line of engineered B lymphocytes as described herein. In particular, the term “cell line” refers to an immortalized cell line. An immortalized cell line is a population of cells from a multicellular organism which is immortalized and can therefore be grown for prolonged periods in vitro. Methods for immortalizing B cells are known in the art. Preferably, EBV (Epstein-Barr virus) immortalization is used. For example, an improved method for B cell immortalization with EBV is described in Traggiai E, Becker S, Subbarao K, Kolesnikova L, Uematsu Y, Gismondo M R, Murphy B R, Rappuoli R, Lanzavecchia A. (2004): An efficient method to make human monoclonal antibodies from memory B cells: potent neutralization of SARS coronavirus. Nat Med. 10(8):871-5.

Such immortalized B cell lines are particularly useful for the production of human antibodies. Accordingly, the present invention also provides a method for generating an antibody or a fragment thereof comprising a (heterologous) (polypeptide of interest, the method comprising the following steps:
(1) providing an engineered B lymphocyte or a B cell line according to the present invention as described herein, wherein the B lymphocyte comprises an edited immunoglobulin gene locus comprising a heterologous insert comprising a nucleotide sequence encoding a (poly)peptide of interest inserted in its switch region;
(2) culturing the engineered B lymphocyte or the B cell line; and
(3) isolating the antibody or the fragment thereof comprising the (heterologous) (poly)peptide of interest from the B cell culture.

As antibodies are secreted by B cells, isolation of antibodies is easy to achieve. Preferably, the isolated antibodies are purified. This means that the antibody will typically be present in a composition that is substantially free of other polypeptides e.g., where less than 90% (by weight), usually less than 60% and more usually less than 50% of the composition is made up of other polypeptides.

In addition, the method for generating an antibody or a fragment thereof according to the present invention preferably further comprises characterization of the antibody or antibody fragment, wherein characterization comprises Performing functional assays to determine the function of the antibody or antibody fragment;
Performing binding assays to determine the binding specificity of the antibody or antibody fragment and/or the binding partner/epitope recognized by the antibody or antibody fragment; and/or
Performing neutralization assays to determine the ability of the antibody or antibody fragment to neutralize a toxin or a pathogen.

Functional assays, binding assays and neutralization assays are known in the art. The skilled person will select the appropriate assay depending on the antibody's functionality. For example, if the antibody comprises a binding site (e.g., if the inserted (poly)peptide of interest comprises a binding site), the skilled person may perform a binding assay with the binding partner of said binding site.

In a further aspect, the present invention also provides an antibody obtainable by the method according for generating an antibody according to the present invention as described herein. In other words, the present invention also provides an antibody made by the method according for generating an antibody according to the present invention as described herein. Such an antibody comprises the (poly)peptide of interest as described above. Thereby, the (poly)peptide of interest may be located in the elbow region of the antibody as described above. Alternatively, the (poly)peptide of interest may also replace the variable region or the constant regions (e.g., of the heavy chain) of the antibody as described herein.

In a further aspect, the present invention also provides a composition comprising the engineered B lymphocyte according to the present invention or the antibody according to the present invention. Preferably, the composition further comprises a pharmaceutically acceptable pharmaceutically acceptable carrier, diluent and/or excipient. Accordingly, the composition is preferably a pharmaceutical composition.

Although the carrier or excipient may facilitate administration, it should not itself induce the production of antibodies harmful to the individual receiving the composition. Nor should it be toxic. Suitable carriers may be large, slowly metabolized macromolecules such as proteins, polypeptides, liposomes, polysaccharides, polylactic acids, polyglycolic acids, polymeric amino acids, amino acid copolymers and inactive virus particles. In general, pharmaceutically acceptable carriers in a pharmaceutical composition according to the present invention may be active components or inactive components.

Pharmaceutically acceptable salts can be used, for example mineral acid salts, such as hydrochlorides, hydrobromides, phosphates and sulphates, or salts of organic acids, such as acetates, propionates, malonates and benzoates.

Pharmaceutically acceptable carriers in a pharmaceutical composition may additionally contain liquids such as water, saline, glycerol and ethanol. Additionally, auxiliary substances, such as wetting or emulsifying agents or pH buffering substances, may be present in such compositions. Such carriers enable the pharmaceutical compositions to be formulated as tablets, pills, dragees, capsules, liquids, gels, syrups, slurries and suspensions, for ingestion by the subject.

Pharmaceutical compositions of the invention may be prepared in various forms. For example, the compositions may be prepared as injectables, either as liquid solutions or suspensions. Solid forms suitable for solution in, or suspension in, liquid vehicles prior to injection can also be prepared (e.g., a lyophilized composition, similar to Synagis™ and Herceptin™, for reconstitution with sterile water containing a preservative). The composition may be prepared e.g., as an ointment, cream or powder. The composition may be prepared for e.g., as a tablet or capsule, as a spray, or as a syrup (optionally flavored). The composition may be prepared e.g., as an inhaler, using a fine powder or a spray. The composition may be prepared e.g., as drops. The composition may be in kit form, designed such that a combined composition can be reconstituted, e.g. just prior to administration. For example, a lyophilized antibody may be provided in kit form with sterile water or a sterile buffer.

It is preferred that the active ingredient in the composition is an engineered B cell or an antibody according to the present invention. The composition may contain agents which protect the antibody from degradation or ensure viability of the B cell.

A thorough discussion of pharmaceutically acceptable carriers is available in Gennaro (2000) Remington: The Science and Practice of Pharmacy, 20th edition, ISBN: 0683306472.

Pharmaceutical compositions of the invention generally have a pH between 5.5 and 8.5, in some embodiments this may be between 6 and 8, and in other embodiments about 7. The pH may be maintained by the use of a buffer. The composition may be sterile and/or pyrogen free. The composition may be isotonic with respect to humans. In one embodiment pharmaceutical compositions of the invention are supplied in hermetically-sealed containers.

The composition may take the form of a suspension, solution or emulsion in an oily or aqueous vehicle and, in particular, it may contain formulatory agents, such as suspending, preservative, stabilizing and/or dispersing agents. Alternatively, the antibody molecule may be in dry form, for reconstitution before use with an appropriate sterile liquid.

The composition may comprise a vehicle, such as water or saline. A vehicle is typically understood to be a material that is suitable for storing, transporting, and/or administering a compound, such as a pharmaceutically active compound, in particular the antibodies according to the present invention. For example, the vehicle may be a physiologically acceptable liquid, which is suitable for storing, transporting, and/or administering a pharmaceutically active compound, in particular the antibodies according to the present invention.

The composition may be an aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives may be included, as required. The composition according to the present invention may be provided for example in a pre-filled syringe.

The composition as defined above may also be in a dosage form including, but not limited to, capsules, tablets, aqueous suspensions or solutions. In the case of tablets, carriers commonly used include lactose and corn starch. Lubricating agents, such as magnesium stearate, are also typically added. For capsule form, useful diluents include lactose and dried cornstarch. When aqueous suspensions are required, the active ingredient may be combined with emulsifying and suspending agents. If desired, certain sweetening, flavoring or coloring agents may also be added.

Further examples of carriers comprised by the composition include, but are not limited to, mineral oil, liquid petrolatum, white petrolatum, propylene glycol, polyoxyethylene, polyoxypropylene compound, emulsifying wax and water. Alternatively, the composition can be formulated in a suitable lotion or cream. In the context of the present invention, suitable carriers include, but are not limited to, mineral oil, sorbitan monostearate, polysorbate 60, cetyl esters wax, cetearyl alcohol, 2-octyldodecanol, benzyl alcohol and water.

In one embodiment, a composition of the invention may include antibodies of the invention, wherein the antibodies may make up at least 50% by weight (e.g., 60%, 70%, 75%, 80%, 85%, 90%, 95%, 96%, 97%, 98%, 99% or more) of the total protein in the composition. In such a composition, the antibodies are preferably in purified form.

Pharmaceutical compositions may include an antimicrobial particularly if packaged in a multiple dose format. They may comprise detergent e.g., a Tween (polysorbate), such as Tween 80. Detergents are generally present at low levels e.g., less than 0.01%. Compositions may also include sodium salts (e.g., sodium chloride) to give tonicity. For example, a concentration of 10±2 mg/ml NaCl is typical.

Further, pharmaceutical compositions may comprise a sugar alcohol (e.g., mannitol) or a disaccharide (e.g., sucrose or trehalose) e.g., at around 15-30 mg/ml (e.g., 25 mg/ml), particularly if they are to be lyophilized or if they include material which has been reconstituted from lyophilized material. The pH of a composition for lyophilization may be adjusted to between 5 and 8, or between 5.5 and 7, or around 6.1 prior to lyophilization.

The compositions of the invention may also comprise one or more immunoregulatory agents. In one embodiment, one or more of the immunoregulatory agents include(s) an adjuvant.

The composition comprising the engineered B cell or the antibody according to the present invention is preferably for use in medicine, i.e. as medication. In this context, the detailed description as described above for the medical use of an engineered B cell applies accordingly, for example regarding diseases to be treated.

Accordingly, the present invention also provides a method for immunotherapy comprising administration of the antibody according to the present invention, the engineered B cell according to the present invention or the composition according to the present invention to a subject in need thereof.

### BRIEF DESCRIPTION OF THE FIGURES

In the following a brief description of the appended figures will be given. The figures are intended to illustrate the present invention in more detail. However, they are not intended to limit the subject matter of the invention in any way.

FIG. 1 shows a schematic overview of AID mediated B cell engineering of the antibody switch region on chromosome 14 by integration of an extra exon element ((poly)peptide of interest) generating antibodies comprising a desired specificity ((poly)peptide of interest).

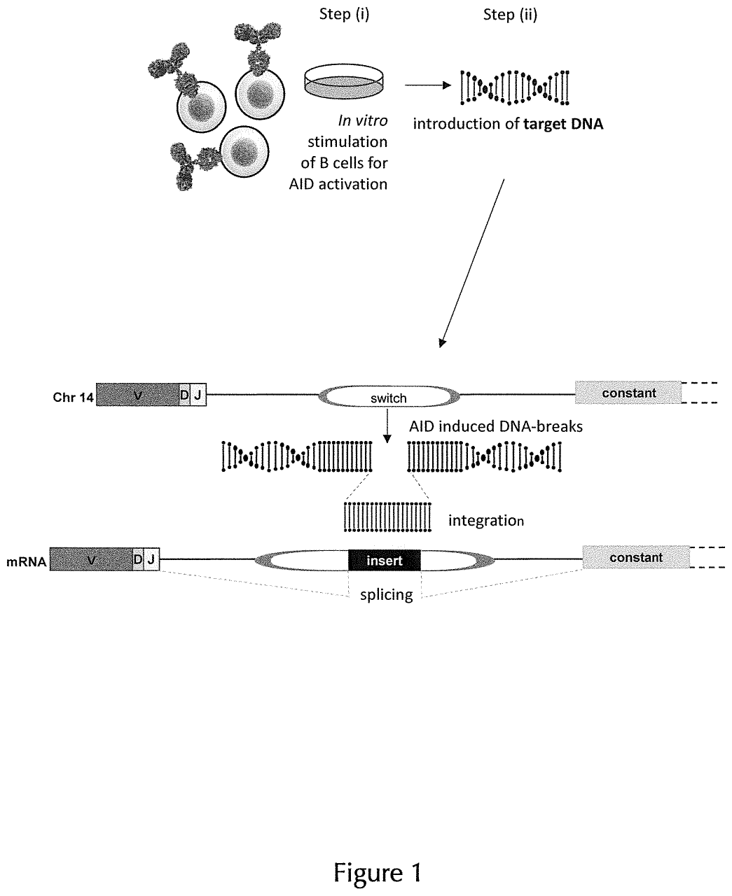

FIG. 2 shows schematic examples of engineered genes encoding antibody chains obtainable from B cells engineered according to the present invention and schematic drawings of the respective antibodies. (A) Examples including additional inserts in the antibody's elbow region (between the variable and constant regions). (B) Examples including a T2A protease cleavage site, e.g. to replace the original variable region of the antibody. V, D, J—original V, D, genes. Constant—original constant domain(s). T2A—introduced T2A cleavage site. V_{H}—introduced heavy chain variable region. V_{L}—introduced light chain variable region. Receptor domain—introduced receptor domain.

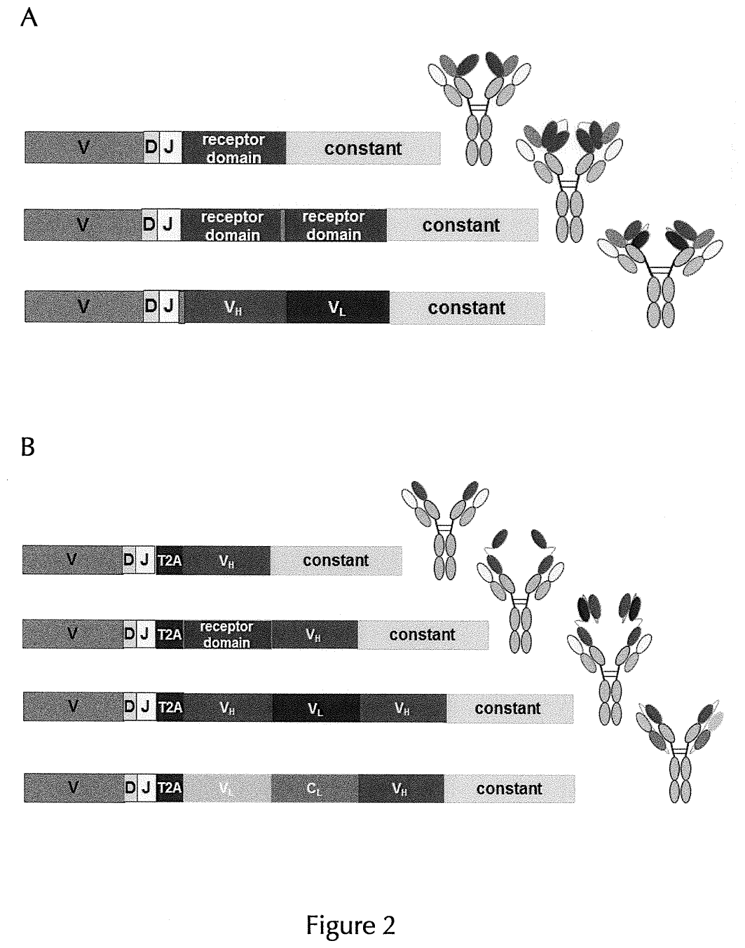

FIG. 3 provides a schematic overview over Examples (exp) for detecting genomic LAIR1 insertions (Example 1; exp 1), LAIR1-Ab expressing primary B cell production and selection by sorting (Example 2; exp 2) or by high throughput screening (Example 3; exp 3). The number of days indicates how many days after stimulation nucleofection was performed, the remarks in the column “nucleofection” indicate features of the nucleic acid used for nucleofection and the remarks in the column “screening” indicate what type of screening was performed.

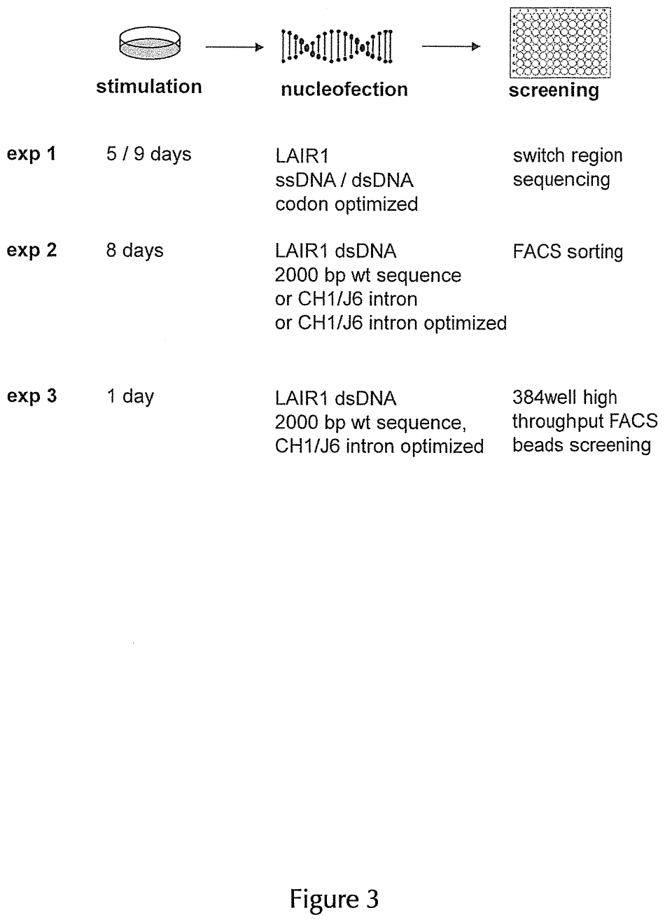

FIG. 4 shows for Example 1 (A) the design of switch region PCR and (B) the results of detection of codon optimized LAIR1 (including partial integrations) in long switch-μ-region PCR amplicons by MinION sequencing technology after nucleofection of double stranded (dsDNA) LAIR1 substrates.

FIG. 5 shows for Example 2 (A) LAIR1 and IgM surface co-staining of a B cell line generated according to the present invention, which expresses LAIR1-containing antibodies, selected post nucleofection by FACS sorting in comparison to negative (MME17) and positive (MMJS) control B cell lines. (B) Bead pull down and FACS analysis of artificial LAIR1-containing antibodies secreted by B cell lines nucleofected with a LAIR1 wildtype and a LAIR1 CH1/J6 intron optimized substrate.

FIG. 6 shows for Example 2 (A) LAIR1 and IgM specific western blots of culture supernatants. (B) PCR amplification using switch-μ-forward and LAIR1-reverse primers of genomic DNA isolated from engineered B cell lines expressing recombinant LAIR1-containing antibodies. (C) PCR product sequence alignment of switch-region and 5′ LAIR1-insert covering region highlighting switch region in gray, LAIR1 intron light gray with splice acceptor site in bold and LAIR1 exon in black.

FIG. 7 shows for Example 3 (A) frequency of engineered B cell lines expressing recombinant LAIR1-containing antibodies detected by high throughput screening of 60 000 and 35 000 cells, respectively. Cells were nucleofected with either LAIR1 wildtype substrate or a CH1/J6 intron optimized version. Screening conditions in II) were optimized by decreasing cell seeding numbers while increasing cultivation time to achieve higher antibody concentrations in culture supernatants. (B) Example of bead screenings of two 384 well culture plates measuring MFI ration of IgM captured by anti-LAIR1 versus control beads. Open circles show positive controls and the rectangle a culture secreting artificial LAIR1-containing antibodies.

FIG. 8 shows for Example 4 (A) H2AX staining indicating DNA double strand breaks after irradiation of PBMCs (FACS plots) and (B) primary B cells post CD40L/IL4 stimulation and AID induction. MFI=mean fluorescence intensity.

FIG. 9 shows for Example 4 (A) % of surviving and GFP expressing primary B cells two days after NEON nucleofection of a pMAX-GFP control plasmid. (B) FACS plots show gating strategies of mock nucleofectants and condition d) (2150V, 10 ms, 2 pules) which was used for further screening experiments.

FIG. 10 shows the principle and results for Example 5. (A) In vitro switch insertions are dependent on c-NHEJ. (B) naïve sorted B cells were stimulated with CD40L and IL4 and cultivated for 9 days in the presence of inhibitors for c-NHEJ (SCR7), a-NHEJ (Olaparib) or reverse transcriptases (ddl/AZT). Natural switch inserts were detected in 50,000 in vitro IgG+ switched B cells by MinION sequencing technology.

FIG. 11 shows a schematic representation of intron optimization for splice site recognition.

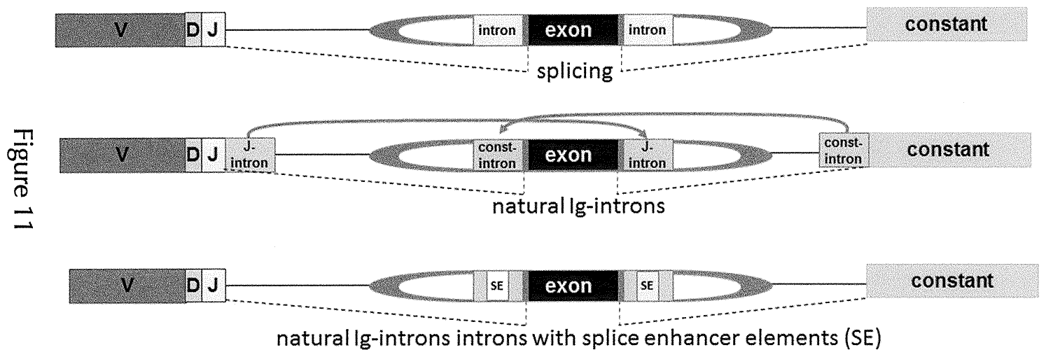

### EXAMPLES

In the following, particular examples illustrating various embodiments and aspects of the invention are presented. However, the present invention shall not to be limited in scope by the specific embodiments described herein. The following preparations and examples are given to enable those skilled in the art to more clearly understand and to practice the present invention. The present invention, however, is not limited in scope by the exemplified embodiments, which are intended as illustrations of single aspects of the invention only, and methods which are functionally equivalent are within the scope of the invention. Indeed, various modifications of the invention in addition to those described herein will become readily apparent to those skilled in the art from the foregoing description, accompanying figures and the examples below. All such modifications fall within the scope of the appended claims.

### Example 1: Generation of B Cells Engineered According to the Present Invention and Expressing Recombinant Antibodies

The underlying rationale of Examples 1-3 was to demonstrate that the immunoglobulin switch region of isolated human B cells can be targeted for genetic modification and subsequently results in production of recombinant antibodies. As an example, several experiments were successfully conducted to generate engineered human primary B cells producing an antibody with an inserted LAIR1 domain (Examples 1-3).

Methods:

B cell isolation, simulation and nucleofection. Primary human B cells were isolated from peripheral blood mononuclear cells (PBMCs) by magnetic cell sorting with anti-CD19 microbeads from Miltenyi Biotec. The 100 000/ml B cells were plated in 12 well. CD40L expressing, irradiated K562L cells were added to the B cells in a 1:2 ratio. Human recombinant IL4 was added at 16 ng/ml. The following day cells were re-stimulated with 8 ng/ml IL4. The nucleofection was either performed at day 1 after cell seeding, 4 h after IL4 re-stimulation or they were further cultivated, re-stimulated every 3 days with 8 ng/ml IL4 and nucleofected at indicated time points. For nucleofection the B cells were harvested and 2×10⁶ B cells were nucleofected with 1 μg DNA using a NEON® device according to manufacturers' instructions and 2150V, 10 ms and 2 pulses.

DNA nucleofection products. Codon optimized LAIR1 was ordered by gene synthesis from GenScript® using the company own codon optimization tool. For ssDNA generation, the “Long single strand DNA (LsODN) Preparation Kit” (funakoshi) was used. Codon optimized LAIR1 was cloned into pLSODN-1 vector. Restriction enzyme digestion of the vector was performed to generate either ssDNA or blunt/sticky-end dsDNA of codon optimized LAIR1.

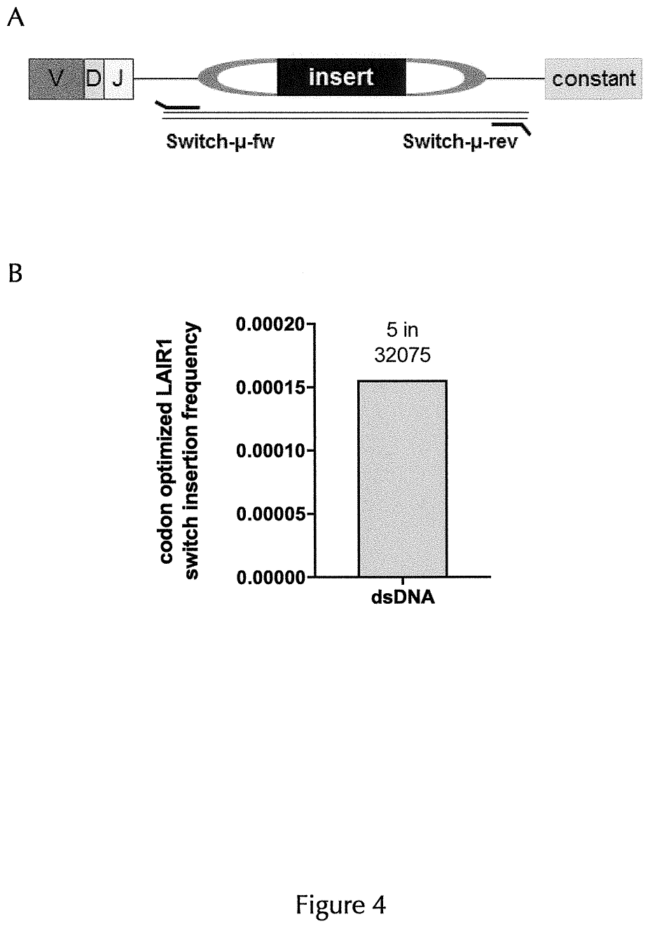

Sequence analysis. gDNA was isolated from nucleofected B cells 7 days after nucleofection using a commercial kit (QIAGEN). Switch region PCRs on gDNA were performed using LongAmp Taq Polymerase (New England Biolabs) in 50 μl reaction volumes with incubation for 3 min at 95° C., followed by 30 cycles of 95° C. for 40 s, 60° C. for 30 s, 65° C. for 3 min and a final extension for 10 min at 65° C. The upstream switch-μ forward primer S-μ-FW (cacccttgaaagtagcccatgccttcc; SEQ ID NO: 96) was combined with S-γ-REV (cctgcctcccagtgtcctgcattacttctg; SEQ ID NO: 97). Instead, the switch-μ region of nucleofected B cell gDNA was amplified combining the S-μ-FW primer with S-μ-REV (ggaacgcagtgtagactcagctgagg; SEQ ID NO: 98). The PCR reaction was performed using Herculase II Fusion DNA Polymerases (Agilent) with 1 M betaine and 3% DMSO in a 50 μl volume at 98° C. for 4 min followed by 30 cycles of 98° C. for 40 s, 58° C. for 30 s and 72° C. for 4 min, with a final extension for 10 min at 72° C. An overview of the design of switch region PCR is provided in FIG. 4 A. Size-selected, purified switch amplicons from oligoclonal B cell cultures were sequenced by MinION/Oxford Nanopore Technology (ONT). Barcodes were introduced by the addition of recommended BC-sequences to S-μ and S-γ primers and PCR amplification. The sequencing library was prepared using the Nanopore 2D sequencing kit SQK-LSK207, followed by loading onto Nanopore flow cells FLO-MIN106 and sequencing with the MinION Mk1B sequencer for up to 20 h.

The DNA substrates used in the first experiment comprised a ssDNA and dsDNA version with codon optimized LAIR1 exon and wildtype flanking intronic sequences having the following nucleotide sequence:

[SEQ ID NO: 99]
TTGTGAGCAAGTCTCAGGGTCCTCACTGTCAACTGGGAAAAAACTCTGC
AGTGATGAGAATCACATGCACGTAGAAGGTGCAGGAGGCGTGGGAATGT
TCTAAGGTTGGGCTGTGGTCATGGCTGCATAACTCTATAAAATTGCTAA
AATCCCTGAATTGTGATGCTAAAATGACGTGTGTGGCATGGTGACTTCC
TACAGTGGACGCTGAGATCCTGCTCTGCTTCCCTCCT [symbol] AAGATCTGCC
CAGACCCTCCATCTCGGCTGAGCCAGGCACCGTGATCCCCCTGGGGAGC
CATGTGACTTTCGTGTGCCGGGGCCCGGTTGGGGTTCAAACATTCCGCC
TGGAGAGGGACAGTAGATCCACATACAATGATACTGAAGATGTGTCTCA
AGCTAGTCCATCTGAGTCAGAGGCCAGATTCCGCATTGACTCAGTAAGA
GAAGGAAATGCCGGGCTTTATCGCTGCATCTATTATAAGCCCCCTAAAT
GGTCTGAGCAGAGTGACTACCTGGAGCTGCTGGTGAAAG [symbol] GAGGACGT
CACCTGGGCCCTGCCCCAGTCTCAGCTCGACCCTCGAGCTTGTCCCCAG
GT

(nucleotide sequence encoding a polypeptide of interest is shown underlined; 5′ and 3′ splice recognition sites are shown in bold and italics)

Results

In general, nucleofection with both, ssDNA and dsDNA substrates, resulted in successful integration of the nucleic acid substrate into the B cell genome. As an example, FIG. 4B shows results obtained with dsDNA.

### Example 2: Further Investigation of B Cell Lines Engineered According to the Present Invention and Expressing Recombinant Antibodies

To provide proof for productive insertion and expression of LAIR1 containing antibodies, primary B cells were nucleofected with a dsDNA LAIR1 wildtype substrate and, were screened by cell sorting for LAIR1 and IgM co-staining. As the natural LAIR1 receptor is downregulated after Epstein-Barr virus (EBV) immortalization, in this experimental setting EBV lines were generated to distinguish the natural receptor from an engineered B cell receptor.

To prepare LAIR1 wildtype (wt) products for nucleofection human wildtype LAIR1 was PCR amplified from human genomic DNA (gDNA) using the following primers (LAIR1_IN_FW ccacctccaaacggcaggcatcc (SEQ ID NO: 100); LAIR1_INTR_REV ccaaaggccgcatgaccatcacgc (SEQ ID NO: 101)). Chimeric DNA products containing a LAIR1 exon and introns deriving from the human immunoglobulin locus were generated by first amplifying single products with primers IgM-CH1-IN-fw cctcagctgagtctacactgcgttcc (SEQ ID NO: 102), IgM-CH1-IN-rev ctgaggacccgcaggacaaaagagaaaggg (SEQ ID NO: 103), J6-lN-fw ggtcaccgtctcctcaggtaagaatggcc (SEQ ID NO: 104), J6_IN-REV gccttttcagtttcggtcagcctcgc (SEQ ID NO: 105) and then fusing them by PCR with overlapping primers to a LAIR1 wt amplicon with LAIR1-CH1-FW gcgggtcctcagaagatctgcccagaccc (SEQ ID NO: 106) and LAIR1-J6-REV ggccattcttacctttcaccagcagctccagg (SEQ ID NO: 107). Optimized versions were generated using primers LAIR1-CH1-opt-FW gcgggtcctcaggggaagatctgcccagaccc (SEQ ID NO: 108), and LAIR1-J6-REV ggccattcttacctgaggagacggctttcaccagcagctccagg (SEQ ID NO: 109). To minimize mutations introduced by the polymerase during amplification, the Q5® High-Fidelity DNA Polymerase (New England Biolabs) with high proofreading activity was used applying the standard PCR amplification program.

B cells were isolated, stimulated and nucleofected as described above. One day after nucleofection, the B cells were immortalized with Epstein-Barr virus (EBV) by 4 h virus incubuation rotating at 37° C. as previously described (Traggiai E, Becker S, Subbarao K, Kolesnikova L, Uematsu Y, Gismondo M R, Murphy B R, Rappuoli R, Lanzavecchia A. An efficient method to make human monoclonal antibodies from memory B cells: potent neutralization of SARS coronavirus. Nat Med. 2004 August; 10(8):871-5. Epub 2004 Jul. 11) B cells were washed and plated in bulk at 1×10⁶/ml into 24 well cultures in the presence of CpG-DNA (2.5 μg/ml). One week after immortalization and downregulation of the B cell own LAIR1 wildtype receptor, the B cells were selected for LAIR1 and IgM co-expression by first labeling them with monoclonal anti-LAIR1 PE-conjugated (clone DX26, BD Bioscience, 550811) and anti-IgM APC-conjugated antibodies (Jackson ImmunoResearch, 109-606-129) followed by FACS-sorting. The cells which co-expressed LAIR1 and IgM were plated in 96U wells, expanded for two weeks and then repeatedly selected by FACS-sorting. Genomic analysis of gDNA isolated from the cell line and switch-μ region PCR amplification was performed as described above, followed by Sanger sequencing.

To confirm secretion of LAIR1 containing antibodies by EBV immortalized B cells, the culture supernatants were analyzed by western blot analysis. Supernatants were diluted in water and incubated with 4× sample loading buffer (Life Technologies) and 10× reducing agent (Life Technologies) for 10 min at 70° C. The samples were loaded to a precast gel with a 4-12% acrylamide gradient (Invitrogen). Proteins were transferred to PVDF membranes by the iBlot2 apparatus (Life Technologies) followed by blocking for 1 h at room temperature with 3% BSA in TBS. The membrane was incubated with different combinations of primary and secondary antibodies diluted in TBS/1% BSA for 1 h at room temperature with 2 sequential TBS incubations to wash the membrane between incubations. IgM isotypes were stained with 10 μg/ml unlabelled goat anti-human IgM (Southern Biotech, 2020-01) and 8 ng/ml donkey anti-goat HRP (Jackson ImmunoResearch, 705-036-147). LAIR1-containing antibodies were detected with a polyclonal goat anti-human LAIR1antibody (R&D) at 2 μg/ml was combined with secondary donkey anti-goat HRP. Membranes were developed with ECL-substrate on a Las4000 imager (General Electric Company).

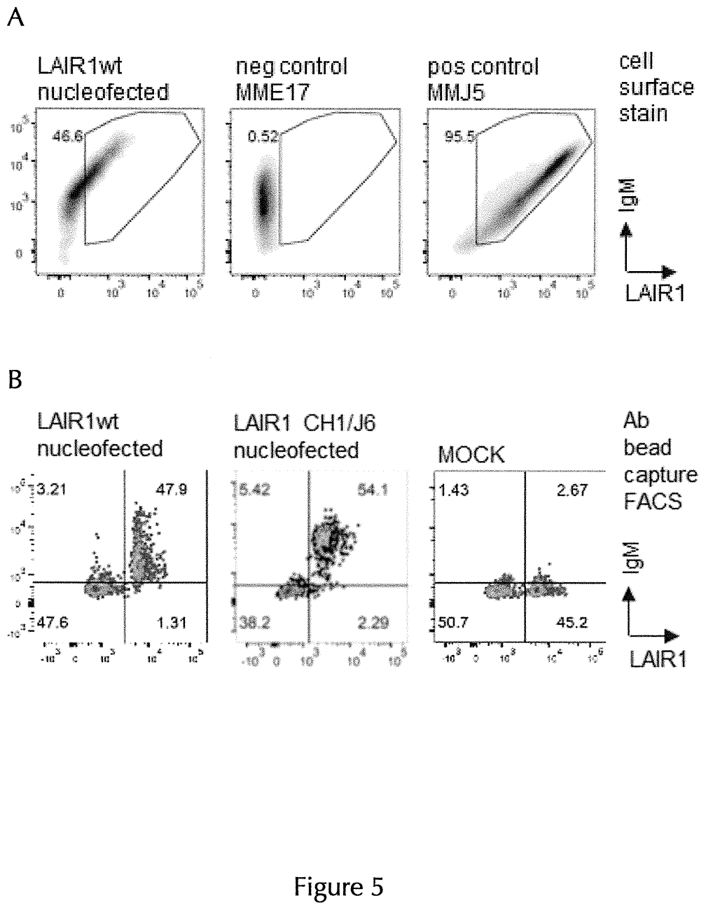

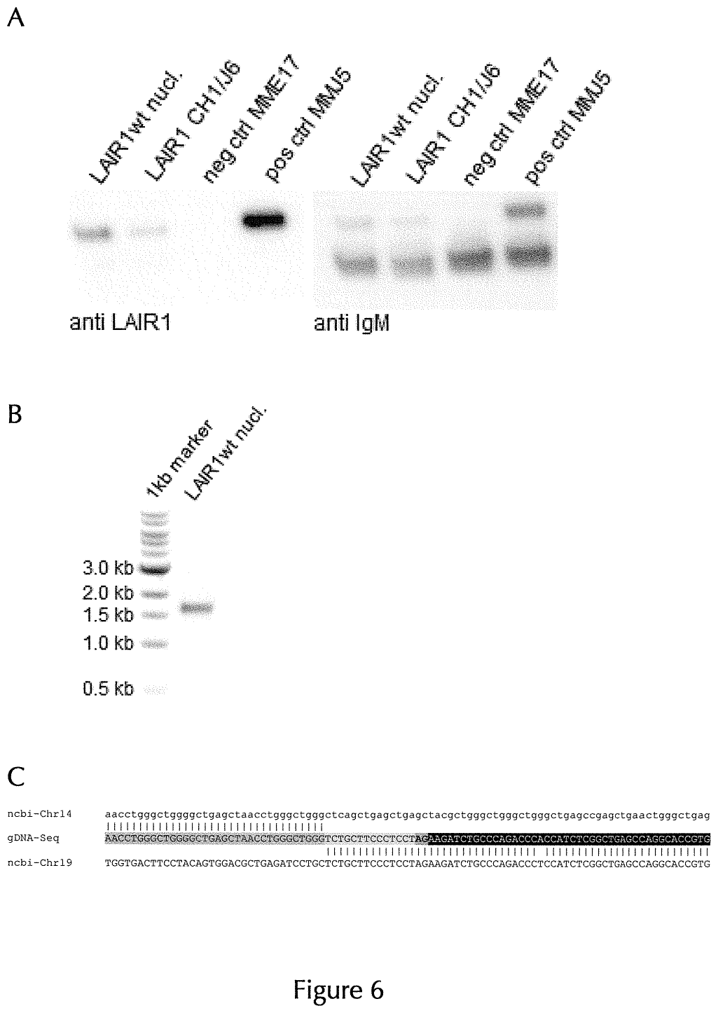

FIG. 5 shows the results of FACS analysis. FIG. 5A shows that the B cells were generated successfully expressing LAIR1-IgM on their surface. The integration of the LAIR1 domain into secreted antibodies was confirmed by a bead capture assay (FIG. 5B) and western blot analysis (FIG. 6A) as described above. Furthermore, successful integration was also achieved using a substrate where a LAIR1 wildtype exon was flanked by Immunoglobulin-locus intronic regions namely the J-segment downstream intron and the CH1-upstream intron (named LAIR1 CH1/J6) having the following sequence:

[SEO ID NO: 110]
CCTCAGCTGAGTCTACACTGCGTTCCCCATCACACTCACCCTCCCTATA
CTCACTCCCAGGCCTGGGTTGTCTGCCTGGGGAGACTTCAGGGTAGCTG
GAGTGTGACTGAGCTGGGGGCAGCAGAAGCTGGGCTGGAGGGACTCTAT
TGGCTGCCTGCGGGGTGTGTGGCTCCAGGCTTCACATTCAGGTATGCAA
CCTGGGCCCTCCAGCTGCATGTGCTGGGAGCTGAGTGTGTGCAGCACCT
ACGTGCTGATGCCTCGGGGGAAAGCAGGCCTGGTCCACCCAAACCTGAG
CCCTCAGCCATTCTGAGCAGGGAGCCAGGGGCAGTCAGGCCTCAGAGTG
CAGCAGGGCAGCCAGCTGAATGGTGGCAGGGATGGCTCAGCCTGCTCCA
GGAGACCCCAGGTCTGTCCAGGTGTTCAGTGCTGGGCCCTGCAGCAGGA
TGGGCTGAGGCCTGCAGCCCCAGCAGCCTTGGACAAAGACCTGAGGCCT
CACCACGGCCCCGCCACCCCTGATAGCCATGACAGTCTGGGCTTTGGAG
GCCTGCAGGTGGGCTCGGCCTTGGTGGGGCAGCCACAGCGGGACGCAAG
TAGTGAGGGCACTCAGAACGCCACTCAGCCCCGACAGGCAGGGCACGAG
GAGGCAGCTCCTCACCCTCCCTTTCTCTTTTGTCCTGCGGGTCCTC [symbol] A
AGATCTGCCCAGACCCTCCATCTCGGCTGAGCCAGGCACCGTGATCCCC
CTGGGGAGCCATGTGACTTTCGTGTGCCGGGGCCCGGTTGGGGTTCAAA
CATTCCGCCTGGAGAGGGACAGTAGATCCACATACAATGATACTGAAGA
TGTGTCTCAAGCTAGTCCATCTGAGTCAGAGGCCAGATTCCGCATTGAC
TCAGTAAGAGAAGGAAATGCCGGGCTTTATCGCTGCATCTATTATAAGC
CCCCTAAATGGTCTGAGCAGAGTGACTACCTGGAGCTGCTGGTGAAAG [symbol]
AAGAATGGCCACTCTAGGGCCTTTGTTTTCTGCTACTGCCTGTGGGG
TTTCCTGAGCATTGCAGGTTGGTCCTCGGGGCATGTTCCGAGGGGACCT
GGGCGGACTGGCCAGGAGGGGATGGGCACTGGGGTGCCTTGAGGATCTG
GGAGCCTCTGTGGATTTTCCGATGCCTTTGGAAAATGGGACTCAGGTTG
GGTGCGTCTGATGGAGTAACTGAGCCTGGGGGCTTGGGGAGCCACATTT
GGACGAGATGCCTGAACAAACCAGGGGTCTTAGTGATGGCTGAGGAATG
TGTCTCAGGAGCGGTGTCTGTAGGACTGCAAGATCGCTGCACAGCAGCG
AATCGTGAAATATTTTCTTTAGAATTATGAGGTGCGCTGTGTGTCAACC
TGCATCTTAAATTCTTTATTGGCTGGAAAGAGAACTGTCGGAGTGGGTG
AATCCAGCCAGGAGGGACGCGTAGCCCCGGTCTTGATGAGAGCAGGGTT
GGGGGCAGGGGTAGCCCAGAAACGGTGGCTGCCGTCCTGACAGGGGCTT
AGGGAGGCTCCAGGACCTCAGTGCCTTGAAGCTGGTTTCCATGAGAAAA
GGATTGTTTATCTTAGGAGGCATGCTTACTGTTAAAAGACAGGATATGT
TTGAAGTGGCTTCTGAGAAAAATGGTTAAGAAAATTATGACTTAAAAAT
GTGAGAGATTTTCAAGTATATTAATTTTTTTAACTGTCCAAGTATTTGA
AATTCTTATCATTTGATTAACACCCATGAGTGATATGTGTCTGGAATTG
AGGCCAAAGCAAGCTCAGCTAAGAAATACTAGCACAGTGCTGTCGGCCC
CGATGCGGGACTGCGTTTTGACCATCATAAATCAAGTTTATTTTTTTAA
TTAATTGAGCGAAGCTGGAAGCAGATGATGAATTAGAGTCAAGATGGCT
GCATGGGGGTCTCCGGCACCCACAGCAGGTGGCAGGAAGCAGGTCACCG
CGAGAG

(nucleotide sequence encoding a polypeptide of interest is shown underlined; 5′ and 3′ splice recognition sites are shown in bold and italics)

The genomic insertion of the LAIR1 wildtype sequence into the switch region was confirmed by a specific PCR reaction and sequence analysis (FIG. 6 B, C).

### Example 3: Further Investigation of B Cell Lines Engineered According to the Present Invention and Expressing Recombinant Antibodies

To evaluate the frequency of successfully nucleofected cells producing LAIR1-containing antibodies, 10-30 cells per well cultures in 384 well formats were screened by LAIR1-capture bead assay.

B cell isolation, stimulation, nucleofection and EBV immortalization was performed as described above. After virus incubation, the B cells were plated at 10 or 30 cells/well in presence of 25 000 irradiated, autologous PBMCs as feeder cells and CpG-DNA (2.5 μg/ml). After 2 weeks of cultivation the supernatants of the cells were analyzed for secretion of LAIR1-containing antibodies by a two-determinant bead-based immunoassay. Therefore, anti-goat IgG microbeads (Spherotech) were coated with either goat anti-human LAIR1 (R&D Systems, AF2664) or the control antibody goat anti-human EGF (R&D Systems, AF-259-NA) for 20 min at room temperature. SYBR Green I (ThermoFisher Scientific) was added at 40× to the LAIR1 antibody coating solution to distinguish LAIR1-coated from control beads. The beads were washed, mixed, and incubated with the supernatant of immortalized B cells for 30 min at room temperature. Bead captured, LAIR1 containing antibodies were detected using 2.5 μg/ml Alexa Fluor 647-conjugated donkey anti-human IgM (Jackson ImmunoResearch, 709-606-073).

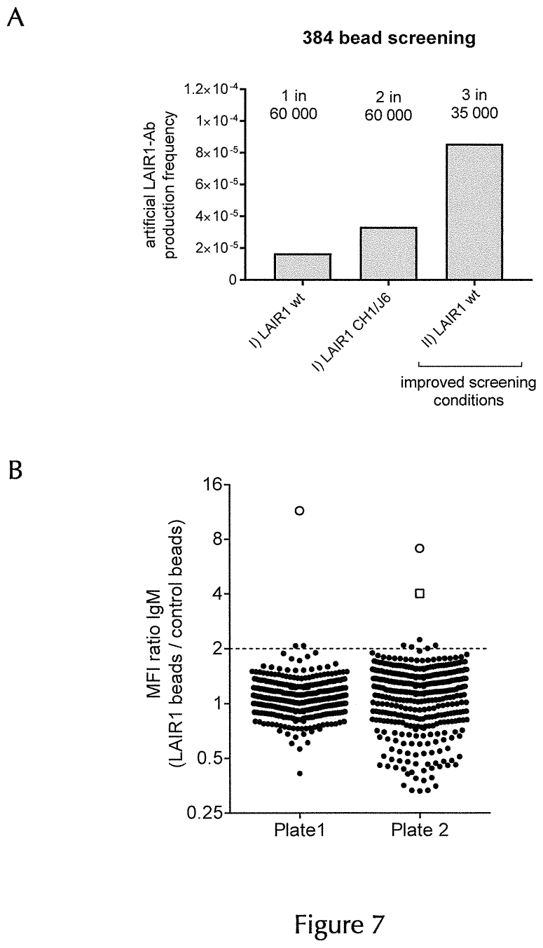

Results are shown in FIG. 7. The results confirm a frequency of one productive insertion in about 12 000 primary B cells (FIG. 7 A, B).

### Example 4: Optimization of Nucleofection Time Point and Condition

In this example the optimal time point and conditions of nucleofection was investigated.

B cell were isolated from PBMCs by Magnetic cell sorting with anti-CD19 beads and stimulated with CD40L expressing K562L cells and IL4 as described above. To assess the induction of DNA double strand breaks the cells were harvested at indicated time points, fixed with 3.7% formaldehyde, permabilized with 90% methanol and stored at −20° C. At the day of analysis, the cells were stained with rabbit anti-H2AX (Histone H3, clone D1H2, #12167S, cell signaling) at 0.25 μg/ml and analyzed by flow cytometry. To control for antibody staining specificity a staining with irradiated or untreated PBMCS was performed.

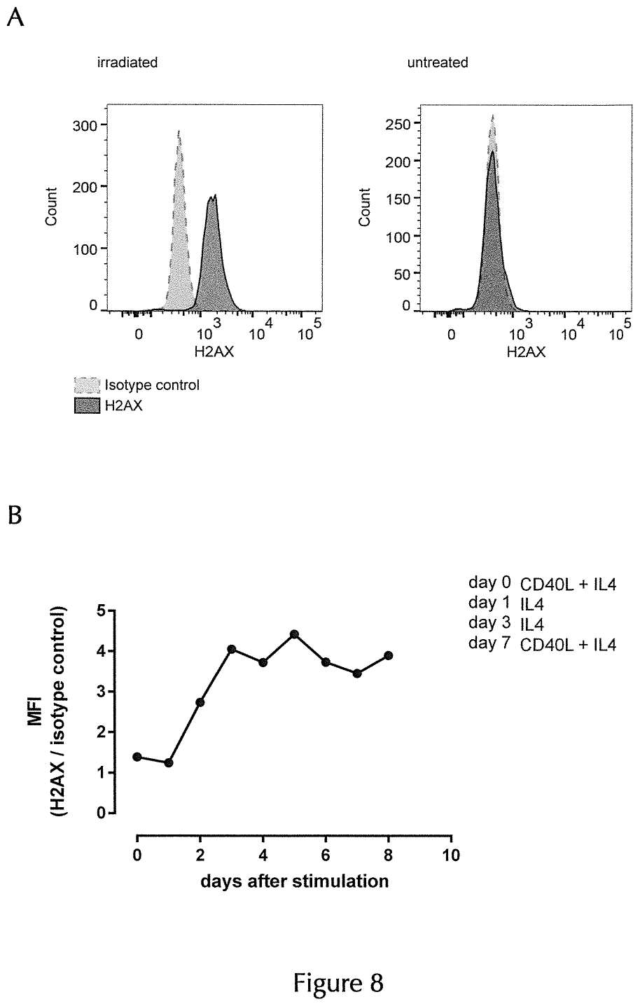

B cells were nucleofected 1-10 days after culture initiation. Results are shown in FIG. 8. As shown by staining of the H2AX histone marker in FIG. 8B, the maximum of DNA double strand breaks is achieved starting at days 2-3.

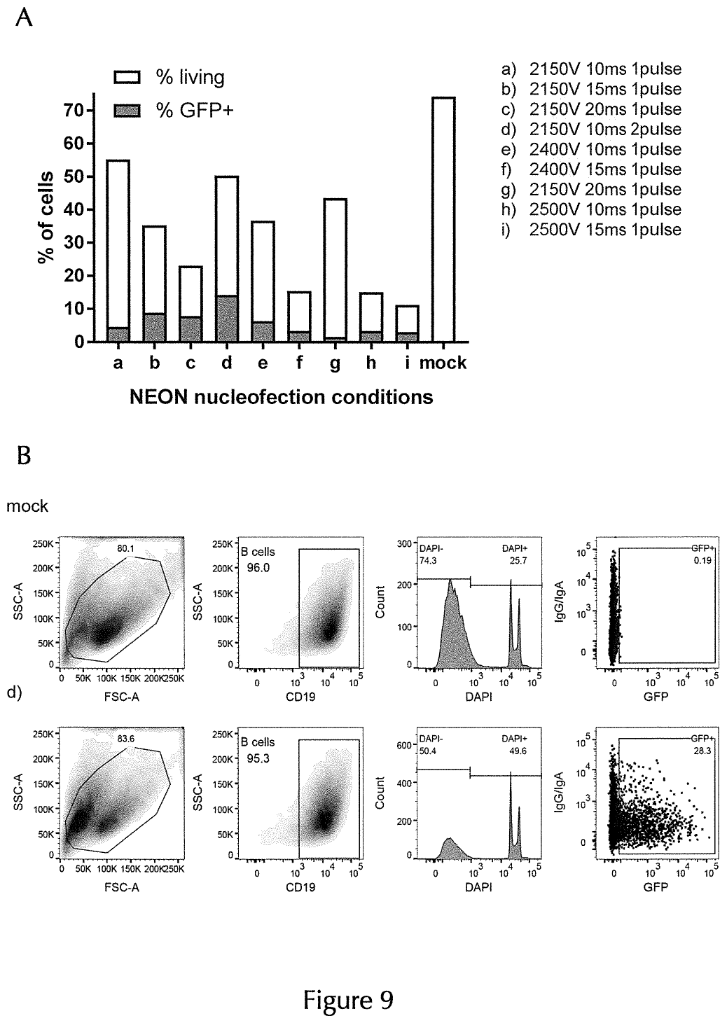

Next, B cells were nucleofected under distinct conditions with the Neon® Transfection System (Thermo Fisher Scientific) at 2150V 10 ms 1 pulse, 2150V 15 ms 1 pulse, 2150V 20 ms 1 pulse, 2150V 10 ms 2 pulse, 2400V 10 ms 1 pulse, 2400V 15 ms 1 pulse, 2150V 20 ms 1 pulse, 2500V 10 ms 1 pulse and 2500V 15 ms 1 pulse. Results are shown in FIG. 9. Under all conditions successful nucleofection was achieved. The best results were obtained using 2150V, 10 ms, 2 pulses.

### Example 5: Influence of c-NHEJ and a-EJ on Insert Acquisition in the Switch Region

To increase engineering efficiency, an in vitro system was used to study the influence of c-NHEJ and a-EJ on acquisition of natural inserts.

To this end, B cells were isolated by magnetic beads using anti-CD19 beads, followed by FACS-sorting and selection of naïve B cells (IgM⁺ IgD⁺ CD27⁻ IgG⁻ IgA⁻). Cells were plated in 48 well plates at concentrations of 50 000 B cells/ml and stimulated with 25 000 irradiated K562L/ml and 8 ng/ml IL4. The DNA repair inhibitor Olaparip (4 nM), SCR7 (100 nM) or DMSO (1:100) as control were added to the culture medium. At day 3 and day 6 the culture medium was replaced which fresh medium supplemented with IL4 and inhibitors. Cells were harvested at day 10 and stained with fluorescently labeled anti-CD19 and anti-IgG antibodies. Switched IgG B cell were sorted by flow cytometry and gDNA was isolation using a commercial kit. Genomic DNA of 50 000 sorted cells were used for γ-switch region PCR amplification and MINION sequencing as described above. Inserts frequencies were analyzed with a bioinformatics pipeline (Pieper K, Tan J, Piccoli L, Foglierini M, Barbieri S, Chen Y, Silacci-Fregni C, Wolf T, Jarrossay D, Anderle M, Abdi A, Ndungu F M, Doumbo O K, Traore B, Tran T M, Jongo S, Zenklusen I, Crompton P D, Daubenberger C, Bull P C, Sallusto F, Lanzavecchia A: Public antibodies to malaria antigens generated by two LAIR1 insertion modalities. Nature. 2017. Aug. 31; 548(7669):597-601).

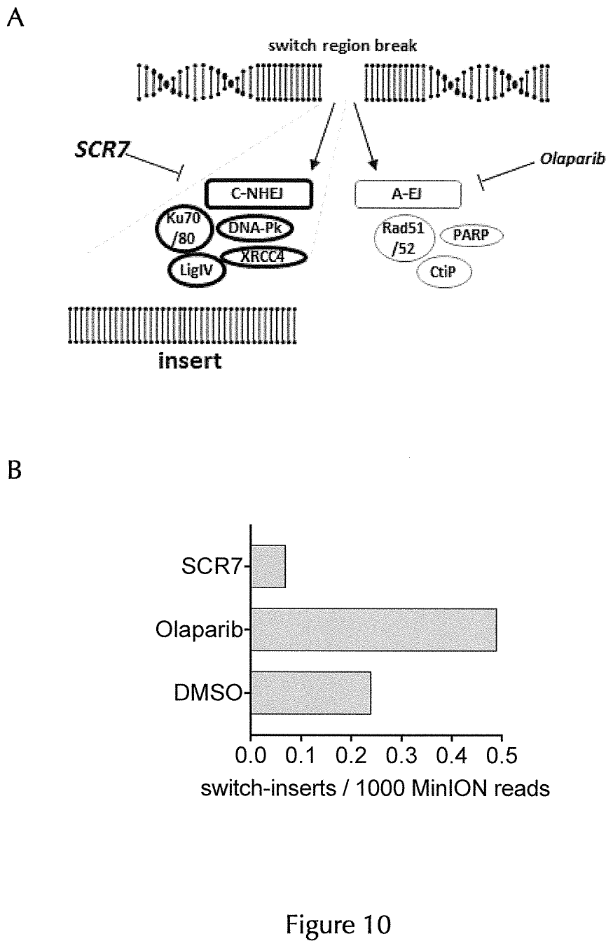

The general principle is shown in FIG. 10A and results are shown in FIG. 10B. The results show that natural switch inserts depend on the c-NHEJ DNA repair pathway as insert frequencies in presence of SCR7 are decreased while elevated when Olaparib was added to the culture medium (FIG. 10 A, B). Therefore, B cell engineering in presence of the inhibitor Olaparib can increase engineering efficiency. Likewise, the pre-incubation of the DNA substrate with the DNA-binding proteins Ku70/80, the first event in c-NHEJ mediated repair, and nucleofection of the DNA-Ku70/80 protein complex can increase the number of successful integrations (FIG. 10 A).

TABLE OF SEQUENCES AND SEQ ID NUMBERS (SEQUENCE LISTING):

| SEQ ID NO | Sequence | Remarks |
|---|---|---|
| SEQ ID NO: 1 | AGGTAAGT | 3′ splice site |
| SEQ ID NO: 2 | YNCTGAC | branch site |
| | wherein Y may be C or T and N may be any | |
| | nucleotide selected from A, G, C and T | |
| SEQ ID NO: 3 | GTAGTGAGGG | intronic splicing |
| SEQ ID NO: 4 | GTTGGTGGTT | intronic splicing |
| | | enhancer |
| SEQ ID NO: 5 | AGTTGTGGTT | intronic splicing |
| | | enhancer |
| SEQ ID NO: 6 | GTATTGGGTC | intronic splicing |
| | | enhancer |
| SEQ ID NO: 7 | AGTGTGAGGG | intronic splicing |
| | | enhancer |
| SEQ ID NO: 8 | GGGTAATGGG | intronic splicing |
| | | enhancer |
| SEQ ID NO: 9 | TCATTGGGGT | intronic splicing |
| | | enhancer |
| SEQ ID NO: 10 | GGTGGGGGTC | intronic splicing |
| | | enhancer |
| SEQ ID NO: 11 | GGTTTTGTTG | intronic splicing |
| | | enhancer |
| SEQ ID NO: 12 | TATACTCCCG | intronic splicing |
| | | enhancer |
| SEQ ID NO: 13 | GTATTCGATC | intronic splicing |
| | | enhancer |
| SEQ ID NO: 14 | GGGGGTAGG | intronic splicing |
| | | enhancer |
| SEQ ID NO: 15 | GTAGTTCCCT | intronic splicing |
| | | enhancer |
| SEQ ID NO: 16 | GTTAATAGTA | intronic splicing |
| | | enhancer |
| SEQ ID NO: 17 | TGCTGGTTAG | intronic splicing |
| | | enhancer |
| SEQ ID NO: 18 | ATAGGTAACG | intronic splicing |
| | | enhancer |
| SEQ ID NO: 19 | TCTGAATTGC | intronic splicing |
| | | enhancer |
| SEQ ID NO: 20 | TCTGGGTTTG | intronic splicing |
| | | enhancer |
| SEQ ID NO: 21 | CATTCTCTTT | intronic splicing |
| | | enhancer |
| SEQ ID NO: 22 | GTATTGGTGT | intronic splicing |
| | | enhancer |
| SEQ ID NO: 23 | GGAGGGTTT | intronic splicing |
| | | enhancer |
| SEQ ID NO: 24 | TTTAGATTTG | intronic splicing |
| | | enhancer |
| SEQ ID NO: 25 | ATAAGTACTG | intronic splicing |
| | | enhancer |
| SEQ ID NO: 26 | TAGTCTATTA | intronic splicing |
| | | enhancer |
| SEQ ID NO: 27 | CGAGGAGGCAGCTCCTCACCCTCCCTTTCTCTTT | intronic sequence |
| | TGTCCTGCGGGTCCTCAG | |
| SEQ ID NO: 28 | CGAAGGGGGCGGGAGTGGCGGGCACCGGGC | intronic sequence |
| | TGACACGTGTCCCTCACTGCAG | |
| SEQ ID NO: 29 | TCCGCCCACATCCACACCTGCCCCACCTCTGACT | intronic sequence |
| | CCCTTCTCTTGACTCCAG | |
| SEQ ID NO: 30 | CCACAGGCTGGTCCCCCCACTGCCCCGCCCTCA | intronic sequence |
| | CCACCATCTCTGTTCACAG | |
| SEQ ID NO: 31 | TGGGCCCAGCTCTGTCCCACACCGCGGTCACAT | intronic sequence |
| | GGCACCACCTCTCTTGCAG | |
| SEQ ID NO: 32 | GGACACCTTCTCTCCTCCCAGATTCCAGTAACTC | intronic sequence |
| | CCAATCTTCTCTCTGCAG | |
| SEQ ID NO: 33 | AGGGACAGGCCCCAGCCGGGTGCTGACACGTC | intronic sequence |
| | CACCTCCATCTCTTCCTCAG | |
| SEQ ID NO: 34 | GGCCCACCCTCTGCCCTGAGAGTGACCGCTGTA | intronic sequence |
| | CCAACCTCTGTCCCTACAG | |
| SEQ ID NO: 35 | TGGGCCCAGCTCTGTCCCACACCGCAGTCACAT | intronic sequence |
| | GGCGCCATCTCTCTTGCAG | |
| SEQ ID NO: 36 | AGATACCTTCTCTCTTCCCAGATCTGAGTAACTC | intronic sequence |
| | CCAATCTTCTCTCTGCAG | |
| SEQ ID NO: 37 | ACGCATCCACCTCCATCCCAGATCCCCGTAACTC | intronic sequence |
| | CCAATCTTCTCTCTGCAG | |
| SEQ ID NO: 38 | ACGCGTCCACCTCCATCCCAGATCCCCGTAACT | intronic sequence |
| | CCCAATCTTCTCTCTGCAG | |
| SEQ ID NO: 39 | ACGCATCCACCTCCATCCCAGATCCCCGTAACTC | intronic sequence |
| | CCAATCTTCTCTCTGCAG | |
| SEQ ID NO: 40 | ACGCATCCACCTCCATCCCAGATCCCCGTAACTC | intronic sequence |
| | CCAATCTTCTCTCTGCAG | |
| SEQ ID NO: 41 | GACCCACCCTCTGCCCTGGGAGTGACCGCTGT | intronic sequence |
| | GCCAACCTCTGTCCCTACAG | |
| SEQ ID NO: 42 | TGGGCCCAGCTCTGTCCCACACCGCGGTCACAT | intronic sequence |
| | GGCACCACCTCTCTTGCAG | |
| SEQ ID NO: 43 | AGACACCTTCTCTCCTCCCAGATCTGAGTAACTC | intronic sequence |
| | CCAATCTTCTCTCTGCAG | |
| SEQ ID NO: 44 | AGGGACAGGCCCCAGCCGGGTGCTGACGCATC | intronic sequence |
| | CACCTCCATCTCTTCCTCAG | |
| SEQ ID NO: 45 | GGCCCACCCTCTGCCCTGGGAGTGACCGCTGT | intronic sequence |
| | GCCAACCTCTGTCCCTACAG | |
| SEQ ID NO: 46 | GTGAGTCTGCTGTCTGGGGATAGCGGGGAGCC | intronic sequence |
| | AGGTGTACTGGGCCAGGCAA | |
| SEQ ID NO: 47 | GTGAGTCCCACTGCAGCCCCCTCCCAGTCTTCT | intronic sequence |
| | CTGTCCAGGCACCAGGCCA | |
| SEQ ID NO: 48 | GTAAGATGGCTTTCCTTCTGCCTCCTTTCTCTGG | intronic sequence |
| | GCCCAGCGTCCTCTGTCC | |
| SEQ ID NO: 49 | GTGAGTCCTCACAACCTCTCTCCTGCTTTAACTC | intronic sequence |
| | TGAAGGGTTTTGCTGCAT | |
| SEQ ID NO: 50 | GTGAGTCCTCACCACCCCCTCTCTGAGTCCACTT | intronic sequence |
| | AGGGAGACTCAGCTTGCC | |
| SEQ ID NO: 51 | GTAAGAATGGCCACTCTAGGGCCTTTGTTTTCTG | intronic sequence |
| | CTACTGCCTGTGGGGTTT | |
| SEQ ID NO: 52 | CATGGTGACTTCCTACAGTGGACGCTGAGATCC | intronic sequence |
| | TGCTCTGCTTCCCTCCTAG | |
| SEQ ID NO: 53 | GTGAGGACGTCACCTGGGCCCTGCCCCAGTCT | intronic sequence |
| | CAGCTCGACCCTCGAGCTTG | |
| SEQ ID NO: 54 | LEVLFQGP | cleavage tag |
| SEQ ID NO: 55 | DDDDK | cleavage tag |
| SEQ ID NO: 56 | IEGR | cleavage tag |
| SEQ ID NO: 57 | ENLYFQG | cleavage tag |
| SEQ ID NO: 58 | LVPRGS | cleavage tag |
| SEQ ID NO: 59 | DX₁EX₂NPGP | self-processing site |
| | wherein X₁ is Val or Ile, and | |
| | X₂ may be any (naturally occurring) amino acid | |
| SEQ ID NO: 60 | EGRGSLLTCGDVEENPGP | self-processing site |
| SEQ ID NO: 61 | VKQTLNFDLLKLAGDVESNPGP | self-processing site |
| SEQ ID NO: 62 | ATNFSLLKQAGDVEENPGP | self-processing site |
| SEQ ID NO: 63 | GSGATNFSLLKQAGDVEENPGP | self-processing site |
| SEQ ID NO: 64 | RKRRGSGATNFSLLKQAGDVEENPGP | self-processing site |
| SEQ ID NO: 65 | SAWSHPQFEKGGGSGGGSGGSAWSHPQFEK | twin StrepTag aa |
| SEQ ID NO: 66 | GLNDIFEAQKIEWHE | AviTag |
| SEQ ID NO: 67 | KRRWKKNFIAVSAANRFKKISSSGAL | Calmodulin-tag |
| SEQ ID NO: 68 | EEEEEE | polyglutamate tag |
| SEQ ID NO: 69 | GAPVPYPDPLEPR | E-tag |
| SEQ ID NO: 70 | DYKDDDDK | FLAG-tag |
| SEQ ID NO: 71 | YPYDVPDYA | HA-tag |
| SEQ ID NO: 72 | HHHHHH | His-tag |
| SEQ ID NO: 73 | EQKLISEEDL | Myc-tag |
| SEQ ID NO: 74 | TKENPRSNQEESYDDNES | NE-tag |
| SEQ ID NO: 75 | KETAAAKFERQHMDS | S-tag |
| SEQ ID NO: 76 | MDEKTTGWRGGHVVEGLAGELEQLRARLEHHPQ | SBP-tag |
| | GQREP | |
| SEQ ID NO: 77 | SLAELLNAGLGGS | Softag 1 |
| SEQ ID NO: 78 | TQDPSRVG | Softag 3 |
| SEQ ID NO: 79 | WSHPQFEK | Strep-tag |
| SEQ ID NO: 80 | CCPGCC | TC tag |
| SEQ ID NO: 81 | GKPIPNPLLGLDST | V5 tag |
| SEQ ID NO: 82 | YTDIEMNRLGK | VSV-tag |
| SEQ ID NO: 83 | DLYDDDDK | Xpress tag |
| SEQ ID NO: 84 | TDKDMTITFTNKKDAE | Isopeptag |
| SEQ ID NO: 85 | AHIVMVDAYKPTK | SpyTag |
| SEQ ID NO: 86 | KLGDIEFIKVNK | SnoopTag |
| SEQ ID NO: 87 | EVHTNQDPLD | Ty1 tag |
| SEQ ID NO: 88 | EDLPRPSISAEPGTVIPLGSHVTFVCRGPVGVQTFRL | mutated LAIR1 |
| | ERERNYLYSDTEDVSQTSPSESEARFRIDSVNAGNA | fragment aa |
| | GLFRCIYYKSRKWSEQSDYLELVVK | |
| SEQ ID NO: 89 | DSPDRPWNPPTFSPALLVVTEGDNATFTCSFSNTSE | PD-1 fragment aa |
| | SFVLNWYRMSPSNQTDKLAAFPEDRSQPGQDCRF | |
| | RVTQLPNGRDFHMSVVRARRNDSGTYLCGAISLAP | |
| | KAQIKESLRAELRVT | |
| SEQ ID NO: 90 | EQVSTPEIKVLNKTQENGTCTLILGCTVEKGDHVAY | SLAM fragment aa |
| | SWSEKAGTHPLNPANSSHLLSLTLGPQHADNIYICT | |
| | VSNPISNNSQTFSPWPGCRTDPS | |
| SEQ ID NO: 91 | MAQVQLVESGGGLVQAGGSLTLSCAASGSTSRSY | T3-VHH aa |
| | ALGWFRQAPGKEREFVAHVGQTAEFAQGRFTISR | |
| | DFAKNTVSLQMNDLKSDDTAIYYCVASNRGWSPS | |
| | RVSYWGQGTQVTVSS | |
| SEQ ID NO: 92 | QITLKESGPTLVKPTQTLTLTCTFSGFSLSTSRVGVG | TT39.7-scFv aa |
| | WIRQPPGKALEWLSLIYWDDEKHYSPSLKNRVTISK | |
| | DSSKNQVVLTLTDMDPVDTGTYYCAHRGVDTSG | |
| | WGFDYWGQGALVTVSSGGGGSGGGGSGGGGS | |
| | QSALTQPASVSGSPGQSITISCSGAGSDVGGHNFV | |
| | SWYQQYPGKAPKLMIYDVKNRPSGVSYRFSGSKSG | |
| | YTASLTISGLQAEDEATYFCSSYSSSSTLIIFGGGTRLT | |
| | VL | |
| SEQ ID NO: 93 | QVQLQESGGGLVQPGGSLRLSCAASGFTLDYYYIG | F4-VHH aa |
| | WFRQAPGKEREAVSCISGSSGSTYYPDSVKGRFTISR | |
| | DNAKNTVYLQMNSLKPEDTAVYYCATIRSSSWGG | |
| | CVHYGMDYWGKGTQVTVSS | |
| SEQ ID NO: 94 | EVQLVESGGGLVKPGGSLRLSCAASGFTFSSYSMN | MPE8-scFv aa |
| | WVRQAPGKGLEWVSSISASSSYSDYADSAKGRFTIS | |
| | RDNAKTSLFLQMNSLRAEDTAIYFCARARATGYSSI | |
| | TPYFDIWGQGTLVTVSSGGGGSGGGGSGGGGSQ | |
| | SVVTQPPSVSGAPGQRVTISCTGSSSNIGAGYDVH | |
| | WYQQLPGTAPKLLIYDNNNRPSGVPDRFSASKSGT | |
| | SASLAITGLQAEDEADYYCQSYDRNLSGVFGTGTK | |
| | VTVL | |
| SEQ ID NO: 95 | TGGTTGCTGACTAATTGAGATGCATGCTTTGCAT | SV40 nuclear |
| | ACTTCTGCCTGCTGGGGAGCCTGGGGACTTTCC | localization signal |
| | ACACCTGGTTGCTGACTAATTGAGATGCATGCTT | |
| | TGCATACTTCTGCCTGCTGGGGAGCCTGGGGA | |
| | CTTTCCACACC | |
| SEQ ID NO: 96 | cacccttgaaagtagcccatgccttcc | primer |
| SEQ ID NO: 97 | cctgcctcccagtgtcctgcattacttctg | primer |
| SEQ ID NO: 98 | ggaacgcagtgtagactcagctgagg | primer |
| SEQ ID NO: 99 | TTGTGAGCAAGTCTCAGGGTCCTCACTGTCAAC | DNA substrate |
| | TGGGAAAAAACTCTGCAGTGATGAGAATCACAT | |
| | GCACGTAGAAGGTGCAGGAGGCGTGGGAATG | |
| | TTCTAAGGTTGGGCTGTGGTCATGGCTGCATAA | |
| | CTCTATAAAATTGCTAAAATCCCTGAATTGTGAT | |
| | GCTAAAATGACGTGTGTGGCATGGTGACTTCCT | |
| | ACAGTGGACGCTGAGATCCTGCTCTGCTTCCCT | |
| | CCTAGAAGATCTGCCCAGACCCTCCATCTCGGC | |
| | TGAGCCAGGCACCGTGATCCCCCTGGGGAGCC | |
| | ATGTGACTTTCGTGTGCCGGGGCCCGGTTGGG | |
| | GTTCAAACATTCCGCCTGGAGAGGGACAGTAG | |
| | ATCCACATACAATGATACTGAAGATGTGTCTCAA | |
| | GCTAGTCCATCTGAGTCAGAGGCCAGATTCCGC | |
| | ATTGACTCAGTAAGAGAAGGAAATGCCGGGCTT | |
| | TATCGCTGCATCTATTATAAGCCCCCTAAATGGT | |
| | CTGAGCAGAGTGACTACCTGGAGCTGCTGGTG | |
| | AAAGGTGAGGACGTCACCTGGGCCCTGCCCCA | |
| | GTCTCAGCTCGACCCTCGAGCTTGTCCCCAGGT | |
| SEQ ID NO: 100 | ccacctccaaacggcaggcatcc | primer |
| SEQ ID NO: 101 | ccaaaggccgcatgaccatcacgc | primer |
| SEQ ID NO: 102 | cctcagctgagtctacactgcgttcc | primer |
| SEQ ID NO: 103 | ctgaggacccgcaggacaaaagagaaaggg | primer |
| SEQ ID NO: 104 | ggtcaccgtctcctcaggtaagaatggcc | primer |
| SEQ ID NO: 105 | gccttttcagtttcggtcagcctcgc | primer |
| SEQ ID NO: 106 | gcgggtcctcagaagatctgcccagaccc | primer |
| SEQ ID NO: 107 | ggccattcttacctttcaccagcagctccagg | primer |
| SEQ ID NO: 108 | gcgggtcctcaggggaagatctgcccagaccc | primer |
| SEQ ID NO: 109 | ggccattcttacctgaggagacggctttcaccagcagctccagg | primer |
| SEQ ID NO: 110 | CCTCAGCTGAGTCTACACTGCGTTCCCCATCACACTC | DNA substrate |
| | ACCCTCCCTATACTCACTCCCAGGCCTGGGTTGTCTG | |
| | CCTGGGGAGACTTCAGGGTAGCTGGAGTGTGACTG | |
| | AGCTGGGGGCAGCAGAAGCTGGGCTGGAGGGACT | |
| | CTATTGGCTGCCTGCGGGGTGTGTGGCTCCAGGCTT | |
| | CACATTCAGGTATGCAACCTGGGCCCTCCAGCTGCAT | |
| | GTGCTGGGAGCTGAGTGTGTGCAGCACCTACGTGCT | |
| | GATGCCTCGGGGGAAAGCAGGCCTGGTCCACCCAA | |
| | ACCTGAGCCCTCAGCCATTCTGAGCAGGGAGCCAGG | |
| | GGCAGTCAGGCCTCAGAGTGCAGCAGGGCAGCCAG | |
| | CTGAATGGTGGCAGGGATGGCTCAGCCTGCTCCAG | |
| | GAGACCCCAGGTCTGTCCAGGTGTTCAGTGCTGGGC | |
| | CCTGCAGCAGGATGGGCTGAGGCCTGCAGCCCCAG | |
| | CAGCCTTGGACAAAGACCTGAGGCCTCACCACGGCC | |
| | CCGCCACCCCTGATAGCCATGACAGTCTGGGCTTTG | |
| | GAGGCCTGCAGGTGGGCTCGGCCTTGGTGGGGCAG | |
| | CCACAGCGGGACGCAAGTAGTGAGGGCACTCAGAA | |
| | CGCCACTCAGCCCCGACAGGCAGGGCACGAGGAGG | |
| | CAGCTCCTCACCCTCCCTTTCTCTTTTGTCCTGCGGGT | |
| | CCTCAGAAGATCTGCCCAGACCCTCCATCTCGGCTGA | |
| | GCCAGGCACCGTGATCCCCCTGGGGAGCCATGTGA | |
| | CTTTCGTGTGCCGGGGCCCGGTTGGGGTTCAAACAT | |
| | TCCGCCTGGAGAGGGACAGTAGATCCACATACAATG | |
| | ATACTGAAGATGTGTCTCAAGCTAGTCCATCTGAGTC | |
| | AGAGGCCAGATTCCGCATTGACTCAGTAAGAGAAGG | |
| | AAATGCCGGGCTTTATCGCTGCATCTATTATAAGCCC | |
| | CCTAAATGGTCTGAGCAGAGTGACTACCTGGAGCTG | |
| | CTGGTGAAAGGTAAGAATGGCCACTCTAGGGCCTTT | |
| | GTTTTCTGCTACTGCCTGTGGGGTTTCCTGAGCATTG | |
| | CAGGTTGGTCCTCGGGGCATGTTCCGAGGGGACCT | |
| | GGGCGGACTGGCCAGGAGGGGATGGGCACTGGGG | |
| | TGCCTTGAGGATCTGGGAGCCTCTGTGGATTTTCCGA | |
| | TGCCTTTGGAAAATGGGACTCAGGTTGGGTGCGTCT | |
| | GATGGAGTAACTGAGCCTGGGGGCTTGGGGAGCCA | |
| | CATTTGGACGAGATGCCTGAACAAACCAGGGGTCTT | |
| | AGTGATGGCTGAGGAATGTGTCTCAGGAGCGGTGTC | |
| | TGTAGGACTGCAAGATCGCTGCACAGCAGCGAATCG | |
| | TGAAATATTTTCTTTAGAATTATGAGGTGCGCTGTGTG | |
| | TCAACCTGCATCTTAAATTCTTTATTGGCTGGAAAGAG | |
| | AACTGTCGGAGTGGGTGAATCCAGCCAGGAGGGAC | |
| | GCGTAGCCCCGGTCTTGATGAGAGCAGGGTTGGGG | |
| | GCAGGGGTAGCCCAGAAACGGTGGCTGCCGTCCTG | |
| | ACAGGGGCTTAGGGAGGCTCCAGGACCTCAGTGCC | |
| | TTGAAGCTGGTTTCCATGAGAAAAGGATTGTTTATCTT | |
| | AGGAGGCATGCTTACTGTTAAAAGACAGGATATGTTT | |
| | GAAGTGGCTTCTGAGAAAAATGGTTAAGAAAATTATG | |
| | ACTTAAAAATGTGAGAGATTTTCAAGTATATTAATTTTT | |
| | TTAACTGTCCAAGTATTTGAAATTCTTATCATTTGATTA | |
| | ACACCCATGAGTGATATGTGTCTGGAATTGAGGCCA | |
| | AAGCAAGCTCAGCTAAGAAATACTAGCACAGTGCTG | |
| | TCGGCCCCGATGCGGGACTGCGTTTTGACCATCATA | |
| | AATCAAGTTTATTTTTTTAATTAATTGAGCGAAGCTGG | |
| | AAGCAGATGATGAATTAGAGTCAAGATGGCTGCATG | |
| | GGGGTCTCCGGCACCCACAGCAGGTGGCAGGAAGC | |
| | AGGTCACCGCGAGAG | |
| SEQ ID NO: 111 | AAGATCTGCCCAGACCCTCCATCTCGGCTGAGC | Nucleotide sequence |
| | CAGGCACCGTGATCCCCCTGGGGAGCCATGTG | encoding (poly)peptide |
| | ACTTTCGTGTGCCGGGGCCCGGTTGGGGTTCA | of interest |
| | AACATTCCGCCTGGAGAGGGACAGTAGATCCA | |
| | CATACAATGATACTGAAGATGTGTCTCAAGCTA | |
| | GTCCATCTGAGTCAGAGGCCAGATTCCGCATTG | |
| | ACTCAGTAAGAGAAGGAAATGCCGGGCTTTATC | |
| | GCTGCATCTATTATAAGCCCCCTAAATGGTCTGA | |
| | GCAGAGTGACTACCTGGAGCTGCTGGTGAAAG | |
| SEQ ID NO: 112 | TTGTGAGCAAGTCTCAGGGTCCTCACTGTCAAC | intronic sequence |
| | TGGGAAAAAACTCTGCAGTGATGAGAATCACAT | |
| | GCACGTAGAAGGTGCAGGAGGCGTGGGAATG | |
| | TTCTAAGGTTGGGCTGTGGTCATGGCTGCATAA | |
| | CTCTATAAAATTGCTAAAATCCCTGAATTGTGAT | |
| | GCTAAAATGACGTGTGTGGCATGGTGACTTCCT | |
| | ACAGTGGACGCTGAGATCCTGCTCTGCTTCCCT | |
| | CCTAG | |
| SEQ ID NO: 113 | GTGAGGACGTCACCTGGGCCCTGCCCCAGTCT | intronic sequence |
| | CAGCTCGACCCTCGAGCTTGTCCCCAGGT | |
| SEQ ID NO: 114 | CCTCAGCTGAGTCTACACTGCGTTCCCCATCACA | intronic sequence |
| | CTCACCCTCCCTATACTCACTCCCAGGCCTGGGT | |
| | TGTCTGCCTGGGGAGACTTCAGGGTAGCTGGA | |
| | GTGTGACTGAGCTGGGGGCAGCAGAAGCTGG | |
| | GCTGGAGGGACTCTATTGGCTGCCTGCGGGGT | |
| | GTGTGGCTCCAGGCTTCACATTCAGGTATGCAA | |
| | CCTGGGCCCTCCAGCTGCATGTGCTGGGAGCT | |
| | GAGTGTGTGCAGCACCTACGTGCTGATGCCTCG | |
| | GGGGAAAGCAGGCCTGGTCCACCCAAACCTGA | |
| | GCCCTCAGCCATTCTGAGCAGGGAGCCAGGGG | |
| | CAGTCAGGCCTCAGAGTGCAGCAGGGCAGCCA | |
| | GCTGAATGGTGGCAGGGATGGCTCAGCCTGCT | |
| | CCAGGAGACCCCAGGTCTGTCCAGGTGTTCAGT | |
| | GCTGGGCCCTGCAGCAGGATGGGCTGAGGCCT | |
| | GCAGCCCCAGCAGCCTTGGACAAAGACCTGAG | |
| | GCCTCACCACGGCCCCGCCACCCCTGATAGCCA | |
| | TGACAGTCTGGGCTTTGGAGGCCTGCAGGTGG | |
| | GCTCGGCCTTGGTGGGGCAGCCACAGCGGGA | |
| | CGCAAGTAGTGAGGGCACTCAGAACGCCACTC | |
| | AGCCCCGACAGGCAGGGCACGAGGAGGCAGC | |
| | TCCTCACCCTCCCTTTCTCTTTTGTCCTGCGGGTC | |
| | CTCAG | |
| SEQ ID NO: 115 | GTAAGAATGGCCACTCTAGGGCCTTTGTTTTCTG | intronic sequence |
| | CTACTGCCTGTGGGGTTTCCTGAGCATTGCAGG | |
| | TTGGTCCTCGGGGCATGTTCCGAGGGGACCTG | |
| | GGCGGACTGGCCAGGAGGGGATGGGCACTGG | |
| | GGTGCCTTGAGGATCTGGGAGCCTCTGTGGATT | |
| | TTCCGATGCCTTTGGAAAATGGGACTCAGGTTG | |
| | GGTGCGTCTGATGGAGTAACTGAGCCTGGGGG | |
| | CTTGGGGAGCCACATTTGGACGAGATGCCTGA | |
| | ACAAACCAGGGGTCTTAGTGATGGCTGAGGAA | |
| | TGTGTCTCAGGAGCGGTGTCTGTAGGACTGCAA | |
| | GATCGCTGCACAGCAGCGAATCGTGAAATATTT | |
| | TCTTTAGAATTATGAGGTGCGCTGTGTGTCAACC | |
| | TGCATCTTAAATTCTTTATTGGCTGGAAAGAGAA | |
| | CTGTCGGAGTGGGTGAATCCAGCCAGGAGGGA | |
| | CGCGTAGCCCCGGTCTTGATGAGAGCAGGGTT | |
| | GGGGGCAGGGGTAGCCCAGAAACGGTGGCTG | |
| | CCGTCCTGACAGGGGCTTAGGGAGGCTCCAGG | |
| | ACCTCAGTGCCTTGAAGCTGGTTTCCATGAGAA | |
| | AAGGATTGTTTATCTTAGGAGGCATGCTTACTGT | |
| | TAAAAGACAGGATATGTTTGAAGTGGCTTCTGA | |
| | GAAAAATGGTTAAGAAAATTATGACTTAAAAATG | |
| | TGAGAGATTTTCAAGTATATTAATTTTTTTAACTG | |
| | TCCAAGTATTTGAAATTCTTATCATTTGATTAACA | |
| | CCCATGAGTGATATGTGTCTGGAATTGAGGCCA | |
| | AAGCAAGCTCAGCTAAGAAATACTAGCACAGTG | |
| | CTGTCGGCCCCGATGCGGGACTGCGTTTTGACC | |
| | ATCATAAATCAAGTTTATTTTTTTAATTAATTGAG | |
| | CGAAGCTGGAAGCAGATGATGAATTAGAGTCA | |
| | AGATGGCTGCATGGGGGTCTCCGGCACCCACA | |
| | GCAGGTGGCAGGAAGCAGGTCACCGCGAGAG | |

1. A method for editing the genome of an isolated B lymphocyte comprising the following steps:
(i) activating endogenous activation-induced cytidine deaminase of the B lymphocyte; and
(ii) introducing a DNA molecule comprising a nucleotide sequence encoding a (poly)peptide of interest into the B lymphocyte.

2. The method according to claim 1, wherein the method does not involve an exogenous nuclease and/or an engineered nuclease, such as a CRISPR nuclease, a zinc finger nuclease, a transcription activator-like nuclease or a meganuclease.

3. The method according to claim 1 or 2, wherein the DNA molecule is a linear or linearized DNA molecule.

4. The method according to any one of claims 1-3, wherein the DNA molecule is a single strand DNA molecule (ssDNA) or a double strand DNA molecule (dsDNA).

5. The method according to claim 4, wherein the DNA molecule is dsDNA molecule.

6. The method according to claim 5, wherein the DNA molecule has blunt ends or overhangs.

7. The method according to any one of claims 1-6, wherein the nucleotide sequence of the DNA molecule encoding the (poly)peptide of interest is codon-optimized.

8. The method according to any one of claims 1-7, wherein the DNA molecule comprises an intronic sequence upstream and/or downstream of the nucleotide sequence encoding the (poly)peptide of interest.

9. The method according to claim 8, wherein the intronic sequence comprises a splice recognition site.

10. The method according to claim 8 or 9, wherein the intronic sequence contains Ig-locus intronic sequences.

11. The method according to claim 10, wherein the intronic sequence comprises an intronic sequence of a J-segment downstream intron and/or an intronic sequence of a CH-upstream intron.

12. The method according to any one of claims 1-11, wherein the DNA molecule comprises a splicing enhancer.

13. The method according to any one of claims 1-12, wherein the genome of the B lymphocyte is edited to express a modified immunoglobulin chain comprising in N- to C-terminal direction: a variable domain, the (poly)peptide of interest and a constant domain.

14. The method according to any one of claims 1-13, wherein the genome of the B lymphocyte is edited to express a modified immunoglobulin chain, wherein an endogenous variable domain is replaced by the (poly)peptide of interest.

15. The method according to any one of claims 1-14, wherein the DNA molecule comprises a nucleotide sequence encoding a cleavage site upstream and/or downstream of the nucleotide sequence encoding the (poly)peptide of interest.

16. The method according to claim 15, wherein the cleavage site is a T2A cleavage site.

17. The method according to any one of claims 1-16, wherein the (poly)peptide of interest comprises or consists of a pathogen binding domain, a V_{L} domain, or a V_{H}-V_{L} domain.

18. The method according to any one of claims 1-17, wherein the (poly)peptide of interest comprises or consists of CD4, dipeptidyl peptidase 4, CD9, or angiotensin-converting enzyme 2 or a fragment or sequence variant thereof.

19. The method according to any one of claims 1-18, wherein the isolated B lymphocyte is a primary B lymphocyte.

20. The method according to any one of claims 1-19, wherein the method comprises obtaining an engineered B lymphocyte, wherein the genome of the B lymphocyte comprises the nucleotide sequence encoding the (poly)peptide of interest.

21. The method according to any one of claims 1-20, wherein the method further comprises a step (iii) of confirming integration of the nucleotide sequence encoding the (poly)peptide of interest into the genome of the B lymphocyte.

22. The method according to any one of claims 1-21, wherein the isolated B lymphocyte is cultured in RPMI or IMDM with 10% MS, 1% NEAA, 1% sodium pyruvate, 1% beta-mercaptoethanol, 1% Glutamax, 1% penicillin/streptomycin, 1% kanamycin, and 1% transferrin.

23. The method according to any one of claims 1-22, wherein the isolated B lymphocyte is cultured at 1×10⁵ to 1×10⁶ cells/ml, preferably at 2×10⁵ cells/ml.

24. The method according to any one of claims 1-23, wherein the B lymphocyte is cultured in a culture medium comprising an activator of activation-induced cytidine deaminase.

25. The method according to claim 24, wherein the activator of activation-induced cytidine deaminase is selected from the group consisting of: a cytokine, an anti-B cell receptor antibody or fragments thereof, a TLR agonist, a CpG-B agonist, an imidazoquinoline compound or a combination of any of said activators.

26. The method according to claim 25, wherein the cytokine is selected from the group consisting of CD40L, IL4, IL2, IL21, BAFF, APRIL, CD30L, TGF-β1, 4-1BBL, IL6, IL7, IL10, IL13, c-Kit, FLT-3, IFNα or any combination thereof.

27. The method according to any one of claims 24-26, wherein the cytokine is administered at a concentrations of 0.01-20 ng/ml.

28. The method according to any one of claims 1-27, wherein the B lymphocyte is cultured in a medium comprising IL4 and/or CD40L.

29. The method according to claim 28, wherein activating of the activation-induced cytidine deaminase is performed by co-culture with a CD40L expressing cell line and addition of IL-4.

30. The method according to claim 29, wherein the concentration of IL-4 (in the final culture medium) is 0.005-0.03 ng/ml, preferably 0.01-0.025 ng/ml, more preferably 0.015-0.02 ng/ml and most preferably 0.16 ng/ml.

31. The method according to claim 29 or 30, wherein the CD40L expressing cell line is K562L.

32. The method according to any one of claims 1-31, wherein introducing the DNA molecule into the B lymphocyte is performed up to 10 days after activating the activation-induced cytidine deaminase, preferably up to 7 days after activating the activation-induced cytidine deaminase, more preferably up to 5 days after activating the activation-induced cytidine deaminase, even more preferably up to 2 days after activating the activation-induced cytidine deaminase and most preferably about 1 day after activating the activation-induced cytidine deaminase.

33. The method according to any one of claims 1-32, wherein the method does not comprise transducing the B lymphocyte with a retrovirus.

34. The method according to any one of claims 1-33, wherein the DNA molecule is introduced by nucleofection.

35. The method according to any one of claims 1-34, wherein the B lymphocyte is reactivated after introducing the DNA molecule into the B lymphocyte.

36. The method according to any one of claims 1-35, wherein B cell stimulating agents, for example as defined in claims 25-31, are applied to the B lymphocyte after introducing the DNA molecule into the B lymphocyte.

37. The method according to any one of claims 1-36, wherein the B lymphocyte is treated with a DNA inhibitor capable of blocking alternative-end joining before introducing the DNA molecule into the B lymphocyte.

38. The method according to any one of claims 1-37, wherein the DNA molecule comprising a nucleotide sequence encoding the (poly)peptide of interest is incubated with a Ku protein, such as Ku70/Ku80, before introducing the DNA molecule into the B lymphocyte.

39. The method according to any one of claims 1-38, wherein the DNA molecule comprises a nuclear localization signal, such as SV40 nuclear localization signal.

40. The method according to any one of claims 1-39, wherein the DNA molecule does not comprise a nucleotide sequence encoding GFP or RFP.

41. The method according to any one of claims 1-40, wherein the DNA molecule comprises a promoter.

42. The method according to any one of claims 1-41, wherein the DNA molecule comprises a transcription unit.

43. The method according to any one of claims 1-42, wherein about 24 hours after activation of the activation-induced cytidine deaminase of the B lymphocyte, the B lymphocyte is treated with a nuclease inhibitor, such as Mirin.

44. The method according to any one of claims 1-43, wherein the B lymphocyte is a human B lymphocyte.

45. An engineered B lymphocyte obtainable by the method according to any one of claims 1-44.

46. An engineered B lymphocyte comprising an edited immunoglobulin gene locus comprising a heterologous insert comprising a nucleotide sequence encoding a (poly)peptide of interest inserted in its switch region.

47. The B lymphocytes according to claim 45 or 46, wherein the B lymphocyte is human.

48. The B lymphocyte according to any one of claims 45-47, wherein the switch region of an immunoglobulin gene locus of the B lymphocyte comprises a cleavage site, in particular a T2A cleavage site.

49. The B lymphocyte according to any one of claims 45-48, wherein the switch region of an immunoglobulin gene locus of the B lymphocyte comprises a nucleotide sequence encoding a pathogen binding domain, a V_{H} domain, or a V_{L} domain.

50. The B lymphocyte according to any one of claims 45-49, wherein the switch region of an immunoglobulin gene locus of the B lymphocyte comprises a nucleotide sequence encoding CD4, dipeptidyl peptidase 4, CD9, or angiotensin-converting enzyme 2 or a fragment or sequence variant thereof.

51. The B lymphocyte according to any one of claims 45-50, wherein the B lymphocyte does not express a fluorescent reporter protein.

52. The B lymphocyte according to any one of claims 45-51 for use in medicine.

53. The B lymphocyte for use according to claim 52, wherein the B lymphocyte is engineered according to any one of claims 1-44.

54. The B lymphocyte for use according to claim 52 or 53, wherein the engineered B lymphocyte is administered to a patient.

55. The B lymphocyte for use according to claim 54, wherein the patient receiving the engineered B lymphocyte is the same patient from whom the B lymphocyte was isolated prior to engineering.

56. Method for B cell therapy comprising the following steps:
(a) isolating a B lymphocyte from a patient;
(b) engineering the B lymphocyte according to any one of claims 1-44; and
(c) administering the engineered B lymphocyte to the patient.

57. A cell line of B lymphocytes according to any one of claims 45-51.

58. A method for generating an antibody or a fragment thereof comprising a (heterologous) (poly)peptide of interest, the method comprising the following steps:
(1) providing an engineered B lymphocyte or a B cell line according to any one of claims 45-51 and 57, wherein the B lymphocyte comprises an edited immunoglobulin gene locus comprising a heterologous insert comprising a nucleotide sequence encoding the (poly)peptide of interest inserted in its switch region;
(2) culturing the engineered B lymphocyte or the B cell line; and
(3) isolating the antibody or the fragment thereof comprising the (heterologous) (poly)peptide of interest from the B cell culture.

59. The method according to claim 58, wherein the engineered B lymphocyte is obtained by a method according to any one of claims 1-44.

60. The method according to claim 58 or 59 further comprising characterization of the antibody or antibody fragment, wherein characterization comprises
Performing functional assays to determine the function of the antibody or antibody fragment;
Performing binding assays to determine the binding specificity of the antibody or antibody fragment and/or the binding partner/epitope recognized by the antibody or antibody fragment; and/or
Performing neutralization assays to determine the ability of the antibody or antibody fragment to neutralize a toxin or a pathogen.

61. Antibody obtainable by the method according to any one of claims 58-60.

62. A composition comprising the B lymphocyte according to any one of claims 45-51 or the antibody according to claim 61.

63. The composition according to claim 62 further comprising a pharmaceutically acceptable carrier.

64. The composition according to claim 62 or 63 for use in medicine.

65. A method for immunotherapy comprising administration of the antibody according to claim 61, the engineered B cell according to any one of claims 45-51 or the composition according to claim 62 or 63 to a subject in need thereof.
